(19) United States
(12) Patent Application Publication
(10) Pub. No.: US 2024/0336952 A1
(43) Pub. Date: Oct. 10, 2024

(54) EXTRACHROMOSOMAL CIRCULAR DNA AS AN IMMUNOSTIMULANT AND BIOMARKER FOR DISEASE

(71) Applicant: CHILDERN'S MEDICAL CENTER CORPORATION, Boston, MA (US)

(72) Inventors: Yi Zhang, Newton, MA (US); Yuangao Wang, Brookline, MA (US)

(73) Assignee: CHILDERN'S MEDICAL CENTER CORPORATION, Boston, MA (US)

(21) Appl. No.: 18/579,325

(22) PCT Filed: Jul. 15, 2022
(86) PCT No.: PCT/US22/73785

§ 371 Date: Jan. 12, 2024

Related U.S. Application Data
(60) Provisional application No. 63/222,386, filed on Jul. 15, 2021.
(60) Provisional application No. 63/317,879, filed on Mar. 8, 2022.
(60) Provisional application No. 63/222,386, filed on Jul. 15, 2021.
(60) Provisional application No. 63/317,879, filed on Mar. 8, 2022.

(51) Int. Cl. C12Q 1/6806; C12N 15/10; C12Q 1/44; C12Q 1/6844

(52) U.S. Cl. CPC C12Q 1/6806; C12N 15/1013; C12Q 1/44; C12Q 1/6844; G01N 2333/922

(57) ABSTRACT

Provided herein are compositions comprising circular DNA derived from chromosomal DNA of a eukaryote (eccDNA), as well as methods for producing and administering such compositions for the purpose of simulating an immune response in an individual.

### RELATED APPLICATIONS

This Application claims the benefit under 35 U.S.C. § 119(e) of U.S. Provisional Application Ser. No. 63/222,386 entitled “EXTRACHROMOSOMAL CIRCULAR DNA AS AN IMMUNOSTIMULANT AND BIOMARKER FOR DISEASE,” filed on Jul. 21, 2021, and U.S. Provisional Application Ser. No. 63/317,879 entitled “EXTRACHROMOSOMAL CIRCULAR DNA AS AN IMMUNOSTIMULANT AND BIOMARKER FOR DISEASE,” filed on Mar. 8, 2022, the entire contents of which are incorporated herein by reference.

REFERENCE TO AN ELECTRONIC SEQUENCE LISTING The contents of the electronic sequence listing (C123370209WO00-SEQ-RE.xml; Size: 52,779 bytes; and Date of Creation: Jul. 15, 2022) is herein incorporated by reference in its entirety.

### BACKGROUND OF INVENTION

Extrachromosomal circular DNA (eccDNA) is a double stranded DNA species that is produced by a variety of eukaryotes. Suggested functions for eccDNA range from involvement in the repair of DNA damage, hyper-transcription, homologous recombination, stress during replication, gene expression during cancer, and aging.

### SUMMARY OF INVENTION

Programmed cell death, or apoptosis, is vital for the development and maintenance of many multicellular species, including humans. Apoptosis also produces a myriad of molecular byproducts which are of potential use for a range of applications. For example, as described, some apoptotic products are immunostimulatory when administered to an individual or are indicators of cell death occurring in an individual. Of particular interest is extrachromosomal circular DNA (eccDNA), which is useful as both a biomarker of diseases associated with apoptosis, cell death, as well as an immunostimulant that may be administered to individuals to treat or protect against disease.

Provided herein are methods of producing extrachromosomal circular DNA (eccDNA) and compositions which comprise eccDNA, a double stranded nucleic acid species produced by many multicellular eukaryotes, particularly in response to apoptosis. Extrachromosomal circular DNA (eccDNA) is immunostimulatory. Methods for the isolation of eccDNA of high purity are provided. In addition, methods for producing circular DNA species that are functionally equivalent to eccDNA and compositions thereof are also provided. eccDNA, such as that obtained by the methods described, may be used in a variety of applications. In some embodiments, levels of eccDNA may be assessed as a biomarker for various diseases (e.g., sepsis) associated with cell death in an individual. In some embodiments, the presence of eccDNA may be assessed as a biomarker for various diseases (e.g., sepsis) associated with cell death in an individual. In other embodiments, compositions comprising eccDNA or circular DNA having essentially the same properties as eccDNA (referred to as an eccDNA equivalent), such as a synthetically produced eccDNA equivalent, are used to stimulate an immune response in an individual. In some embodiments, compositions which comprise eccDNA or an eccDNA equivalent can be administered to an individual to elicit, enhance, prolong, or modulate immune responses to antigens in the individual in order to maximize protective immunity.

One aspect of the present invention pertains to a method for enriching eccDNA from a mixture, said method comprising obtaining a mixture comprising DNA (e.g., circular and linear DNA); treating the mixture with an enzyme that linearizes circular DNA that is not eccDNA, under conditions suitable for linearizing such DNA (under conditions under which such DNA is linearized); treating the resulting mixture with an enzyme that digests linear DNA to produce a mixture of digested linear DNA and eccDNA, and separating eccDNA from the mixture of digested linear DNA and eccDNA, thereby producing enriched eccDNA.

In another aspect, the present invention pertains to a method for enriching eccDNA from a sample, said method comprising obtaining a sample comprising eccDNA; treating the sample with an enzyme that linearizes mitochondrial DNA (mtDNA), under conditions suitable for linearizing mtDNA (under conditions under which such DNA is linearized); treating the resulting mixture with an enzyme that digests linear DNA, which results in production of a mixture comprising digested linear DNA and eccDNA; and separating eccDNA from the mixture of digested linear DNA and eccDNA, thereby producing enriched eccDNA.

In some embodiments, the sample is a biological sample collected from an individual. In some embodiments, the biological sample is a blood or plasma sample. In some embodiments, the individual is a mammal. In some embodiments, the individual is a human.

In some embodiments the sample comprises a population of cells (e.g., mammalian cells, such as human cells). In some embodiments, the sample comprises a population of cultured cells. In some embodiments, the population of cultured cells comprises mammalian cells, such as human cells. In some embodiments, cells in the sample are fixed prior to treating the sample with an enzyme that linearizes mtDNA. In some embodiments, cells in the sample are lysed prior to treating the sample with an enzyme that linearizes mtDNA. In some embodiments, cells in the sample are lysed by buffered alkaline lysis conducted at a pH between 11.0 and 12.3, or, in specific embodiments, conducted at a pH of about 11.8. In some embodiments, buffered alkaline lysis is conducted in the presence of a compound that has an acid dissociation pH (pKa) between 11.0 and 12.3. In some embodiments, the compound is pyrrolidine.

In some embodiments, the unenriched eccDNA is bound to magnetic silica beads to separate eccDNA from lysed cells in the sample prior to treating the sample with an enzyme that linearizes mtDNA.

In some embodiments, the enzyme that linearizes mtDNA is PacI.

In some embodiments, the enzyme that digests linear DNA such as linearized mtDNA is plasmid safe DNase or Exonuclease V.

In some embodiments, the linearization of mtDNA and the digestion of linear DNA are conducted concurrently.

In some embodiments, the eccDNA is separated from the digested linear mtDNA by phenol/chloroform/isoamyl alcohol (PCI) extraction.

In some embodiments, the eccDNA is separated from the digested linear mtDNA by contacting the mixture with magnetic silica beads.

In some embodiments, the method further comprises amplifying enriched eccDNA by rolling circle amplification.

In another aspect, the present invention pertains to a composition comprising eccDNA and a pharmaceutically acceptable excipient. In some embodiments, the composition comprises eccDNA produced by any one of the methods described herein.

In some embodiments, the composition comprises eccDNA that is derived from chromosomal DNA of a mammal. In some embodiments, the composition comprises eccDNA that is derived from chromosomal DNA of a human.

In some embodiments, the composition comprises eccDNA that is essentially free of bacterial, bacteriophage, and plasmid sequences. In some embodiments, the composition comprises eccDNA that is essentially free of site-specific recombination sites. In some embodiments, the composition comprises eccDNA that is non-replicating. In some embodiments, the composition comprises eccDNA that has a size range of about 70 nucleotides to about 2000 nucleotides.

In some embodiments, the composition further comprises an antigen. In some embodiments, the antigen is a peptide, protein, or nucleic acid. In some embodiments, the antigen is an antigen from a pathogen. In some embodiments, the pathogen is a bacterium, a virus, or a parasite.

In another aspect, the present invention pertains to a composition comprising circular DNA and a pharmaceutically acceptable excipient, wherein the circular DNA is non-replicating and does not comprise a gene that is capable of being expressed.

In some embodiments, the composition comprises circular DNA that comprises a random DNA sequence. In some embodiments the composition comprises circular DNA that comprises an entirely random DNA sequence.

In some embodiments, the composition comprises circular DNA that has a size range of about 70 nucleotides to about 2000 nucleotides.

In some embodiments, the composition comprises circular DNA that is synthesized in vitro.

In some embodiments, the composition further comprises an antigen. In some embodiments, the antigen is a peptide, protein, or nucleic acid. In some embodiments, the antigen is an antigen from a pathogen. In some embodiments, the pathogen is a bacterium, a virus, or a parasite.

In another aspect, the present invention pertains to a method for eliciting an immune response in an individual, said method comprising administering to the individual an effective amount of eccDNA, such as in a composition comprising an effective amount of immunostimulatory circular DNA (e.g., an effective amount of eccDNA) and a pharmaceutically acceptable excipient, wherein the immunostimulatory circular DNA is non-replicating and does not comprise a gene that is capable of being expressed.

In some embodiments, the immunostimulatory circular DNA has been obtained from the individual to whom it is administered. In some embodiments, the immunostimulatory circular DNA has been obtained from a second individual (an individual other than the individual to whom it is administered). In some embodiments, the immunostimulatory circular DNA has been obtained from a population of cultured cells. In some embodiments, the immunostimulatory circular DNA comprises eccDNA derived from chromosomal DNA of a mammal. In some embodiments, the immunostimulatory circular DNA comprises eccDNA derived from chromosomal DNA of a human. The eccDNA can be “derived from” chromosomal DNA in that it is obtained from DNA in cells or an ancestor thereof (e.g., using selection, isolation, amplification techniques) or in that the sequence of the chromosomal DNA is used to produce the sequence of the eccDNA (e.g., the eccDNA sequence is the same/essentially the same as the sequence of the chromosomal DNA).

In some embodiments, the immunostimulatory circular DNA has been synthesized in vitro. In some embodiments, the immunostimulatory circular DNA comprises a random sequence. In some embodiments, the immunostimulatory circular DNA comprises an entirely random sequence.

In some embodiments, the immunostimulatory circular DNA has been amplified by rolling circle amplification.

In some embodiments, the immunostimulatory circular DNA is essentially free of bacterial, bacteriophage, and plasmid sequences. In some embodiments, the immunostimulatory circular DNA is essentially free of sequences encoding site-specific recombination sites. In some embodiments, the immunostimulatory circular DNA has a size range of about 70 nucleotides to about 2000 nucleotides.

In some embodiments, the composition comprising immunostimulatory circular DNA administered to the individual further comprises an antigen. In some embodiments, the antigen is a peptide, protein, or nucleic acid. In some embodiments, the antigen is an antigen from a pathogen. In some embodiments, the pathogen is a bacterium, a virus, or a parasite.

In some embodiments, administration of the composition to the individual activates cGAS-STING signaling in cells of the individual. In some embodiments, administration of the composition to the individual elicits an innate immune response in the individual. In some embodiments, administration of the composition to the individual elicits a cell-mediated immune response in the individual. In some embodiments, administration of the composition to the individual elicits proliferation of mature T helper type 1 (Th1) cells. In some embodiments, administration of the composition to the individual elicits an increase in inflammatory cytokines. In some embodiments, administration of the composition to the individual elicits an increase in one or more inflammatory cytokines selected from interferon alpha (IFNα), interferon beta (IFNβ), interferon gamma (IFNγ), interleukin 6 (IL-6), and tumor necrosis factor alpha (TNFα).

In some embodiments, the immune response is elicited in the individual to treat a disease or condition. In some embodiments, the immune response is elicited in the individual to prevent a disease or condition or reduce the extent to which the disease or condition occurs. In some embodiments, the disease or condition is cancer. In some embodiments, the disease or condition is caused by a pathogen. In some embodiments, the disease or condition is caused by a bacterium, a virus, or a parasite.

In some embodiments, the administration is intravenous, intramuscular, intradermal, intranasal, topical, or oral.

In some embodiments, the individual is a mammal. In some embodiments, the individual is a human.

In another aspect, the present invention pertains to an immunostimulatory composition comprising immunostimulatory circular DNA and a pharmaceutically acceptable excipient, wherein the immunostimulatory composition elicits an immune response in an individual when administered to the individual, and wherein the circular DNA is non-replicating and does not comprise a gene that is capable of being expressed.

In some embodiments, the immunostimulatory composition comprises immunostimulatory circular DNA that comprises eccDNA derived from chromosomal DNA of a mammal. In some embodiments, the immunostimulatory composition comprises immunostimulatory circular DNA that comprises eccDNA derived from chromosomal DNA of a human.

In some embodiments, the immunostimulatory composition comprises immunostimulatory circular DNA that is synthesized in vitro. In some embodiments, the immunostimulatory composition comprises immunostimulatory circular DNA that comprises a random sequence. In some embodiments, the immunostimulatory composition comprises immunostimulatory circular DNA that comprises an entirely random sequence.

In some embodiments, the immunostimulatory composition comprises immunostimulatory circular DNA that is essentially free of bacterial, bacteriophage, and plasmid sequences. In some embodiments, the immunostimulatory composition comprises circular DNA that is essentially free of sequences encoding site-specific recombination sites. In some embodiments, the immunostimulatory composition comprises immunostimulatory circular DNA that has a size range of about 70 nucleotides (base pairs) to about 2000 nucleotides (base pairs).

In some embodiments, the immunostimulatory composition further comprises an antigen. In some embodiments, the antigen is a peptide, protein, or nucleic acid. In some embodiments, the antigen is an antigen from a pathogen. In some embodiments, the pathogen is a bacterium, a virus, or a parasite. In some embodiments, the circular DNA enhances the immunogenicity of the antigen when co-administered to an individual.

In some embodiments, administration of the immunostimulatory composition to the individual activates cGAS-STING signaling in cells of the individual. In some embodiments, administration of the immunostimulatory composition to the individual elicits an innate immune response in the individual. In some embodiments, administration of the immunostimulatory composition to the individual elicits a cell-mediated immune response in the individual. In some embodiments, administration of the immunostimulatory composition to the individual elicits proliferation of mature T helper type 1 (Th1) cells in the individual. In some embodiments, administration of the immunostimulatory composition to the individual elicits an increase in inflammatory cytokines in the individual.

In some embodiments, the individual is a mammal. In some embodiments, the individual is a human.

In another aspect, the present invention pertains to a method for assessing a disease in an individual known, suspected to have, or at risk for a disease associated with an increase in the level of eccDNA, said method comprising obtaining a sample comprising eccDNA from the individual, measuring the level of eccDNA in the sample, and comparing the level of eccDNA measured in the sample to a reference level of eccDNA for the disease, wherein a statistical similarity between the level of eccDNA measured in the sample and the reference level is indicative of the disease.

In some embodiments, the sample is a blood or plasma sample.

In some embodiments, the eccDNA is enriched by one of the methods described herein.

In some embodiments, the individual is human.

In some embodiments, the disease is caused by a pathogen. In some embodiments, the disease is sepsis, acute respiratory distress syndrome (ARDS), CAR T cell-induced cytokine release syndrome (CRS), or coronavirus disease 2019 (COVID-19).

### BRIEF DESCRIPTION OF DRAWINGS

The accompanying drawings are not intended to be drawn to scale. In the drawings, each identical or nearly identical component that is illustrated in various FIGs. is represented by a like numeral. For purposes of clarity, not every component may be labeled in every drawing.

In the drawings:

FIGS. 1A to 1E: A three-step eccDNA purification procedure. FIG. 1A: Schematic of the three-step eccDNA purification and sequencing procedure. Step 1, crude DNA circles were extracted in a buffered alkaline lysis and bound to silica column; Step 2, mtDNA was linearized by PacI and all linear DNA was removed by digestion with Plasmid Safe DNase (P.S. DNase); Step 3, eccDNAs were further separated from residual linear DNA by selective binding to magnetic silica beads in Solution A; resulting eccDNAs were sequenced by Oxford Nanopore (left) after RCA or directly tagmented with Tn5 for Illumina sequencing without RCA (right). FIG. 1B: Agarose gel image showing eccDNAs purified from over-confluent HeLa without (−) or with (+) PacI treatment. 1% vertical agarose gel was stained by SYBR Gold. M, linear DNA maker. FIG. 1C: EccDNAs in FIG. 1B were spread on freshly cleaved mica and scanned with scanning atomic force microscope (SAFM). Shown are two representative fields. Black bar=0.2 μm. FIG. 1D: Agarose gel image showing eccDNAs purified from normal cultured mESCs were fractionated as in FIG. 1B. Arrowheads indicate distinct DNA bands. FIG. 1E: EccDNAs in FIG. 1D were spread on freshly cleaved mica and scanned with SAFM. Shown are two representative fields. Black bar=0.2 mm.

FIGS. 2A to 2B: Selective binding of eccDNAs to beads and high sensitivity of DNA detection by SYBR Gold staining. FIG. 2A: Representative gel image showing selective binding of circular DNA to magnetic silica beads in solution A. The assay was tested with 200× (mass) linear DNA (lane 4 and 6) and 1× (mass) DNA circles (lane 5 and 6). Lanes 1-3 indicate the input DNAs. Lane 1, 0.5% of input linear DNA for lane 4 was loaded; lane 2, equal amount of input circular DNAs for lane 5 were loaded; lane 3, 0.5% of input linear DNA and equal amount of input circular DNAs for lane 6 were loaded. Lanes 4-6 were DNAs recovered from Solution A. lane 4, 200× (mass) linear DNA alone undergone purification by solution A; lane 5, 1× (mass) DNA circles undergone purification by solution A; lane 6 mixture of 200× (mass) linear DNA and 1× (mass) DNA circles undergone purification by solution A. Note, only circular DNAs were recovered. The recovery rate is very high, particularly for the smaller circular DNAs. FIG. 2B: High sensitivity of DNA detection using vertical agarose gel electrophoresis and SYBR Gold staining. 0.2 μL commercial DNA ladder (total of 10 ng) was undergone 5× series dilutions and fractionated by 1% vertical agarose gel electrophoresis, stained by SYBR Gold, DNA in lane-4 were 125× diluted of that in lane-1, and could still be visualized. The detection limit is estimated as 5-10 pg/band.

FIGS. 3A-3F: EccDNAs are circularized genomic DNA fragments that mapped across the genome. FIG. 3A: Examples of eccDNAs shown in Integrative Genomics Viewer (IGV). EccDNAs in two genomic loci of chromosomes 18 and 17 were shown. Gray areas under the chromosomal coordinates show overall coverage of nanopore long reads. Each horizontal bar represents a sub-read of a nanopore long read that was repeatedly aligned to the same genomic locus indicated by the same shade. The 4 circles represent eccDNAs retrieved from Nanopore reads. ecc-1, ecc-2 and ecc-4 are all single fragment circles; ecc-1 and ecc-2 are overlapped; ecc-3 is a two-fragment circle (2f ecc) with fragments from chromosome 18 and 17, respectively; ecc-4 overlaps with one fragment of ecc-3. FIG. 3B: Histogram showing the eccDNA size distribution and relative abundance. FIG. 3C: Pie chart showing the percentages of eccDNA with the indicated event number in the total unique eccDNAs identified. FIG. 3D: Bar plot showing eccDNA counts with the number of fragments (1-7) in each circle. FIG. 3E: Circle plot showing chromosomal originations of all 2 fragments eccDNAs (2f ecc). Genomic fragments from the same chromosome are indicated with the same shade. FIG. 3F: Overall chromosomal distribution of eccDNAs across the mouse genome.

FIGS. 4A-4D: Summary of nanopore sequencing data. FIG. 4A: Diagram of nanopore long read sequencing of eccDNA. Tandem copies of eccDNAs were self-concatenated to long molecule by rolling cycle amplification (RCA), and directly read through by Oxford Nanopore. Each copy of eccDNA molecule in a single long molecule was sequenced multiple times. “S”: split site. FIG. 4B: Summary of mESC eccDNA long reads from an Oxford Nanopore MinION flow cell. FIG. 4C: Diagram showing how the eccDNA full-length sequence is called and categorized. Nanopore reads from FIG. 4A, the dashed box, were aligned to a single locus (Continuous) or to multiple loci (Non-continuous) in genome. Continuous: an example of six sub-regions of a long nanopore read repeatedly aligned to a single locus, where full length sequence of eccDNA was presented with only one split site (S); Non-continuous: an example of eight sub-regions of a single long read sequentially aligned to two separate loci (Locus-1 and Locus-2), representing an eccDNA ligated by two genomic fragments (2f ecc) with two split sites (S1 and S2). FIG. 4D: Criteria for eccDNA calling. EccDNAs were called based on their number of full passes aligned to the genome. Long reads with less than two full passes were discarded (left and middle panel). Left, Nanopore read that hits genome only once either fails to designate the genomic start and end site of eccDNA (up panel), or miss the middle region (bottom panel) that may or may not include in the original eccDNA molecule (Uncertain); Middle, because of potential sequencing error of Oxford Nanopore, reads that hit genome more than once but less than twice (1 full pass) were also discarded due to the lack of confirmation in eccDNA calling, particularly on designating the start and end site of eccDNA. Right, eccDNA molecules were called from long reads when covered at least twice (>=2 full pass) on their aligned loci.

FIGS. 5A-5B: Summary of Illumina short read sequencing and Chromosomal distribution of eccDNA fragments. FIG. 5A: Summary of eccDNA short read sequencing without RCA. Purified eccDNA was directly tagmented with Tn5 (Illumina Nextera), and sequenced with Illumina Hiseq 2500 in PE150 mode. FIG. 5B: Chromosomal distribution of eccDNA short reads.

FIGS. 6A-6C: Apoptotic oligonucleosomal DNA fragmentation is required for eccDNA production in mESCs. FIG. 6A: EccDNA production is induced by apoptosis. Oligonucleosomal DNA fragmentation (left) and eccDNAs (right) were induced by apoptosis inducers. Equal number of mESCs were treated with the indicated apoptosis inducers, total DNA (a) and eccDNAs (b) were purified and visualized with SYBR Gold staining after vertical agarose gel electrophoresis. STS: Staurosporine; ETO: etoposide; UV: ultraviolet. Equal volume was loaded in each lane, and the loading volume was determined using the UV treated sample to avoid overloading. FIG. 6B: Deficiency of DNase γ or EndoG in mESC do not significantly alter UV-induced cell death. Cell death was measured by flow cytometry after staining with Live/Dead Cell Stain Kit. Error bars indicate standard deviation from 3 independent experiments. Data is presented as mean±S.D. of three independent experiments. ANOVA with post-hoc Bonferroni correction. ns, not significant. FIG. 6C: Deficiency of oligonucleosomal DNA fragmentation abolishes apoptosis induced eccDNA production. Deficiency of DNase γ (DNase gamma), but not EndoG in mESCs, abolishes UV-induced oligonucleosomal DNA fragmentation (left), and eccDNA production (right). EccDNA were purified from equal amount of UV irradiated cells, mitochondrial DNA (Mt) was kept as an inner control.

FIGS. 7A-7E: Generation of Endonuclease G and DNase γ knockout cell line by CRISPR/Cas9. FIG. 7A: Quantification of eccDNA from cells of the indicated treatment. Presented are the total nanogram per 10 million cells. Bars indicate mean±S.D. of three independent experiments. FIG. 7B: Diagram illustration and PCR confirmation of the EndoG knockout mESC line. Gene structure of EndoG, sgRNAs (gray arrows), two sets of screen primers for internal sites (black arrowhead, “n”=negative knockout cell line) and external sites (gray arrowhead, “p”=positive knockout cell line) were shown. FIG. 7C: Diagram illustration and PCR confirmation of the DNase γ knockout mESC lines. Gene structure of DNase γ, sgRNAs (gray arrows), two sets of screen primers for internal sites (black arrowhead, “n” =negative knockout cell line) and external sites (gray arrowhead, “p”=positive knockout cell line) were shown. FIG. 7D: Cell viability is not affected by EndoG or DNase γ KO. Cell viability was evaluated by flow cytometry after staining with Live/Dead Cell Stain Kit. Error bar indicates S.D. of three independent experiments. FIG. 7E: Quantification of eccDNAs presented in FIG. 6C. EccDNAs below the mtDNA band were quantified by densitometry and presented as relative levels to that in WT cells. Bars indicate mean±S.D. of three independent experiments.

FIGS. 8A-8C: Lig3 is required for circularization of fragmented DNA for eccDNA formation. FIG. 8A: Confirmation of DNA ligases deficient cell lines. Up, Western blot analysis of individual DNA ligases and their combinational knockout in CH12F3 cell lines. Bottom, genomic structure of DNA ligase 3 with CRISPR/cas9 specific targeting (Δ) the nuclear isoform of Lig3 (NucLig3−/−) without affecting the mitochondrial isoform (MtLig3) for cell viability. FIG. 8B: Agarose gel electrophoresis showing staurosporine induced oligonucleosomal DNA fragmentation in the indicated CH12F3 cell lines (left) and representative gel showing eccDNAs purified from equal amount of CH12F3 cells with the indicated genotype (right); Mt: mitochondrial DNA. FIG. 8C: Quantification of eccDNA below the mtDNA band by densitometry. Data is presented as relative levels to that purified from WT cells (FIG. 8B); bars indicate mean±S.D. of three independent experiments. **P<0.01; ANOVA with post-hoc Bonferroni correction.

FIGS. 9A-9F: EccDNAs induce transcription of cytokine genes in BMDCs. FIG. 9A: Bar graphs showing the fold of induction of cytokine gene expression with varying level of eccDNA, compared with fragmented linear DNA, and poly(dG:dC). Equal amount of the indicated DNA was transfected to BMDC at the indicated concentration. 12 hours after transfection, total RNAs were extracted and analyzed by RT-qPCR. Expression level of the indicated genes is presented as fold change (FC) relative to that in mock transfection (without DNA) after normalization to GAPDH. Linear genomic DNA (Linear) was prepared by sonicating genomic DNA to sizes similar to that of eccDNAs; poly(dG:dC), poly(deoxyguanylic-deoxycytidylic) from InvivoGen. Representative data is shown as mean±SD of three independent experiments. FIG. 9B: ELISA analysis of IFN-α, IFN-β, and IL-6 in FIG. 9A. nd, not detected. Data is shown as mean±SD of replicates of a representative experiment of three independent experiments. FIGS. 9C-9D: EccDNAs induce cytokine genes in BMDMs. Bar graphs showing the induction of mRNA (FIG. 9C) and protein (FIG. 9D) of IL-6 and TNF-α in BMDMs that transfected with varying levels of eccDNA, compared with fragmented linear DNA, and poly(dG:dC). RT-qPCR were performed as in (FIG. 9A) after 12-hours transfection. ELISA analysis (FIG. 9D) was performed after 24-hours transfection. Data is shown as mean±SD of replicates of a representative experiment of three independent experiments. FIGS. 9E-9F: DNase I pretreatment abolishes IL-6 and TNF-α induction by eccDNA. Equal amount of DNA (120 ng/ml) used to prepare transfection mixtures were pre-treated with or without DNase I as indicated, then transfected to BMDCs. 12 hours later, total RNA was extracted for RT-qPCR (FIG. 9E) and medium was collected for ELISA (FIG. 9F). Data is shown as mean±SD of replicates of a representative experiment in three independent experiments. *P<0.05; ns, no significant; ANOVA with Bonferroni correction.

FIGS. 10A-10E: EccDNAs are potent immunostimulants. FIG. 10A: Various DNAs were resolved by agarose gel electrophoresis. Linear: sheared linear genomic DNA; poly(dG:dC), poly(deoxyguanylic-deoxycytidylic). FIG. 10B: Confirmation of BMDC identity by flow cytometry. After differentiating bone marrow cells with 20 ng/ml GM-CSF for 7 days, cells were first gated based on their sizes (left), and then phenotyped on the basis of their CD11c and major histocompatibility complex II (MHCII) expression (right) to define BMDC, the numbers indicate the percentage of gated cells. Data shown are representative of 2 independent experiments. FIGS. 10C-10D: Bar graphs showing the relative mRNA (FIG. 10C) and protein (ELISA) (FIG. 10D) levels of IFN-α. Data is shown as mean±SD of replicates of a representative experiment in three independent experiments. nd, not detected. *P<0.05; ANOVA with Bonferroni correction. FIG. 10E: Confirmation of BMDMs identify by flow cytometry. After differentiating bone marrow cells with L929 conditioned medium for 7 days, cells were first gated based on their sizes (left), and then phenotyped on the basis of F4/80 and CD11b expression (right) to define BMDM, the numbers indicate the percentage of gated cells. Data shown is representative of 2 independent experiments.

FIGS. 11A-11F: The circularity of eccDNAs, but not the sequence, is critical for their immunostimulant activity. FIG. 11A: Representative gel confirming eccDNA linearization. Equal amount of the indicated DNA was digested with or without plasmid safe DNase (P.S.), and then visualized by SYBR Gold staining after agarose gel electrophoresis. Linear: sonicated linear genomic DNA; Li-ecc: linearized eccDNA. Experiments were performed for at least three times. FIG. 11B: Linearized eccDNAs lost their immunostimulatory activities. DNA of lanes 1-3 from FIG. 11A were transfected at 30 ng/ml to BMDCs. 12 hours later, RNAs were isolated for RT-qPCR. The fold changes of expression are presented as above. A representative experiment shown as mean±SD of replicates of three independent experiments. *P<0.05; ANOVA with Bonferroni correction. FIG. 11C-11D: Synthetic small DNA circles are potent immunostimulants. Equal amount of the indicated synthetic linear (Syn-linear) and circular (Syn-circular) DNAs were transfected to BMDCs at the indicated concentration. 12 hours post-transfection, total RNA was extracted and used for RT-qPCR to evaluate the fold changes of mRNA level (FIG. 11C), and the medium was collected for ELISA (FIG. 11D). Synthetic linear and circular DNA had the same randomly designated sequence; poly(dG:dC), poly(deoxyguanylic-deoxycytidylic). Representative data is shown as mean±SD of replicates in three independent experiments. *P<0.05; ns, no significant; ANOVA with Bonferroni correction. FIG. 11E: EccDNAs are present in supernatant of apoptotic medium of wild-type (WT), but not in that of DNase γ −/− (γ−/−) cells. Supernatant of apoptotic medium was filtrated with 0.45 um filter and used for eccDNA purification. Left, representative gel of eccDNA in the supernatant of apoptotic medium. Right, quantification of eccDNA in the supernatant of the indicated cells of three independent experiments. nd, not detected. Data is shown as mean±SD of three independent experiments. *P<0.05; T-test. FIG. 11F: Exonuclease resistant DNA (not mtDNA) in the supernatant of apoptotic medium can activate BMDCs. Apoptotic medium from both WT and DNase γ (DNase gamma) −/− cells were used to treat BMDCs. 12 hours later, total RNA was purified and RT-qPCR was performed. P.S.: plasmid safe DNase, PacI: restriction enzyme that can linearize mitochondrial DNA, Benz: Benzonase, nuclease that can destroy all forms of DNA and RNA. Data is shown as mean±SD of replicates of a representative in three independent experiments. *P<0.05; ANOVA with Bonferroni correction.

FIGS. 12A-12F: The circularity of eccDNAs, but not the sequence, is critical for their immunostimulant activity. FIG. 12A: Bar graphs showing the relative TNF-α mRNA level. Data is shown as mean±SD of replicates of a representative experiment in three independent experiments. *P<0.05; ANOVA with Bonferroni correction. FIGS. 12B-12C: Bar graphs showing the relative TNF-α mRNA level (FIG. 12B), and protein (ELISA) level (FIG. 12C). Data is shown as mean±SD of replicates of a representative experiment in three independent experiments. *P<0.05; ANOVA with Bonferroni correction. FIG. 12D: Diagram of DNA transfection efficiency and stability assay. Step 1: 5 Phosphorothioate (“*”) end-protected synthetic linear DNA (PS-syn-linear) and its circular form with equal number of phosphorothioate bonds (PS-Syn-circular) were transfected to BMDCs in the same way as in FIG. 11 for either 1 or 12 hours. Step 2: cells were lysed in 100 μl lysis buffer and treated with Thermoliable Proteinase K to prepare total DNA. Step 3: 4 μl total DNA was used for qPCR with a set of primers that amplify a 117 bp fragment in both linear and circular DNA. FIG. 12E: qPCR analysis of the samples prepared as described in (FIG. 12D). Transfected DNA level was normalized to that of 10 ng/ml PS-syn-linear transfection. Bars indicate S.D. of three independent experiments. ns, not significant; ANOVA with Bonferroni correction. FIG. 12F: 12 hours after transfection of DNA at 30 ng/ml, medium was collected for ELISA essay, Data is shown as mean±SD of replicates of a representative experiment in three independent experiments. *P<0.05; ANOVA with Bonferroni correction.

FIGS. 13A-13F: Sting is required for eccDNA induced gene expression. FIG. 13A: Scatter plot showing 290 genes (left panel, dots above upper dashed line) that are significantly induced (fold change ≥5 and adjusted p-value <0.001) by eccDNA, but not linear DNA, in BMDC. 34 significantly induced cytokine genes are indicated (right panel, black dots). The x and y-axis are log 2-transformed normalized read counts. FIG. 13B: Heatmap presentation of the top 20 induced genes. FIG. 13C: GO terms enriched in the genes activated by eccDNA treatment in BMDC. The number of genes in each term and the p-values of the enrichment are indicated. FIG. 13D: Scatter plot indicates that the transcriptomes of BMDC treated with eccDNAs and synthetic circular DNA are highly similar (FC ≥2, adjusted p-value <0.01). FIG. 13E: Heatmap presentation of the eccDNA response genes in control and eccDNA treated BMDC of the indicated genotypes. FIG. 13F: Scatter plots comparing the transcriptome affected by eccDNA in BMDC of WT, Sting−/−, and Myd88−/− mice. EccDNA response genes in WT BMDC are indicated by dots above upper dashed line (up-regulated, n=365) and dots below lower dashed line (down-regulated, n=79). The x and y-axis are log 2-transformed normalized read counts. (FC ≥5, adjusted p-value <0.001).

FIGS. 14A-14H: Global transcriptional response to eccDNA in BMDCs and BMDMs. FIG. 14A: Pearson correlation co-efficient of pair-wise comparisons of transcriptomes of 3 replicates of mock treated (control), eccDNA treated, and linear DNA treated BMDCs. FIG. 14B: Pearson correlation co-efficient of pair-wise comparisons of transcriptomes of 3 replicates of mock treated (control), eccDNA treated, and linear DNA treated BMDMs. FIG. 14C: Scatter plot showing 380 genes (left panel, dots above upper dashed line) that are significantly induced (FC ≥5 and adjusted p-value <0.001) by eccDNA, but not linear DNA, in BMDMs. 24 significantly induced cytokine genes are indicated (right panel, black dots). The x and y-axis are log 2-transformed normalized read counts. FIG. 14D: Heatmap presentation of the top 20 induced genes. FIG. 14E: GO terms enriched in the genes activated by eccDNA treatment in BMDMs. The number of genes in each term and the p-value of the enrichment is indicated. FIG. 14F: Scatter plot indicates the transcriptomes of the two replicates of synthetic circular DNA treated BMDCs are highly similar. FIG. 14G: Western blot confirmation of the Sting−/− and Myd88−/− cells. FIG. 14H: Correlation of the transcriptomes of 2 replicates eccDNA treated Sting−/− and Myd88−/− BMDC.

FIGS. 15A to 15B: Scheme of 3SEP eccDNA purification. FIG. 15A: A schematic showing the 3SEP scheme. Step 1: crude DNA circles are extracted from whole cells with a buffered alkaline lysis at pH 11.8 (Buffer 2), bound and eluted from either silica column or magnetic silica beads; Step 2: linear DNA and PacI (optionally) linearized mitochondrial DNA are digested with Plasmid-Safe (P.S.) DNase; Step 3: eccDNAs are selectively recovered with magnetic beads and Solution CS. FIG. 15B: Selective recovery of circular DNA by using Solution CS. Equal amount of linear DNA ladder (NEB) and supercoiled DNA ladder (NEB) were recovered by Solution CS, recovery rates were calculated as percentage of the input.

FIGS. 16A to 16B: Microscopy imaging and quantification of eccDNAs. FIG. 16A: Atomic force microscopy images of the eccDNAs purified from HeLa cells after removing mtDNA by PacI treatment. Bar size=500 nm. FIG. 16B: EccDNAs shown in FIG. 16A were separated in a 1.2% agarose gel and stained with Sybr Gold for visualization.

### DETAILED DESCRIPTION OF CERTAIN EMBODIMENTS

Some aspects of the present disclosure are based, at least in part, on the demonstration that extrachromosomal circular DNA (eccDNA), which is produced during apoptosis of eukaryotic cells or produced synthetically, is an immunostimulant. As described herein, eccDNA may be used to elicit, enhance, prolong, or modulate an immune response in an individual, such as a human, including innate and adaptive immune responses. In some embodiments, eccDNA alone is used to elicit, enhance, prolong, or modulate immunity/an immune response. In some embodiments, eccDNA is administered in combination with an antigen and optionally an adjuvant for stimulating immunity, such as in a vaccine. In some aspects, eccDNA and formulations thereof are used in the treatment or prophylaxis of a disease involving the immune system, such as an infectious disease, a cancer, or an allergy.

Extrachromosomal Circular DNA (eccDNA)

The methods and compositions described herein are based, at least in part, on the demonstration that extrachromosomal circular DNA (“eccDNA”), a circular, chromosomally derived, double stranded DNA molecule that is produced during apoptosis in many eukaryotic species, is capable of stimulating (stimulates) an immune response in an individual to whom it is administered (is immunostimulatory). Accordingly, eccDNA may be produced, collected, and administered to an individual to stimulate an immune response in that individual. This feature of eccDNA can be used for a variety of therapeutic applications, including the development of approaches and compositions for improving immune responses to a wide range of antigens that individuals encounter, including those in administered in vaccines.

For several decades, the occurrence of eccDNA has been described in numerous eukaryotic species, including numerous animal, plant, and yeast species. Depending on its size, eccDNA has been referred to in the art in a variety of ways, including double minutes (DMs) and extrachromosomal DNA (ecDNA) for eccDNA with a size ranging from ˜100 kb to >1 mb, as well as microDNA and small polydisperse circular DNA (spcDNA) for eccDNA with a size of as few as a few hundred bases. Suggested functions in the art for eccDNA range from involvement in the repair of DNA damage, hyper-transcription, homologous recombination, stress during replication, gene expression during cancer, and aging. However, how eccDNA is produced was not understood until recently.

Without wishing to be bound by theory, eccDNA is endogenously generated as a byproduct of apoptosis (programed cell death) in various multicellular eukaryotic organisms, such as those belonging to the kingdom Animalia, including humans. During apoptosis, cells activate a tightly regulated signaling pathway mediated by caspase proteins in response to internal or external stimuli. Caspase-mediated apoptosis leads to numerous changes in cells, including the dissolution of nuclei and fragmentation of the chromosomes comprising the genome. Apoptotic DNA fragmentation (ADF) is catalyzed by caspase-activated DNase (CAD; alternately known as DNA fragmentation factor (DFF)), DNase γ, or Endonuclease G, which specifically cleaves chromosomal DNA between nucleosomes, producing chromosomal DNA fragments in oligonucleosomal sizes. These fragmented chromosomal DNA may be subsequently reattached by a DNA ligase to produce a circular DNA molecule that comprises sequence originating from one or more chromosomes. This DNA species is referred to as eccDNA. In humans, eccDNA may be produced by the activity of any one of the endogenously expressed human DNA ligases, Lig1, Lig3, Lig4, which overlap in their function.

Some aspects of the disclosure pertain to the collection (separation), enrichment, analysis, or administration of eccDNA, or any combination thereof. In some embodiments, the eccDNA is derived from a eukaryote. In some embodiments, the eccDNA is derived from a mammal. In some embodiments, the eccDNA is derived from a human. In some embodiments, the eccDNA is essentially free of prokaryotic (bacterial), bacteriophage, plasmid, and cosmid sequences. In some embodiments, the eccDNA is essentially free of site-specific recombination sites (e.g., Cre recombinase sites, FLP-FRT recombinase sites). In some embodiments, the eccDNA is non-replicating. In some embodiments, the eccDNA does not contain an origin of replication. In some embodiments, the eccDNA comprises one or more genes. In some embodiments, the eccDNA does not comprise genes or does not comprise genes which can be expressed from the eccDNA or are capable of being expressed from the eccDNA (e.g., the gene or genes are not functionally linked to a promotor sequence). In some embodiments, the eccDNA has a minimum size (length) of at least about 66 nucleotides, of at least about 75 nucleotides, at least about 100 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 1250 nucleotides, at least about 1500 nucleotides, at least about 1750 nucleotides, or at least about 2000 nucleotides. In some embodiments, the eccDNA has a maximum size (length) of about 200 nucleotides, about 300 nucleotides, about 400 nucleotides, about 500 nucleotides, about 600 nucleotides, about 700 nucleotides, about 800 nucleotides, about 900 nucleotides, about 1000 nucleotides, about 1250 nucleotides, about 1500 nucleotides, about 1750 nucleotides, about 2000 nucleotides, about 2250 nucleotides, about 2500 nucleotides, about 2750 nucleotides, or about 3000 nucleotides. In some embodiments, the eccDNA ranges in size from about 66 to about 3000 nucleotides, from about 100 to about 2500 nucleotides, or from about 200 to about 2000 nucleotides. As used herein, the terms “nucleotides” (nt) and “base pairs” (bp) may be used interchangeably to refer to the size (length) of double stranded DNA (e.g., eccDNA). Where the term “nucleotides” is used to refer to the size (length) of a double stranded DNA (e.g., eccDNA), the term should be understood to indicate the size for each strand of the double stranded DNA.

Methods for Enrichment of eccDNA

In certain aspects, the present invention relates to methods for enriching eccDNA present in a sample that comprises a mixture of DNA species, such as circular DNA and linear DNA, wherein the circular DNA in the mixture is at least partially eccDNA. In some embodiments, eccDNA is enriched from a sample that is a biological sample. As used herein, the term “biological sample” refers to any sample that contains material which is produced by an organism (e.g., a living organism or a dead organism). A biological sample may comprise cells. In some embodiments, eccDNA is enriched from a biological sample obtained from a population of cells, such as, but not limited to, a population of cultured cells. In some embodiments, eccDNA is enriched from a biological sample that is obtained from an individual (e.g., a subject or a patient, such as a human or other animal). In some embodiments, eccDNA is enriched from a liquid biological sample (i.e., a biological fluid) that is obtained from an individual, such as, but not limited to, blood, plasma, mucus, sputum, saliva, urine, lymph, or cerebrospinal fluid. In some embodiments, eccDNA is enriched from a solid biological sample obtained from an individual, such as, but not limited to, a tissue biopsy. A sample or mixture comprising eccDNA that has not been enriched (unenriched eccDNA) is referred to as an unenriched sample.

In some embodiments, cells in a biological sample may be lysed prior to enrichment of eccDNA by any technique known to those of skill in the relevant art. In such embodiments, cells in a sample are lysed under conditions at which eccDNA is irreversibly denatured or fragmented at a lower rate than other DNA species present in the sample (e.g., linear DNA), or preferably under conditions at which eccDNA is not irreversibly denatured or fragmented. Cells may be lysed, for example through the use of mechanical means, such as sonication or agitation with glass beads, through the use of chemical means, such as alkaline lysis, or through a combination of techniques thereof. In embodiments where cells in a provided sample are lysed by means of alkaline lysis, said lysis employs a buffered alkaline solution having a pH between 11.0 and 12.3, optimally having a pH of 11.8, as opposed to a conventional unbuffered alkaline solution (e.g., 0.2 M NaOH), in order to prevent damage to eccDNA in the sample during lysis.

In some embodiments, cells in a sample may be fixed prior to lysis, such as, for example, by means of methanol fixation or another method of fixation that is known in the art. In some embodiments, cells in a solid biological sample, such as, for example, a tissue sample, may be subjected to cell dissociation or tissue grinding prior to lysis by one or more chemical means, such as alkaline lysis (e.g., buffered alkaline lysis).

In some embodiments, cells in a sample are placed in a suspension buffer prior to lysis. In some embodiments, the suspension buffer comprises a chelating agent, such as, for example, ethylenediaminetetraacetic acid (EDTA) or another chelating agent that is known in the art. In some embodiments, the suspension buffer comprises 0-100 mM chelating agent, such as 0-100 mM EDTA, preferably 0-20 mM EDTA, or optimally 10 mM EDTA. In some embodiments, the suspension buffer comprises an enzyme that degrades RNA, such as, for example, RNase A or another RNA-degrading enzyme that is known in the art. In some embodiments, the suspension buffer comprises 10-300 mg/mL of an enzyme that degrades RNA, such as 10-300 mg/mL RNase A, preferably 50-150 mg/mL RNase A, or optimally 110 mg/mL RNase A. In some embodiments, the suspension buffer comprises a salt, such as, for example, NaCl, KCl, or another halide salt that is known in the art. In some embodiments, the suspension buffer comprises 100-200 mM salt, such as 100-200 mM NaCl, or optimally 150 mM NaCl. In some embodiments, the suspension buffer comprises glycerol, such as, for example, 1% glycerol. In some embodiments, the suspension buffer comprises a reducing agent, such as, for example, 2-mercaptoethanol (β-mercaptoethanol; BME), 1,4-dithiothreitol (DTT), tris(2-carboxyethyl)phosphine (TCEP), or another reducing agent that is known in the art. In some embodiments, the suspension buffer comprises 0.2% reducing agent, such as 0.2% 2-mercaptoethanol. In some embodiments, the suspension buffer has a neutral pH, such as, for example, a pH between 6.0 and 8.0.

In some embodiments, cells in a sample are lysed by addition of a buffered alkaline lysis solution. In some embodiments, the buffered alkaline lysis solution comprises a compound having an acid dissociation pH (pKa, i.e., the pH at which the compound deprotonates) between 11.0 and 12.3, such as, for example, pyrrolidine, piperidine, glucose, arginine, lysine, or another compound known in the art that has a pKa between 11.0 and 12.3. In some embodiments, the buffered alkaline lysis solution comprises 0.01-3.0 M of a compound having a pKa between 11.0 and 12.3. In some embodiments, the buffered alkaline lysis solution comprises 0.1-1.5 M pyrrolidine, or optimally 0.5 M pyrrolidine. In some embodiments, the buffered alkaline lysis solution comprises a chelating agent, such as, for example, EDTA or another chelating agent that is known in the art. In some embodiments, the buffered alkaline lysis solution comprises 0-100 mM of a chelating agent, such as 0-100 mM EDTA, preferably 5-30 mM EDTA, or optimally 20 mM EDTA. In some embodiments, the buffered alkaline lysis solution comprises a detergent, such as, for example, sodium dodecyl sulphate (SDS) or another detergent that is known in the art. In some embodiments, the buffered alkaline lysis solution comprises 0.1%-5% detergent, such as 0.1-5% SDS, preferably 0.5-2% SDS, or optimally 1% SDS. In some embodiments, the buffered alkaline lysis solution comprises a reducing agent, such as, for example, 2-mercaptoethanol, DTT, TCEP, or another reducing agent that is known in the art. In some embodiments, the buffered alkaline lysis solution comprises 0.2% reducing agent, such as 0.2% 2-mercaptoethanol. In some embodiments, the buffered alkaline lysis solution has a pH between 11.0 and 12.3, preferably between 11.3 and 12.0, or optimally 11.8.

In some embodiments, a sample comprising a buffered alkaline solution is subsequently neutralized by the addition of a neutralization solution that reduces the alkalinity of the sample. In some embodiments, the neutralization solution comprises a neutralizing agent, such as potassium acetate (KOAc) or an alternate neutralizing agent known in the art, such as another potassium salt. In some embodiments, the neutralization solution comprises 0.5-5.0 M neutralizing agent, such as 0.5-5.0 M KOAc, preferably 1.0-3.0 M KOAc, or optimally 2.0 M KOAc. In some embodiments, the neutralization solution has a pH less than 6.0, preferably 5.5.

In some embodiments, a sample comprising a neutralized buffered alkaline solution is subsequently treated with a solution comprising a DNA extraction agent, such as, for example, cetyl trimethylammonium bromide (CTAB), or another DNA extraction agent that is known in the art. In some embodiments, the sample comprising a neutralized buffered alkaline solution is subsequently treated with a solution comprising 0.1%-3% CTAB. In some embodiments, the sample comprising a neutralized buffered alkaline solution is subsequently treated with a solution comprising 0.2-3.0 M salt, such as for example 0.2-3.0 M NaCl, or another halide salt known in the art.

In some embodiments, circular DNA species from lysed cells in a sample are extracted using a binding medium, such as, for example, a silica column or silica beads (e.g., magnetic silica beads), or another binding medium that is known in the art. In some embodiments, the binding medium to which circular DNA species are bound is washed with a wash solution prior to elution of the bound circular DNA. In some embodiments, the wash solution comprises a solvent in which circular DNA has relatively low solubility, such as, for example, ethanol or another alcohol known in the art (e.g., isopropanol). In some embodiments, the wash solution comprises 50-90% ethanol. In some embodiments, the bound circular DNA species are eluted from the binding medium using an elution buffer in which circular DNA has relatively high solubility, such as, for example, sterile water or another suitable buffer that is generally known in the art.

In some embodiments, following lysis of cells in a sample, circular DNA species in the sample are treated with an agent that selectively linearizes circular DNA species that are distinct from eccDNA. In such embodiments, the agent linearizes eccDNA to a lesser extent than it linearizes other circular DNA species present in the provided sample, or preferably does not linearize eccDNA. In some embodiments, other circular DNA species in a provided sample which are not eccDNA comprise mitochondrial DNA (mtDNA).

In some embodiments, the agent is an enzyme. In some embodiments the agent is a restriction enzyme. In embodiments where the agent is a restriction enzyme, the agent may be any restriction enzyme that targets a restriction site that occurs more often (is more common) in mtDNA sequences than in eccDNA sequences, where eccDNA sequences are understood to be derived from the genome (e.g., any one or more chromosomes, such as any one or more of the 23 human chromosomes). In some embodiments, the agent is a restriction enzyme that targets a restriction site that is relatively rare within the target sequences (e.g., a “rare cutter” restriction enzyme), such as PacI, which targets sites having a sequence of 5′ . . . TTAAT/TAA . . . 3′ and is readily available through a variety of commercial sources along with optimized protocols for its use (e.g., New England Biolabs #R0547; ThermoFisher Scientific #ER2201). In some embodiments, the agent is a restriction enzyme that is not PacI (e.g., a different “rare cutter” restriction enzyme) and selectively linearizes circular DNA that is not eccDNA (e.g., mtDNA), as may be readily identified by one of ordinary skill in the art.

In some embodiments, the agent is an enzyme that is not a restriction enzyme. In some embodiments, the agent is a CRISPR-guided nuclease (e.g., Cas9) comprising a guide RNA complimentary to a sequence that is specific for circular DNA species in a provided sample that are not eccDNA (e.g., mtDNA). In such embodiments, the CRISPR-guided nuclease (e.g., Cas9) may be used to induce double strand breaks in (cleave) the target sequence(s). Numerous techniques for specifically targeting a sequence (e.g., a mtDNA sequence) with CRISPR by designing and providing a guide RNA complimentary to the target sequence are known in the art, for example, in Sander J D, and Joung J K. “CRISPR-Cas systems for editing, regulating and targeting genomes.” Nat Biotechnol. 2014 April; 32(4): 347-355., which is expressly incorporated by reference herein in its entirety.

In some embodiments, following linearization of circular DNA species in a provided sample which are not eccDNA (e.g., linearized mtDNA), the sample is treated with an agent that selectively digests (hydrolyzes) linear DNA species. In such embodiments, the agent digests eccDNA to a lesser extent than it digests linear DNA in the provided sample, or preferably does not digest eccDNA. In some embodiments, the agent is an enzyme that does not digest nicked or closed-circular double stranded DNA. In some embodiments, the agent is plasmid safe DNase, which is commercially available along with optimized protocols for its use (Lucigen #E3101K). In some embodiments, the agent is Exonuclease V, which is commercially available along with optimized protocols for its use (New England Biolabs #M0345). In some embodiments, the agent is an enzyme that is not plasmid safe DNase or Exonuclease V and selectively digests linear DNA (e.g., linearized mtDNA), as may be readily identified by one of ordinary skill in the art.

In some embodiments, the linearization of circular DNA species that are not eccDNA and the digestion of linear DNA species occur sequentially. In some embodiments, the linearization of circular DNA species that are not eccDNA and the digestion of linear DNA species occur concurrently.

In some embodiments, following the digestion of linearized DNA species in a provided sample, eccDNA is physically separated from other DNA species in the sample, such as linear DNA (e.g., digested linearized mtDNA). In such embodiments, eccDNA may be separated from digested linear DNA species by any means for separating distinct DNA species that is known in the art, such as separation by size, in order to produce enriched eccDNA. For example, eccDNA may be separated from digested linear DNA through the use of silica (e.g., silicon dioxide (SiO₂)) beads or resins, as are known in the art and readily available (e.g., ThermoFischer Scientific #K0513; Qiagen #12125). Alternately, eccDNA may be separated from linear DNA through the use of another DNA-binding medium, such as cellulose (see, e.g., Moeller J R, et al. “Paramagnetic cellulose DNA isolation improves DNA yield and quality among diverse plant taxa.” Appl Plant Sci. 2014 Oct. 2; 2(10):apps.1400048, which is incorporated by reference herein). Separation of eccDNA may also be carried out through the use of magnetic beads, such as magnetic silica beads (e.g., Fe₃O₄ magnetic beads coated with a silicon dioxide (SiO₂) layer) or magnetic cellulose beads, as are known in the art and readily available (e.g., Zymo Research #D4100-2; Cytiva Life Sciences #29357369; Bioneer #TS-1010). In some embodiments, eccDNA may be alternately separated though any variety of chemical means known in the art, such as, but not limited to, ion exchange, phenol chloroform extraction, and/or ethanol precipitation. In some embodiments, eccDNA may be separated by phenol/chloroform/isoamyl alcohol extraction (e.g., treatment with 25:24:1 phenol:chloroform:isoamyl alcohol, followed by centrifugation, addition of ethanol to precipitate eccDNA, storage at −80° C., and removal of residual solvent).

In some embodiments, the purity of the separated (enriched) eccDNA is further improved by a second round of separation that reduces the amount of co-enriching linear DNA, such as, for example, an additional round of separation by selectively binding eccDNA to silica beads or resin. In some embodiments, the purity of the separated (enriched) eccDNA is further improved by adding to the eccDNA a solution that selectively enhances the recovery of eccDNA via binding to magnetic silica beads. In some embodiments, the solution comprises a chaotropic agent, such as, for example, guanidine thiocyanate or another chaotropic agent that is known in the art. In some embodiments, the solution comprises 0.2-6.0 M guanidine thiocyanate. In some embodiments, the solution comprises phenol, preferably 1%-80% phenol. In some embodiments, the solution comprises spermidine or spermine, preferably 0.1 μM-500 mM spermidine or spermine. In some embodiments the solution comprises 21examine cobalt (III) chloride, preferably 0.1 μM-500 mM 2lexamine cobalt (III) chloride. In some embodiments, magnetic silica beads bound to eccDNA are washed with a high salt solution, preferably 1.0-4.0 M NaCl or another halide salt that is known in the art. In some embodiments, the high salt solution comprises 0-50% ethanol or another alcohol that is known in the art (e.g., isopropanol). In some embodiments, eccDNA bound to the magnetic silica beads are eluted from the magnetic silica beads using an elution buffer in which eccDNA has relatively high solubility, such as, for example, sterile water, 10 mM Tris-Cl (pH 8.5), or another suitable buffer that is generally known in the art. In some embodiments, after separation the enriched eccDNA is essentially free of linear DNA species.

In some embodiments, the amount of resulting enriched eccDNA may be increased by subsequent DNA amplification. The amount of enriched eccDNA may be increased by any enzymatic or non-enzymatic isothermal amplification technique that is generally known in the art. In some embodiments, the amount of enriched eccDNA is amplified (increased) by rolling circle amplification (RCA), which is well known in the art for the amplification of circular DNA. Briefly, RCA reactions may be used to produce amplified eccDNA by combining a DNA polymerase suitable for RCA (e.g., Phi29 (New England Biolabs #M0269), Bst DNA polymerase (New England Biolabs #M0275), Deep Vent DNA polymerase (New England Biolabs #M0258)), oligonucleotide primers (e.g., a set of random oligonucleotide primers, e.g., ThermoFisher Scientific #S0181), eccDNA templates, free deoxyribonucleotide triphosphates (dNTPs), and a buffer suitable for RCA. RCA reactions are typically isothermal (conducted at a single temperature). Additional information regarding techniques for amplifying circular DNA species by RCA may be found, for example, in Bhat A I and Rao G P (2020) “Rolling Circle Amplification (RCA).” In: Characterization of Plant Viruses. Springer Protocols Handbooks. Humana, New York, NY; Detter J, et al. (2004). “Phi29 DNA polymerase based rolling circle amplification of templates for DNA sequencing.” Lawrence Berkeley National Laboratory. LBNL Report #: LBNL-54644; Stevens H, et al “Multiply primed rolling-circle amplification method for the amplification of circular DNA viruses.” Cold Spring Harb Protoc. 2010 April; 2010(4):pdb.prot5415, which are expressly incorporated by reference herein in their entirety.

In some embodiments, a method provided herein for enriching eccDNA present in a sample comprising a mixture of DNA species may be conducted by linearizing non-eccDNA circular DNA, digesting linear DNA, and separating enriched eccDNA as a set of sequential steps. In some embodiments a method provided herein for enriching eccDNA present in a sample comprising a mixture of DNA species may be conducted by performing one or more of the steps described herein (e.g., linearizing, digesting, separating) as concurrent steps.

Methods for Assessing Levels of eccDNA in an Individual

In certain aspects, the present invention relates to methods for assessing the level of eccDNA in an individual, such as an individual who is known, suspected to have, or is at risk for a disease associated with an increase in the level of eccDNA. In some embodiments, the disease associated with an increase in the level of eccDNA is a disease that is associated with an increase in apoptosis in cells of one or more tissues. In some embodiments, the disease is a disease caused by one or more pathogens (e.g., bacteria, viruses, or parasites), a neurodegenerative disease, an inflammatory disease, or an autoimmune disease. In some embodiments, the disease is sepsis, acute respiratory distress syndrome (ARDS), CAR T cell-induced cytokine release syndrome (CRS), coronavirus disease 2019 (COVID-19), Parkinson's disease, Huntington's disease, Alzheimer's disease, Lou Gehrig's disease, Type I diabetes, autoimmune thyroid disease (e.g., Graves' disease), systemic lupus erythematosus, rheumatoid arthritis, or Sjogren's syndrome.

In some embodiments, a method for determining the state of a disease associated with an increased level of eccDNA in an individual comprises obtaining, from the individual, a sample comprising eccDNA from the individual, measuring the level of eccDNA in the sample, and comparing the level of eccDNA in the sample with a reference level of eccDNA. In some embodiments, a method for determining the state of a disease associated with the presence of eccDNA in an individual comprises obtaining, from the individual, a sample from the individual and determining if eccDNA is present in the sample. In some embodiments, the sample is a liquid biological sample (i.e., a biological fluid) that is obtained from the individual, such as, but not limited to, blood, plasma, mucus, sputum, saliva, urine, lymph, or cerebrospinal fluid. In some embodiments, the sample is a solid biological sample obtained from the individual, such as, but not limited to, a tissue biopsy. In some embodiments, the eccDNA in the sample is enriched prior to measurement, for example, by use of one of the methods disclosed herein (e.g., by treating with an enzyme that linearizes mtDNA, treating with an enzyme that digests linear DNA, and separating eccDNA). In some embodiments, the eccDNA in the sample is amplified by RCA prior to measurement. In embodiments where the eccDNA in the sample is amplified by RCA prior to measurement and the initial amount of eccDNA in the sample was too low to reliably detect, the initial amount of eccDNA in the sample may be inferred based on the final amount obtained by RCA and the conditions under which RCA was conducted (e.g., polymerase type, polymerase concentration, primer concentration, dNTP concentration, reaction temperature, reaction duration).

In embodiments where the level of eccDNA in the sample is measured, the level of eccDNA may be measured by any means suitable for quantifying DNA, such as, but not limited to, spectrophotometric analysis, quantitative ethidium bromide gel electrophoresis, or enzyme-linked immunosorbent assay (ELISA), including polymerase chain reaction (PCR)-ELISA, as are well known in the art. Details for such techniques may be found for example in Barbas C F 3rd, et al. “Quantitation of DNA and RNA.” CSH Protoc. 2007 Nov. 1; 2007:pdb.ip47; and Sue M J, et al. “Application of PCR-ELISA in molecular diagnosis.” Biomed Res Int. 2014; 2014:653014, which are expressly incorporated by reference herein in their entirety.

In some embodiments, the reference level of eccDNA to which the level of eccDNA measured in the sample is compared is a reference level of eccDNA that is indicative of disease. In some embodiments, the reference level of eccDNA to which the level of eccDNA measured in the sample is compared, is a level of eccDNA measured in corresponding sample obtained from an individual known to have the disease, or a mean level of eccDNA measured in a set of corresponding samples obtained from a set of individuals known to have the disease. In some embodiments, the individual for whom the state of a disease associated with an increase in the level of eccDNA is assessed is identified as having the disease if the level of eccDNA measured in the sample does not statistically differ from the reference level indicative of disease. In such embodiments, the individual may be subsequently administered one or more therapies for treatment of the disease.

In some embodiments, the reference level of eccDNA to which the level of eccDNA measured in the sample is compared is a reference level of eccDNA that is not indicative of disease. In some embodiments, the reference level of eccDNA to which the level of eccDNA measured in the sample is compared, is a level of eccDNA measured in corresponding sample obtained from an individual known to be free of the disease (healthy), or a mean level of eccDNA measured in a set of corresponding samples obtained from a set of individuals known to be free of the disease. In some embodiments, the individual for whom the state of a disease associated with an increase in the level of eccDNA is assessed is identified as having the disease if the level of eccDNA measured in the sample is statistically increased compared to that of the reference level indicative of being free of the disease. In such embodiments, the individual may be subsequently administered one or more therapies for treatment of the disease. In some embodiments, the individual for whom the state of a disease associated with an increase in the level of eccDNA is assessed is identified as being free of the disease if the level of eccDNA measured in the sample does not statistically differ from that of the reference level indicative of being free of the disease.

In embodiments where the level of eccDNA measured in a sample obtained from an individual is compared to a reference level indicative of the presence or absence of a disease, such a comparison may be made using any suitable statistical model that is readily known by one skilled in the relevant art. The level of eccDNA measured in a sample obtained from an individual may be compared to a reference level of t-statistic eccDNA by, for example, statistical hypothesis testing.

### Methods for In Vitro Synthesis of Circular DNA Species

Certain aspects of the present invention relate to circular DNA species that are functionally equivalent to eccDNA (e.g., are equally immunostimulatory when administered to an individual). In some embodiments, circular DNA species are contemplated that are entirely or partially derived from the chromosome of an organism comprising a genome consisting of circular DNA, such as a prokaryote, an archaea, or a circular DNA virus. In some embodiments, circular DNA species are contemplated that are entirely or partially derived from a plasmid or cosmid. In some embodiments, circular DNA species are contemplated that are entirely or partially derived from one or more chromosomes of an organism comprising a genome consisting of linear DNA, such as a eukaryote or a linear DNA virus. In some embodiments, circular DNA species are contemplated that comprise one or more random sequences (e.g., a sequence that is randomly synthesized). In some embodiments, circular DNA species are contemplated that comprise an entirely random sequence. In some embodiments, circular DNA species are contemplated that are essentially free of prokaryotic (bacterial), bacteriophage, plasmid, and cosmid sequences, are essentially free of site-specific recombination sites (e.g., Cre recombinase sites, FLP-FRT recombinase sites), are non-replicating, do not comprise an origin of replication, do not comprise genes, and/or do not comprise genes which are functionally linked to a promotor sequence, or any combination thereof. In some embodiments, circular DNA species are contemplated that range in size (length) from about 66 nucleotides to about 3000 nucleotides. In some embodiments, circular DNA species are contemplated that range in size (length) from about 200 nucleotides to about 2000 nucleotides.

In some embodiments, circular DNA species contemplated herein are synthetically produced. In some embodiments, circular DNA species contemplated herein are synthetically produced by means of ligation-assisted minicircle accumulation (LAMA). Techniques for conducting LAMA are known in the art, such as, for example, in Du, Quan et al. “Kinking the double helix by bending deformation.” Nucleic Acid Res. 2008 March; 36(4):1120-8, which is expressly incorporated by reference herein in its entirety. Briefly, LAMA may be used to produce circular DNA species by first preparing reactions comprising a forward and reverse single stranded linear DNA template comprising the sequence of the final circular DNA produce, two pairs of oligonucleotide primers having a 5′ phosphate group, dNTPs, and a suitable DNA polymerase (e.g., Q5 DNA polymerase) in a suitable buffer, and then conducting polymerase chain reaction to prepare amplified template sequences having 5′ phosphate groups. Second, circular DNA species are synthesized by preparing reactions comprising template sequences having 5′ phosphate groups and a suitable DNA ligase (e.g., HiFi Taq DNA ligase) in a suitable buffer, then cycling the temperature of the reactions to produce circularly annealed templates that are subsequently ligated into a closed circle.

In some embodiments, the sequence of a DNA species produced by LAMA is a random sequence. In some embodiments, the sequence of the single stranded linear DNA templates for LAMA are arbitrarily selected from the set of possible sequences having a defined length and overall proportion of guanine and cytosine residues (i.e., % GC content). In some embodiments, custom DNA templates for the synthesis of circular DNA species through LAMA are readily available for synthesis from a range of commercial sources (e.g., Integrated DNA Technologies).

In some embodiments, the sequence of a circular DNA species produced by LAMA may contain one or more modified nucleotides. In some embodiments, the modified nucleotides confer additional stability or functionalization to a circular DNA species. In some embodiments, one or more modified nucleotides are introduced to the sequence of a circular DNA species prior to LAMA (e.g., by modifying one or more single stranded template sequences). In some embodiments, one or more modified nucleotides are introduced to the sequence of a circular DNA species after LAMA (e.g., by enzymatically modifying the circular DNA species after ligation). In some embodiments, the sequence of a circular DNA species produced by LAMA may contain one or more of the following modified nucleotides: deoxyinosine (dI), deoxyuracil (dU), a nucleotide modified with a spacer (e.g., spacer C3, spacer C6, spacer C12, spacer 9, spacer 18, 1′,2′-Dideoxyribose (dSpacer)), or a nucleotide bearing another modification (e.g., NH₂ dT, biotin dT, Cy3 dT, Cy5 dT, TAMRA dT, ROX dT, FAM dT, HEX, dT, Dig dT, BHQ1 dT, BHQ1 dT, Ferrocene dT). In some embodiments, the sequence of a circular DNA species produced by LAMA may contain one or more nucleotides linked by a phosphorothioate linkage.

### Immunostimulatory Compositions and Vaccines

Compositions Comprising eccDNA

Compositions comprising one or more immunostimulatory circular DNA species of the present disclosure are contemplated. In some embodiments, compositions (e.g., pharmaceutical compositions) of the present disclosure comprise immunostimulatory eccDNA. In some embodiments, compositions (e.g., pharmaceutical compositions) of the present disclosure comprise a circular DNA species that is functionally equivalent to immunostimulatory eccDNA (e.g., with respect to its ability to stimulate an immune response). In some embodiments, compositions (e.g., pharmaceutical compositions) of the present disclosure comprise one or more nucleic acids (e.g., linear DNA, RNA) in addition to immunostimulatory eccDNA or a circular DNA that is functionally equivalent to immunostimulatory eccDNA. In some embodiments, compositions (e.g., pharmaceutical compositions) of the present disclosure (a) comprise immunostimulatory eccDNA or a circular DNA that is functionally equivalent to eccDNA and (b) are essentially free of other nucleic acids (e.g., linear DNA, RNA). A composition that comprises immunostimulatory eccDNA or a circular DNA species that is functionally equivalent to eccDNA (eccDNA equivalent) is an “immunostimulatory” composition.

An “immunostimulatory” circular DNA or composition alters (elicits, enhances, prolongs, or modulates) an immune response in an individual (e.g., a mammalian subject such as a human) to which it is administered. An “immune response” is a response by a cell of the immune system, such as an antigen-presenting cell, dendritic cell, monocyte, macrophage, NKT cell, NK cell, basophil, eosinophil, or neutrophil, B cell, T cell (CD4 or CD8), regulatory T cell, antigen-presenting cell, dendritic cell, monocyte, macrophage, NKT cell, NK cell, basophil, eosinophil, or neutrophil, to a stimulus (e.g., to eccDNA, a functionally equivalent circular DNA, or an antigen).

In some embodiments, the immune response stimulated by an immunostimulatory composition is specific for a particular antigen (an “antigen-specific response” or “adaptive immune response”) and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some embodiments, the immune response stimulated by an immunostimulatory composition is not specific for a particular antigen.

In some embodiments, an antigen-specific immune response includes both a humoral and/or a cell-mediated immune response to an antigen. A “humoral immune response” is an antibody-mediated immune response and involves the induction and generation of antibodies that recognize and bind with some affinity for an antigen, while a “cell-mediated immune response” is one mediated by T-cells and/or other white blood cells. A “cell-mediated immune response” is elicited by the presentation of antigenic epitopes in association with Class I or Class II molecules of the major histocompatibility complex (MHC), CD1 or other non-classical MHC-like molecules. This activates antigen-specific CD4+T helper cells or CD8+ cytotoxic lymphocyte cells (“CTLs”). CTLs have specificity for peptide antigens that are presented in association with proteins encoded by classical or non-classical MHCs and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide or other antigens in association with classical or non-classical MHC molecules on their surface. A “cell-mediated immune response” also refers to the production of cytokines, chemokines and other such molecules produced by activated T-cells and/or other white blood cells, including those derived from CD4+ and CD8+ T-cells. The ability of an immunostimulatory composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized individual, or by measurement of cytokine production by T cells in response to re-stimulation with antigen. Such assays are well known in the art. See, e.g., Erickson et al. (1993) J. Immunol. 151:4189-4199; and Doe et al. (1994) Eur. J. Immunol. 24:2369-2376, which are expressly incorporated herein in their entirety.

In some embodiments, the immune response stimulated by the immunostimulatory compositions is an innate immune response. An “innate immune response” refers to the response by the innate immune system. The innate immune system uses a set of germline-encoded receptors (“pattern recognition receptor” or “PRR”) for the recognition of conserved molecular patterns present in microorganisms. These molecular patterns occur in certain constituents of microorganisms including: lipopolysaccharides, peptidoglycans, lipoteichoic acids, phosphatidyl cholines, bacteria-specific proteins, including lipoproteins, bacterial DNAs, viral single and double stranded RNAs, unmethylated CpG-DNAs, mannans and a variety of other bacterial and fungal cell wall components. Such molecular patterns can also occur in other molecules such as plant alkaloids. These targets of innate immune recognition are called Pathogen Associated Molecular Patterns (PAMPs) since they are produced by microorganisms and not by the infected host organism. In some embodiments, the innate immune response stimulated by the immunostimulatory compositions confers heterologous (“non-specific”) immunity to a broad range of pathogenic microbes by enhancing innate immune responses to subsequent stimuli, a phenomenon known as “trained immunity”, a form of innate memory, e.g., as described in Netea et al. (Trained Immunity: An Ancient Way of Remembering. Cell Host Microbe. 2017 Mar. 8; 21(3):297-300, incorporated herein by reference).

The receptors of the innate immune system that recognize PAMPs are called Pattern Recognition Receptors (PRRs). (Janeway et al. (1989) Cold Spring Harb. Symp. Quant. Biol. 54: 1-13; Medzhitov et al. (1997) Curr. Opin. Immunol. 94: 4-9, incorporated herein by reference). PRRs vary in structure and belong to several different protein families. Some of these receptors recognize PAMPs directly (e.g., CD14, DEC205, collectins), while others (e.g., complement receptors) recognize the products generated by PAMP recognition. Members of these receptor families can, generally, be divided into three types: 1) humoral receptors circulating in the plasma; 2) endocytic receptors expressed on immune-cell surfaces, and 3) signaling receptors that can be expressed either on the cell surface or intracellularly. (Medzhitov et al. (1997) Curr. Opin. Immunol. 94: 4-9; Fearon et al. (1996) Science 272: 50-3, incorporated herein by reference). Non-limiting examples of PRRs include: Toll-like receptors (e.g., TLR2, TLR4, TLR9), NOD1/2, RIG-1/MDA-5, C-type lectins, and STING. In some embodiments, immunostimulatory compositions comprising eccDNA or a functionally equivalent circular DNA elicit an innate immune response by activating the stimulator of interferon genes (STING) pathway, an intracellular sensor of both self and foreign double stranded DNA.

Cellular PRRs are expressed on effector cells of the innate immune system, including cells that function as professional antigen-presenting cells (APC) in adaptive immunity. Such effector cells include, but are not limited to, macrophages, dendritic cells, B lymphocytes and surface epithelia. This expression profile allows PRRs to directly induce innate effector mechanisms, and also to alert the host organism to the presence of infectious agents by inducing the expression of a set of endogenous signals, such as inflammatory cytokines and chemokines, including, without limitation: chemokines, interferons, interleukins, lymphokines, and tumor necrosis factors. This latter function allows efficient mobilization of effector forces to combat the invaders.

In some embodiments, immunostimulatory compositions comprising immunostimulatory eccDNA or a functionally equivalent circular DNA induce the production of various cytokines and/or chemokines. It is demonstrated herein that eccDNA or a functionally equivalent circular DNA alone is sufficient to induce cytokine and/or chemokine production (e.g., IFNα, IFNβ, IFNγ, IL-6, TNFα).

In some embodiments, immunostimulatory compositions comprising immunostimulatory eccDNA or a functionally equivalent circular DNA comprise one or more eccDNAs or functionally equivalent (immunostimulatory) circular DNAs. In some embodiments, immunostimulatory compositions comprising eccDNA or a functionally equivalent circular DNA comprise one or more eccDNAs or functionally equivalent circular DNAs that range in size (length) from about 66 nucleotides to about 3000 nucleotides. In some embodiments, immunostimulatory compositions comprising eccDNA or a functionally equivalent circular DNA comprise one or more eccDNAs or functionally equivalent circular DNAs that range in size (length) from about 70 nucleotides to about 2000 nucleotides. In some embodiments, immunostimulatory compositions comprising eccDNA or a functionally equivalent circular DNA comprise one or more eccDNAs or functionally equivalent circular DNAs that range in size (length) from about 200 nucleotides to about 2000 nucleotides.

Compositions Comprising eccDNA and Antigens

In some embodiments, an immunostimulatory composition comprises eccDNA or a functionally equivalent circular DNA and an antigen. An “antigen” refers to an entity that is bound by an antibody or receptor, or an entity that induces the production of the antibody. In some embodiments, an antigen increases the production of antibodies that specifically bind the antigen when administered to an individual. In some embodiments, an antigen comprises a protein or polypeptide, e.g., an “immunostimulatory polypeptide”. In some embodiments, the term “antigen” encompasses nucleic acids (e.g., DNA or RNA molecules) that encode immunogenic polypeptides. In some embodiments, the antigen is from a microbial pathogen. For example, the antigen may comprise parts (coats, capsules, cell walls, flagella, fimbriae, and toxins) of bacteria, viruses, fungi, and other microorganisms. In some embodiments, the antigen is a cancer-specific antigen.

In some embodiments, a protein or polypeptide antigen is a wild type protein or polypeptide. In some embodiments, a protein or polypeptide antigen is a polypeptide variant to a wild type protein or polypeptide. The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, polypeptide variants possess at least 50% identity to a native or reference sequence. In some embodiments, variants share at least 70%, at least 80%, at least 90%, at least 95%, or at least 99% identity with a native or reference sequence. In some embodiments, a polypeptide variant comprises substitutions, insertions, deletions. In some embodiments, a polypeptide variant encompasses covalent variants and derivatives. The term “derivative” is used synonymously with the term “variant” but generally refers to a molecule that has been modified and/or changed in any way relative to a reference molecule or starting molecule.

In some embodiments, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support.

In some embodiments, the polypeptide variants comprise at least one amino acid residue in a native or starting sequence removed and a different amino acid inserted in its place at the same position. Substitutions may be single, where only one amino acid in the molecule has been substituted, or they may be multiple, where two or more amino acids have been substituted in the same molecule. In some embodiments, the antigen is a polypeptide that includes 2, 3, 4, 5, 6, 7, 8, 9, 10, or more substitutions compared to a reference protein.

In some embodiments, the substitution is a conservative amino acids substitution. The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In some embodiments, protein fragments, functional protein domains, and homologous proteins are used as antigens in accordance with the present disclosure. For example, an antigen may comprise any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids which are 40%, 50%, 60%, 70%, 80%, 90%, 95%, or 100% identical to a reference protein (e.g., a protein from a microbial pathogen) herein can be utilized in accordance with the disclosure.

In some embodiments, the antigen comprises more than one (e.g., 2, 3, 4, 5, 6, 7, 8, 9, 10, or more) immunogenic proteins or polypeptides. In some embodiments, the more than one immunogenic proteins or polypeptides are derived from one protein (e.g., different fragments or one protein). In some embodiments, the more than one (e.g., from 2, 3, 4, 5, 6, 7, 8, 9, 10, or more proteins) immunogenic proteins or polypeptides are derived from multiple proteins.

In some embodiments, the antigen comprises a nucleic acid encoding an immunogenic protein or polypeptide. In some embodiments, the antigen comprises an immunogenic protein or polypeptide and a nucleic acid encoding the immunogenic protein or polypeptide. The term “nucleic acid” or “polynucleotide,” in its broadest sense, includes any compound and/or substance that comprises a polymer of nucleotides. Nucleic acids encoding immunogenic proteins or polypeptides typically comprise an open reading frame (ORF), and one or more regulatory sequences. Nucleic acids (also referred to as polynucleotides) may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, the nucleic acid encoding the immunogenic polypeptide is DNA (e.g., an expression vector for an immunogenic protein or polypeptide). In some embodiments, the nucleic acid encoding the immunogenic polypeptide is a RNA (e.g., a messenger RNA). A “messenger RNA” (mRNA) refers to any polynucleotide that encodes a (at least one) polypeptide (a naturally occurring, non-naturally occurring, or modified polymer of amino acids) and can be translated to produce the encoded polypeptide in vitro, in vivo, in situ, or ex vivo. The basic components of an mRNA molecule typically include at least one coding region, a 5′ untranslated region (UTR), a 3′ UTR, a 5′ cap and a poly-A tail.

In some embodiments, the coding region of the nucleic acid (e.g., DNA or RNA) encoding an immunogenic polypeptide is codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally occurring or wild-type sequence (e.g., a naturally occurring or wild-type mRNA sequence encoding an immunogenic protein or polypeptide). In some embodiments, a codon optimized sequence shares less than 90% sequence identity to a naturally occurring or wild-type sequence (e.g., a naturally occurring or wild-type mRNA sequence encoding an immunogenic protein or polypeptide). In some embodiments, a codon optimized sequence shares less than 85% sequence identity to a naturally occurring or wild-type sequence (e.g., a naturally occurring or wild-type mRNA sequence encoding an immunogenic protein or polypeptide). In some embodiments, a codon optimized sequence shares less than 80% sequence identity to a naturally occurring or wild-type sequence (e.g., a naturally occurring or wild-type mRNA sequence encoding an immunogenic protein or polypeptide). In some embodiments, a codon optimized sequence shares less than 75% sequence identity to a naturally occurring or wild-type sequence (e.g., a naturally occurring or wild-type mRNA sequence encoding an immunogenic protein or polypeptide).

In some embodiments, the nucleic acid encoding an immunogenic protein or polypeptide comprises one or more chemical modifications. The terms “chemical modification” and “chemically modified” refer to modification with respect to adenosine (Δ), guanosine (G), uridine (U), thymidine (T) or cytidine (C) ribonucleosides or deoxyribonucleosides in at least one of their position, pattern, percent or population.

In some embodiments, the antigen of the present disclosure is from a microbial pathogen, e.g., from a bacterium, a virus, a parasite, or a fungus. For example, the antigen may comprise a protein or polypeptide, or a nucleic acid encoding the protein or polypeptide from the microbial pathogen. In some embodiments, the antigen may comprise a microbial pathogen (e.g., a bacterial cell, a viral particle, or a fungus cell). In some embodiments, the microbial pathogen cell is live or killed. In some embodiments, the microbial pathogen is attenuated its pathogenicity. An attenuated microbial pathogen may elicit immune response but does not cause the disease that a wild-type microbial pathogen would cause.

Examples of non-limiting bacterial taxa, species, and strains, suitable for use in some embodiments of this disclosure include: Escherichia spp., Enterobacter spp. (e.g., Enterobacter cloacae), Salmonella spp. (e.g., Salmonella enteritidis, Salmonella typhi), Shigella spp., Pseudomonas spp. (e.g., Pseudomonas aeruginosa, Pseudomonas pachastrellae, Pseudomonas stutzeri), Moraxella spp. (e.g., Moraxella catarrhalis), Neisseria spp. (e.g., Neisseria gonorrhoeae, Neisseria meningitidis), Helicobacter spp., (e.g., Helicobacter pylori) Stenotrophomonas spp., Vibrio spp. (e.g., Vibrio cholerae), Legionella spp. (Legionella pneumophila), Hemophilus spp. (e.g., Hemophilus influenzae), Klebsiella spp. (e.g., Klebsiella pneumoniae), Proteus spp. (e.g., Proteus mirabilis), Serratia spp. (Serratia marcescens), Streptococcus spp., Staphylococcus spp., Corynebacterium spp., Listeria spp., and Clostridium spp., Bacillus spp. (e.g., Bacillus anthracis), Bordetella spp. (e.g., Bordetella pertussis); Borrelia spp. (e.g., Borrelia burgdorferi); Brucella spp. (e.g., Brucella abortus, Brucella canis, Brucella melitensis, Brucella suis); Campylobacter spp. (e.g., Campylobacter jejuni); Chlamydia spp. and Chlamydophila spp. (e.g., Chlamydia pneumoniae, Chlamydia trachomatis, Chlamydophila psittaci); Clostridium spp. (e.g., Clostridium botulinum, Clostridium difficile, Clostridium perfringens, Clostridium tetani); Corynebacterium spp. (e.g., Corynebacterium diphtheriae); Enterococcus spp. (e.g., Enterococcus faecalis, Enterococcus faecium); Escherichia spp. (e.g., Escherichia coli, Enterotoxic E. coli, enteropathogenic E. coli; E. coli O157:H7); Francisella spp. (e.g., Francisella tularensis); Haemophilus spp. (e.g., Haemophilus influenzae); Helicobacter spp. (e.g., Helicobacter pylori); Legionella spp. (e.g., Legionella pneumophila); Leptospira spp. (e.g., Leptospira interrogans); Listeria spp. (e.g., Listeria monocytogenes); Mycobacterium spp. (e.g., Mycobacterium leprae, Mycobacterium tuberculosis, Mycobacterium ulcerans); Mycoplasma spp. (e.g., Mycoplasma pneumoniae); Neisseria spp. (e.g., Neisseria gonorrhoeae, Neisseria meningitidis); Pseudomonas spp. (e.g., Pseudomonas aeruginosa); Rickettsia spp. (e.g., Rickettsia rickettsii); Salmonella spp. (e.g., Salmonella typhi, Salmonella typhimurium); Shigella spp. (e.g., Shigella sonnei); Staphylococcus spp. (e.g., Staphylococcus aureus, Staphylococcus epidermidis, Staphylococcus saprophyticus); Streptococcus spp. (e.g., Streptococcus agalactiae, Streptococcus pneumoniae, Streptococcus pyogenes); Treponema spp. (e.g., Treponema pallidum); Pseudodiomarina spp. (e.g., P. maritima); Marinobacter spp. (e.g., Marinobacter hydrocarbonoclasticus, Marinobacter vinifirmus) Alcanivorax spp. (e.g., Alcanivorax dieselolei); Acetinobacter spp. (e.g., A. venetianus); Halomonas spp. (e.g., H. shengliensis); Labrenzia spp.; Microbulifer spp. (e.g., M. schleiferi); Shewanella spp. (e.g., S. algae); Vibrio spp. (e.g., Vibrio cholerae, Vibrio alginolyticus, Vibrio hepatarius); and Yersinia spp. (e.g., Yersinia pestis).

In some embodiments, the bacterium is Bacillus anthracis (causing anthrax), Bordetella pertussis (causing whooping cough), Corynebacterium diphtheriae (causing diphtheria), Clostridium tetani (causing tetanus), Haemophilus influenzae type b, pneumococcus (causing pneumococcal infections), Streptococcus spp., Staphylococci spp. (including Group A or B streptococci), Mycobacterium spp. (e.g., Mycobacterium tuberculosis), Neiserria spp. (e.g., Neiserria meningitidis—causing meningococcal disease), Salmonella typhi (causing typhoid), Vibrio cholerae (causing Cholera), or Yersinia pestis (causing plague).

In some embodiments, the antigen is derived from a Gram-negative bacterium. In some embodiments, the antigen comprises a lipopolysaccharide endotoxin (LPS) from a Gram-negative bacterium. A “lipopolysaccharide endotoxin (LPS)” refers to a large molecule consisting of a lipid and a polysaccharide composed of O-antigen, outer core and inner core joined by a covalent bond. LPS is found in the outer membrane of Gram-negative bacteria. Non-limiting examples of gram-negative bacterial species include: Neisseria species including Neisseria gonorrhoeae and Neisseria meningitidis, Branhamella species including Branhamella catarrhalis, Escherichia species including Escherichia coli, Enterobacter species, Proteus species including Proteus mirabilis, Pseudomonas species including Pseudomonas aeruginosa, Pseudomonas mallei, and Pseudomonas pseudomallei, Klebsiella species including Klebsiella pneumoniae, Salmonella species, Shigella species, Serratia species, Acinetobacter species; Haemophilus species including Haemophilus influenzae and Haemophilus ducreyi; Brucella species, Yersinia species including Yersinia pestis and Yersinia enterocolitica, Francisella species including Francisella tularensis, Pasteurella species including Pasteurella multocida, Vibrio cholerae, Flavobacterium species, meningosepticum, Campylobacter species including Campylobacter jejuni, Bacteroides species (oral, pharyngeal) including Bacteroides fragilis, Fusobacterium species including Fusobacterium nucleatum, Calymmatobacterium granulomatis, Streptobacillus species including Streptobacillus moniliformis, Legionella species including Legionella pneumophila.

In some embodiments, the antigen is derived from a Gram-positive bacterium. Examples of Gram-positive bacteria include, but are not limited to, Staphylococcus spp., Streptococcus spp., Micrococcus spp., Peptococcus spp., Peptostreptococcus spp., Enterococcus spp., Bacillus spp., Clostridium spp., Lactobacillus spp., Listeria spp., Erysipelothrix spp., Propionibacterium spp., Eubacterium spp., Corynebacterium spp., Capnocytophaga spp., Bifidobacterium spp., and Gardnerella spp. In some embodiments, the Gram-positive bacterium is a bacterium of the phylum Firmicutes. In some embodiments, the Gram-positive bacterium is Streptococcus.

Other types of bacteria include acid-fast bacilli, spirochetes, and actinomycetes. Examples of acid-fast bacilli include Mycobacterium species including Mycobacterium tuberculosis and Mycobacterium leprae. Examples of spirochetes include Treponema species including Treponema pallidum, Treponema pertenue, Borrelia species including Borrelia burgdorferi (Lyme disease), and Borrelia recurrentis, and Leptospira species. Examples of actinomycetes include: Actinomyces species including Actinomyces israelii, and Nocardia species including Nocardia asteroides.

Examples of viruses include but are not limited to: Retroviruses, human immunodeficiency viruses including HIV-1, HDTV-III, LAVE, HTLV-III/LAV, HIV-III, HIV-LP, Cytomegaloviruses (CMV), Picornaviruses, polio viruses, hepatitis A virus, enteroviruses, human Coxsackie viruses, rhinoviruses, echoviruses, Calciviruses, Togaviruses, equine encephalitis viruses, rubella viruses, Flaviruses, dengue viruses, encephalitis viruses, yellow fever viruses, Coronaviruses, Rhabdoviruses, vesicular stomatitis viruses, rabies viruses, Filoviruses, ebola virus, Paramyxoviruses, parainfluenza viruses, mumps virus, measles virus, respiratory syncytial virus (RSV), Orthomyxoviruses, influenza viruses, Bungaviruses, Hantaan viruses, phleboviruses and Nairo viruses, Arena viruses, hemorrhagic fever viruses, reoviruses, orbiviruses, rotaviruses, Birnaviruses, Hepadnaviruses, Hepatitis B virus, parvoviruses, Papovaviridae, papilloma viruses, polyoma viruses, Adenoviruses, Herpesviruses including herpes simplex virus 1 and 2, varicella zoster virus, Poxviruses, variola viruses, vaccinia viruses, Irido viruses, African swine fever virus, delta hepatitis virus, non-A, non-B hepatitis virus, Hepatitis C, Norwalk viruses, astroviruses, and unclassified viruses. In some embodiments, the virus is adenovirus, enterovirus such as poliomyelitis (polio), Ebola virus, herpes viruses such as herpes simplex virus, cytomegalovirus and varicella-zoster (chickenpox and shingles), measles, mumps, rubella, hepatitis-A, -B, or -C, human papilloma virus, Influenza virus, rabies, Japanese encephalitis, rotavirus, human immunodeficiency virus (HIV), respiratory syncytial virus (RSV), smallpox, yellow fever, or Zika Virus.

In some embodiments, the antigen comprises a viral protein and/or a nucleic acid encoding a viral protein (e.g., a viral structural or non-structural protein). In some embodiments, the antigen comprises a nucleic acid encoding the viral genome. In some embodiments, the viral genome is modified to produce a modified virus that is attenuated. In some embodiments, the antigen comprises a live attenuated, inactivated, or killed virus.

In some embodiments, the antigen comprises a protein and/or a nucleic acid encoding a viral protein from a Beta Coronavirus, such as MERS-CoV, SARS-CoV-1, or SARS-CoV-2. In some embodiments, the antigen comprises a spike protein, envelope protein, membrane protein, or nucleocapsid protein from a Beta Coronavirus, and/or a nucleic acid encoding a spike protein, envelope protein, membrane protein, or nucleocapsid protein from a Beta Coronavirus. In some embodiments, the antigen comprises an immunogenic fragment of a protein from a Beta Coronavirus, and/or a nucleic acid encoding an immunogenic fragment of a protein from a Beta Coronavirus (e.g., a receptor binding domain (RBD) of Beta coronavirus spike protein). In some embodiments, the antigen comprises a live attenuated, inactivated, or killed MERS-CoV, SARS-CoV-1, or SARS-CoV-2 virus.

Examples of fungi include, but are not limited to: Cryptococcus species including Crytococcus neoformans, Histoplasma species including Histoplasma capsulatum, Coccidioides species including Coccidiodes immitis, Paracoccidioides species including Paracoccidioides brasiliensis, Blastomyces species including Blastomyces dermatitidis, Chlamydia species including Chlamydia trachomatis, Candida species including Candida albicans, Sporothrix species including Sporothrix schenckii, Aspergillus species, and fungi of mucormycosis. In some embodiments, the fungus is Candida spp., Aspergillus spp., Cryptococcus spp., Mucormycete, Blastomyces dermatitidis (causing blastomycosis), or endemic mycosis causing fungus such as Histoplasma capsulatum (causing histoplasmosis), or Sporothrix schenckii (causing sporotrichosis).

Other infectious organisms include, without limitation: parasites. Parasites include Plasmodium species, such as Plasmodium species including Plasmodium falciparum, Plasmodium malariae, Plasmodium ovale, and Plasmodium vivax and Toxoplasma gondii. Blood-borne and/or tissues parasites include Plasmodium species, Babesia species including Babesia microti and Babesia divergens, Leishmania species including Leishmania tropica, Leishmania braziliensis, Leishmania donovani, Trypanosoma species including Trypanosoma gambiense, Trypanosoma rhodesiense (African sleeping sickness), and Trypanosoma cruzi (Chagas' disease). In some embodiments, the parasite is Plasmodium spp., Leishmania, or a helminth.

Other medically relevant microorganisms have been described extensively in the literature, e.g., see C. G. A Thomas, Medical Microbiology, Bailliere Tindall, Great Britain 1983, incorporated herein by reference.

In some embodiments, the antigen is an antigen designed to provide broad heterologous protection against a range of pathogens. Heterologous immunity refers to the phenomenon whereby a history of an immune response against a stimulus or pathogen can provide a level of immunity to a second unrelated stimulus or pathogen (e.g., as described in Chen et al., Virology 2015 482: 89-97, incorporated herein by reference). For example, an antigen that induces cross-reactive memory CD8+ T cells against multiple unrelated viruses such as influenza A and Epstein-Barr Virus (EBV), as described in Watkin et al., J Allerg Clin Immunol 2017 October; 140(4) 1206-1210, incorporated herein by reference. In some embodiments, eccDNA or a functionally equivalent circular DNA induces and/or enhances heterologous protection.

In some embodiments, the antigen of the present disclosure comprises a cancer-specific antigen and/or a nucleic acid encoding such. A “cancer-specific antigen” refers to a protein that is specifically expressed or upregulated in a cancer cell, as compared to non-cancerous cells of the same origin. A cancer-specific antigen, or epitopes derived therefrom, can be recognized by the immune system to induce an immune response against the cancer. Classes of proteins that may be cancer-specific antigen include, without limitation: enzymes, receptors, and transcription factors. A large number of proteins that specifically express in cancer cells or are upregulated in cancer cells have been identified (Hassane et al., Holland-Frei Cancer Medicine. 6th edition, incorporated herein by reference). The known tumor specific antigens are classified into different classes: cancer-testis antigens (e.g., MAGE family members or NY-ESO-1), differentiation antigens (e.g., tyrosinase and Melan-A/MART-1 for melanoma, and PSA for prostate cancer), overexpressed cancer-specific antigens (e.g., Her-2/neu, Survivin, Telomerase and WT1), cancer-specific antigens arising from mutations of normal genes (e.g., mutated (3-catenin or CDK4), cancer-specific antigens arising from abnormal post-translational modifications (e.g., altered glycosylation patterns) that lead to novel epitopes in tumors (e.g., MUC1), and oncoviral proteins (e.g., human papilloma type 16 virus proteins, E6 and E7). In some embodiments, the tumor-specific antigen is expressed in a broad range of different types of cancers. In some embodiments, the tumor-specific antigen is expressed only in one or a few types of cancers.

In some embodiments, an immunostimulatory composition comprising eccDNA or a functionally equivalent circular DNA and an antigen is a vaccine composition. A “vaccine composition” is a composition that activates or enhances an individual's immune response to an antigen after the vaccine is administered to the individual. In some embodiments, a vaccine stimulates the individual's immune system to recognize the antigen as foreign and enhances the individual's immune response if the individual is later exposed to a particular pathogen, whether attenuated, inactivated, killed, or not. Vaccines may be prophylactic, for example, preventing or ameliorating a detrimental effect of a future exposure to a pathogen, or therapeutic, for example, activating the individual's immune response to a pathogen after the individual has been exposed to the pathogen. In some embodiments, a vaccine composition is used to protect or treat an organism against a disease (e.g., an infectious disease or cancer). In some embodiments, the vaccine is a subunit vaccine (e.g., a recombinant subunit vaccine), an attenuated vaccine (e.g., containing an attenuated pathogen such as a bacterial cell or a viral genome), a live vaccine (e.g., containing a live attenuated pathogen such as a bacterium or virus), or a conjugated vaccine (e.g., a vaccine containing an antigen that is not very immunogenic covalently attached to an antigen that is more immunogenic). One non-limiting example of a conjugated vaccine comprises a LPS attached to a strong protein antigen. The terms “vaccine composition” and “vaccine” are used interchangeably herein.

Vaccines that contain cancer-specific antigens are termed herein as “cancer vaccine.” Cancer vaccines induce cancer-specific immune response against a cancer or a cancer-specific antigen. Such an immune response is effective in inhibiting cancer growth and/or preventing reoccurrence of tumor. Cancer vaccines may be used for cancer immunotherapy, which is a type of cancer treatment designed to boost the body's natural defenses to fight the cancer. It uses substances either made by the body or in a laboratory to improve or restore immune system function.

In some embodiments, a vaccine composition comprises eccDNA or a functionally equivalent circular DNA, an antigen, and one or more adjuvants. An “adjuvant” refers to a pharmacological or immunological agent that modifies the effect of other agents, for example, of an antigen in a vaccine. Adjuvants are typically included in vaccines to enhance the recipient individual's immune response to an antigen. The use of adjuvants allows the induction of a greater immune response in an individual with the same dose of antigen, or the induction of a similar level of immune response with a lower dose of injected antigen. Adjuvants are thought to function in several ways, including by increasing the surface area of antigen, prolonging the retention of the antigen in the body thus allowing time for the lymphoid system to have access to the antigen, slowing the release of antigen, targeting antigen to macrophages, activating macrophages, activating leukocytes such as antigen-presenting cells (e.g., monocytes, macrophages, and/or dendritic cells), or otherwise eliciting broad activation of the cells of the immune system see, e.g., H. S. Warren et al, Annu. Rev. immunol., 4:369 (1986), incorporated herein by reference. The ability of an adjuvant to induce and increase a specific type of immune response and the identification of that ability is thus a key factor in the selection of particular adjuvants for vaccine use against a particular pathogen. Adjuvants that are known to those of skill in the art, include, without limitation: aluminum salts (referred to herein as “alum”), liposomes, lipopolysaccharide (LPS) or derivatives such as monophosphoryl lipid A (MPLA) and glycopyranosyl lipid A (GLA), molecular cages for antigen, components of bacterial cell walls, endocytosed nucleic acids such as double stranded RNA (dsRNA), single-stranded DNA (ssDNA), and unmethylated CpG dinucleotide-containing DNA. Typical adjuvants include water and oil emulsions, e.g., Freund's adjuvant and MF59, and chemical compounds such as aluminum hydroxide or alum. At present, currently licensed vaccines in the United States contain only a limited number of adjuvants, such as alum that enhances production of subtype 2 helper T (Th2) cells and MPLA which activates innate immunity via Toll-like receptor 4 (TLR4). Many of the most effective adjuvants include bacteria or their products, e.g., microorganisms such as the attenuated strain of Mycobacterium bovis, Bacille Calmette-Gudrin (BCG); microorganism components, e.g., alum-precipitated diphtheria toxoid, bacterial lipopolysaccharides (“endotoxins”) and their derivatives such as MPLA and GLA.

In some embodiments, a vaccine composition is formulated for administration to an individual. In some embodiments, a vaccine composition is formulated or administered in combination with one or more pharmaceutically acceptable excipients. In some embodiments, vaccine compositions comprise at least one additional active substance, such as, for example, a therapeutically active substance, a prophylactically active substance, or a combination of both. Vaccine compositions may be sterile, pyrogen-free, or both. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

Formulations of vaccine compositions may be prepared by any method known or hereafter developed in the art of pharmacology. In general, such preparatory methods include the step of bringing the antigen and/or the adjuvant (e.g., eccDNA or a functionally equivalent circular DNA) into association with an excipient and/or one or more other accessory ingredients, and then, if necessary and/or desirable, dividing, shaping and/or packaging the product into a desired single- or multi-dose unit.

Relative amounts of eccDNA or a functionally equivalent circular DNA, the antigen, the pharmaceutically acceptable excipient, and/or any additional ingredients in a pharmaceutical composition (e.g., a second adjuvant) in accordance with the disclosure will vary, depending upon the identity, size, and/or condition of the individual treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, a vaccine composition is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation an encoded protein in vivo; and/or (6) alter the release profile of a protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with DNA or RNA vaccines (e.g., for transplantation into an individual), hyaluronidase, nanoparticle mimics and combinations thereof.

In some embodiments, a vaccine composition is formulated in an aqueous solution. In some embodiments, a vaccine composition is formulated in a nanoparticle. In some embodiments, a vaccine composition is formulated in a lipid nanoparticle. In some embodiments, a vaccine composition is formulated in a lipid-polycation complex, referred to as a lipid nanoparticle. The formation of the lipid nanoparticle may be accomplished by methods known in the art and/or as described in U.S. Pub. No. 20120178702, incorporated herein by reference. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is incorporated herein by reference. In some embodiments, a vaccine composition is formulated in a lipid nanoparticle that includes a non-cationic lipid such as, but not limited to, cholesterol or dioleoyl phosphatidylethanolamine (DOPE).

A lipid nanoparticle formulation may be influenced by, but not limited to, the selection of the ionizable lipid component, the degree of ionizable lipid saturation, the nature of the PEGylation, ratio of all components and biophysical parameters such as size. In one example by Semple et al. (Nature Biotech. 2010 28:172-176; incorporated herein by reference), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (Basha et al. Mol Ther. 2011 19:2186-2200; incorporated herein by reference).

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0% and/or 3.0% to 6.0% of the lipid molar ratio of PEG-c-DOMG (R-3-[(o-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, a vaccine formulation is a nanoparticle that comprises at least one lipid (termed a “lipid nanoparticle” or “LNP”). The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, incorporated herein by reference. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In some embodiments, a lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of 20-60% ionizable cationic lipid: 5-25% neutral lipid: 25-55% sterol; 0.5-15% PEG-lipid.

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

The lipid nanoparticles may be made in a sterile environment by the system and/or methods described in US Patent Publication No. US20130164400, incorporated herein by reference.

In some embodiments, the lipid nanoparticle formulation may be formulated by the methods described in International Publication Nos. WO2011127255 or WO2008103276, the contents of each of which are herein incorporated by reference in their entirety. As a non-limiting example, the eccDNA or a functionally equivalent circular DNA and/or an antigen may be encapsulated in LNP formulations as described in WO2011127255 and/or WO2008103276; the contents of each of which are herein incorporated by reference in their entirety.

In some embodiments, lipid nanoparticle formulations may comprise a polycationic composition. As a non-limiting example, the polycationic composition may be selected from formula 1-60 of US Patent Publication No. US20050222064; the content of which is incorporated herein by reference. In another embodiment, the LNP formulations comprising a polycationic composition may be used for the delivery of the modified RNA in vivo and/or in vitro.

In some embodiments, the lipid nanoparticle formulations may additionally comprise a permeability enhancer molecule. Non-limiting permeability enhancer molecules are described in US Patent Publication No. US20050222064; the content of which is incorporated herein by reference.

In some embodiments, vaccine compositions may be formulated in liposomes such as, but not limited to, DiLa2 liposomes (Marina Biotech, Bothell, WA), SMARTICLES® (Marina Biotech, Bothell, WA), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); incorporated herein by reference) and hyaluronan-coated liposomes (Quiet Therapeutics, Israel).

In some embodiments, vaccine compositions may be formulated in a lyophilized gel-phase liposomal composition as described in US Publication No. US2012060293, incorporated herein by reference.

In some embodiments, vaccine compositions may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles may have a diameter from about 10 to 500 nm. In some embodiments, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, a vaccine composition is formulated in a liposome. Liposomes are vehicles, such as artificially prepared vesicles, which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, each of which is incorporated by reference herein in its entirety.

In some embodiments, vaccine compositions may include, without limitation, liposomes such as those formed from 1,2-dioleyloxy-N,N-dimethylaminopropane (DODMA) liposomes, DiLa2 liposomes from Marina Biotech (Bothell, WA), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; incorporated herein by reference) and liposomes which may deliver small molecule drugs such as, but not limited to, DOXIL® from Janssen Biotech, Inc. (Horsham, PA).

In some embodiments, pharmaceutical compositions may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties). The original manufacture method by Wheeler et al. was a detergent dialysis method, which was later improved by Jeffs et al. and is referred to as the spontaneous vesicle formation method

In some embodiments, liposomes may be formulated for targeted delivery. As a non-limiting example, the liposome may be formulated for targeted delivery to the liver. The liposome used for targeted delivery may include, but is not limited to, the liposomes described in and methods of making liposomes described in US Patent Publication No. US20130195967, the contents of which are incorporated herein by reference.

In some embodiments, the eccDNA or a functionally equivalent circular DNA and/or antigen may be formulated in a cationic oil-in-water emulsion where the emulsion particle comprises an oil core and a cationic lipid which can interact with the polynucleotide anchoring the molecule to the emulsion particle (see International Pub. No. WO2012006380; incorporated herein by reference).

In some embodiments, the eccDNA or a functionally equivalent circular DNA and/or antigen may be formulated in a water-in-oil emulsion comprising a continuous hydrophobic phase in which the hydrophilic phase is dispersed. As a non-limiting example, the emulsion may be made by the methods described in International Publication No. WO201087791, the contents of which are incorporated herein by reference.

The eccDNA or functionally equivalent circular DNA, the antigen, and/or optionally the second adjuvant may be formulated using any of the methods described herein or known in the art separately or together. For example, the eccDNA or a functionally equivalent circular DNA and the antigen may be formulated in one lipid nanoparticle or two separately lipid nanoparticles. In some embodiments, the eccDNA or functionally equivalent circular DNA and the antigen are formulated in the same aqueous solution or two separate aqueous solutions. In some embodiments, the eccDNA or functionally equivalent circular DNA, are adsorbed onto alum (e.g., as described in Jones et al., Journal of Biological Chemistry 280, 13406-13414, 2005, incorporated herein by reference).

In some embodiments, a vaccine composition comprises two or more adjuvants (also referred to as an “adjuvant system”). The adjuvant system comprises two or more other adjuvants described herein or well known within the art.

### Formulations of Immunostimulatory Compositions

In some embodiments, an immunostimulatory composition is formulated for administration to an individual. In some embodiments, an immunostimulatory composition further comprises a pharmaceutically acceptable carrier. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” means a pharmaceutically acceptable material, composition or vehicle, such as a liquid or solid filler, diluent, excipient, solvent or encapsulating material, involved in carrying or transporting agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of an immunostimulatory composition are also capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

An immunostimulatory composition may conveniently be presented in unit dosage form and may be prepared by any of the methods well-known in the art of pharmacy. The term “unit dose” when used in reference to an immunostimulatory composition of the present disclosure refers to physically discrete units suitable as unitary dosage for the individual, each unit containing a predetermined quantity of active material calculated to produce the desired therapeutic effect in association with the required diluent, carrier, or vehicle. Relative amounts of the active ingredient, the pharmaceutically acceptable excipient, and/or any additional ingredients in a pharmaceutical composition will vary, depending upon the identity, size, and/or condition of the individual treated and further depending upon the route by which the composition is to be administered. The composition may comprise between 0.1% and 100% (w/w) active ingredient.

Pharmaceutically acceptable excipients used in the manufacture of pharmaceutical compositions include inert diluents, dispersing and/or granulating agents, surface active agents and/or emulsifiers, disintegrating agents, binding agents, preservatives, and/or buffering agents.

Examples of diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Examples of granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Examples of surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Examples of binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Examples of preservatives include antioxidants, chelating agents, antimicrobial preservatives, antifungal preservatives, antiprotozoan preservatives, alcohol preservatives, acidic preservatives, and other preservatives.

In some embodiments, a preservative is an antioxidant. Examples of antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

In some embodiments, a preservative is a chelating agent. Examples of chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Examples of antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

In some embodiments, a preservative is an antifungal agent. Examples of antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

In some embodiments, a preservative is an alcohol. Examples of alcohol preservatives include ethanol, polyethylene glycol, phenol, phenolic compounds, bisphenol, chlorobutanol, hydroxybenzoate, and phenylethyl alcohol.

In some embodiments, a preservative is an acidic preservative. Examples of acidic preservatives include vitamin A, vitamin C, vitamin E, beta-carotene, citric acid, acetic acid, dehydroacetic acid, ascorbic acid, sorbic acid, and phytic acid.

Other examples of preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Examples of buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Solid dosage forms for enteral (e.g., oral) administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent. Solid compositions may also contain one or more encapsulating agents, such as polymeric substances and waxes. Solid compositions may release the active ingredient(s) only, or preferentially, in a certain part of the intestinal tract, optionally, in a delayed manner. Solid compositions may the active ingredient in a micro-encapsulated form with one or more excipients as noted above.

Liquid dosage forms for enteral (e.g., oral) and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, natural oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, the conjugates are mixed with solubilizing agents such as Cremophor®, alcohols, oils, modified oils, glycols, polysorbates, cyclodextrins, polymers, and mixtures thereof.

For topical administration, an immunostimulatory composition can be formulated into ointments, salves, gels, or creams, as is generally known in the art. Topical administration can utilize transdermal delivery systems well known in the art. An example is a dermal patch.

The formulation of an immunostimulatory composition may dependent upon the route of administration. Injectable preparations suitable for parenteral administration (e.g., intravenous, intramuscular, subcutaneous, intradermal, intratumoral, peritumoral, intralesional, or perilesional administration) include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In some embodiments, an immunostimulatory composition used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. An immunostimulatory composition will ordinarily be stored in lyophilized form or as an aqueous solution if it is highly stable to thermal and oxidative denaturation. The pH of the preparations typically will be about from 6 to 8, although higher or lower pH values can also be appropriate in certain instances.

In some embodiments, the present disclosure contemplates an immunostimulatory composition comprising a pharmaceutically acceptable injectable vehicle. The immunostimulatory compositions of the present disclosure may be administered in conventional vehicles with or without other standard carriers, in the form of injectable solutions or suspensions. The added carriers might be selected from agents that elevate total immune response in the course of the immunization procedure. Liposomes have been suggested as suitable carriers. The insoluble salts of aluminum, that is aluminum phosphate or aluminum hydroxide, have been utilized as carriers in routine clinical applications in humans. Polynucleotides and polyelectrolytes and water-soluble carriers such as muramyl dipeptides have been used.

Preparation of injectable compositions of the present disclosure, includes mixing the eccDNA or functionally equivalent circular DNA with muramyl dipeptides or other carriers. The resultant mixture may be emulsified in a mannide monooleate/squalene or squalane vehicle. Four parts by volume of squalene and/or squalane are used per part by volume of mannide monooleate. Methods of formulating injectable compositions are well-known to those of ordinary skill in the art. (Rola, Immunizing Agents and Diagnostic Skin Antigens. In: Remington's Pharmaceutical Sciences, 18th Edition, Gennaro (ed.), (Mack Publishing Company 1990) pages 1389-1404).

Additional pharmaceutical carriers may be employed to control the duration of action of an immunostimulatory composition in a therapeutic application. Control release preparations can be prepared through the use of polymers to complex or adsorb chimeric construct. For example, biocompatible polymers include matrices of poly(ethylene-co-vinyl acetate) and matrices of a polyanhydride copolymer of a stearic acid dimer and sebacic acid. (Sherwood et al. (1992) Bio/Technology 10: 1446). The rate of release of the chimeric construct from such a matrix depends upon the molecular weight of the construct, the amount of the construct within the matrix, and the size of dispersed particles. (Saltzman et al. (1989) Biophys. J. 55: 163; Sherwood et al, supra.; Ansel et al. Pharmaceutical Dosage Forms and Drug Delivery Systems, 5th Edition (Lea & Febiger 1990); and Gennaro (ed.), Remington's Pharmaceutical Sciences, 18th Edition (Mack Publishing Company 1990)). The chimeric construct can also be conjugated to polyethylene glycol (PEG) to improve stability and extend bioavailability times (e.g., Katre et al.; U.S. Pat. No. 4,766,106).

In some embodiments, an immunostimulatory composition that is formulated for administration to an individual is a vaccine composition. Unless otherwise noted, pharmaceutically acceptable carriers that are suitable for the formulation of immunostimulatory compositions are also suitable for the formulation of vaccine compositions. As with immunostimulatory compositions, the formulation of a vaccine composition may depend on the route of administration. A vaccine composition may be formulated, for example, for enteral (e.g., oral), parenteral (e.g., intravenous, intramuscular, subcutaneous, intradermal, intratumoral, peritumoral, intralesional, or perilesional administration), or topical administration to an individual.

### Methods for Eliciting an Immune Response

Other aspects of the present disclosure provide methods of eliciting or enhancing an immune response in an individual. In some embodiments, the methods comprise administering to the individual an effective (e.g., therapeutically effective) amount of eccDNA or a functionally equivalent circular DNA (e.g., for enhancing an innate immune response, including induction of heterologous or “trained” immunity or innate memory). In some embodiments, the methods further comprise administering to the individual an effective (e.g., therapeutically effective) amount of an antigen. In some embodiments, eccDNA or a functionally equivalent circular DNA is administered separately from the antigen. In some embodiments, eccDNA or a functionally equivalent circular DNA is administered prior to administering the antigen. In some embodiments, eccDNA or a functionally equivalent circular DNA is administered after administering the antigen. In some embodiments, eccDNA or a functionally equivalent circular DNA and the antigen are administered simultaneously. In some embodiments, eccDNA or a functionally equivalent circular DNA and the antigen are administered as an admixture.

The eccDNA or a functionally equivalent circular DNA and/or the antigen (e.g., the eccDNA or a functionally equivalent circular DNA alone, the antigen alone, or the eccDNA or a functionally equivalent circular DNA and the antigen together) elicits an immune response in the individual. In some embodiments, the eccDNA or a functionally equivalent circular DNA and/or the antigen activates cytokine and/or chemokine (e.g., IFNα, IFNβ, IFNγ, IL-6, TNFα) production. In some embodiments, the immune response is an innate immune response. In some embodiments, the immune response is an adaptive immune response specific to an antigen in an immunostimulatory composition. In some embodiments, the eccDNA or a functionally equivalent circular DNA and/or the antigen activates B cell immunity. In some embodiments, the eccDNA or a functionally equivalent circular DNA and/or the antigen elicits antibody production. In some embodiments, the eccDNA or a functionally equivalent circular DNA and/or the antigen activates cytotoxic T cells specific to the antigen.

In some embodiments, the eccDNA or a functionally equivalent circular DNA, whether administered alone or with an antigen, enhances the innate immune response, compared to the innate immune response that occurs in the absence of eccDNA or a functionally equivalent circular DNA, or when the antigen is administered alone. In some embodiments, the eccDNA or a functionally equivalent circular DNA activates peripheral blood mononuclear cells (PBMCs). In some embodiments, the number of PBMCs that are activated is increased by at least 20% in the presence of the eccDNA or a functionally equivalent circular DNA, compared to the absence of eccDNA or a functionally equivalent circular DNA, or when the antigen is administered alone. For example, the number of PBMCs that are activated may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more. In some embodiments, the number of PBMCs that are activated is increased by 20%, 30%, 40%, 50%, 60%, 70%, 80%, 90%, 100%, 2-fold, 5-fold, 10-fold, 100-fold, 1000-fold or more.

In some embodiments, the eccDNA or a functionally equivalent circular DNA activates a pattern recognition receptor (PRR). In some embodiments, the PRR is STING. In some embodiments, the PPR activation is increased by at least 20% in the presence the eccDNA or a functionally equivalent circular DNA, compared to the absence of eccDNA or a functionally equivalent circular DNA, or when the antigen is administered alone. For example, PPR activation may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more. In some embodiments, the number of PRRs that are activated is increased by 20%, 30%, 40%, 50%, 60%, 70%, 80%, 90%, 100%, 2-fold, 5-fold, 10-fold, 100-fold, 1000-fold or more.

In some embodiments, the eccDNA or a functionally equivalent circular DNA induces the production of proinflammatory cytokines and/or chemokines in the individual (e.g., IFNα, IFNβ, IFNγ, IL-6, TNFα). In some embodiments, the level of proinflammatory cytokines and/or chemokines is increased by at least 20% in the presence of the eccDNA or a functionally equivalent circular DNA, compared to the absence of eccDNA or a functionally equivalent circular DNA, or when the antigen is administered alone. For example, the level of proinflammatory cytokines and/or chemokines may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more. In some embodiments, the level of proinflammatory cytokines and/or chemokines is increased by 20%, 30%, 40%, 50%, 60%, 70%, 80%, 90%, 100%, 2-fold, 5-fold, 10-fold, 100-fold, 1000-fold or more.

In some embodiments, the eccDNA or a functionally equivalent circular DNA enhances innate immune memory (also referred to as trained immunity). “Innate immune memory” confers heterologous immunity that provides broad protection against a range of pathogens. In some embodiments, the innate immune memory is increased by at least 20% in the presence of the eccDNA or a functionally equivalent circular DNA, compared to the absence of eccDNA or a functionally equivalent circular DNA, or when the antigen is administered alone. For example, the innate immune memory may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more. In some embodiments, the innate immune memory is increased by 20%, 30%, 40%, 50%, 60%, 70%, 80%, 90%, 100%, 2-fold, 5-fold, 10-fold, 100-fold, 1000-fold or more.

In some embodiments, the eccDNA or a functionally equivalent circular DNA, when administered as an admixture with an antigen (e.g., as a vaccine composition), enhances the anti-specific immune response against the antigen or against the invading agent from which the antigen is derived (e.g., a microbial pathogen or cancer), compared to in the absence of eccDNA or a functionally equivalent circular DNA, e.g., when the antigen is administered alone. In some embodiments, the eccDNA or a functionally equivalent circular DNA enhances the production of antigen-specific antibodies (i.e., immunoglobulins) in the individual (e.g., by at least 20%), compared to the absence of eccDNA or a functionally equivalent circular DNA, e.g., when the antigen is administered alone. For example, the eccDNA or a functionally equivalent circular DNA may enhance the production of antigen-specific antibodies (i.e. immunoglobulins) by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more in the individual, compared to in the absence of eccDNA or a functionally equivalent circular DNA, e.g., when the antigen is administered alone. In some embodiments, the eccDNA or a functionally equivalent circular DNA enhances the production of antigen-specific antibodies (i.e., immunoglobulins) by 20%, 30%, 40%, 50%, 60%, 70%, 80%, 90%, 100%, 2-fold, 5-fold, 10-fold, 100-fold, 1000-fold or more, compared to the absence of eccDNA or a functionally equivalent circular DNA, e.g., when the antigen is administered alone. Antigen-specific antibodies may be of any isotype, such as immunoglobulin A (IgA), immunoglobulin D (IgD), immunoglobulin E (IgE), immunoglobulin G (IgG), or immunoglobulin M (IgM). Antigen-specific antibodies that are IgGs may be those of any subclass, such as IgG1, IgG2, IgG3, or IgG4. One skilled in the art is familiar with how to evaluate the level of an antibody titer, e.g., by enzyme-linked immunosorbent assay (ELISA).

In some embodiments, the eccDNA or a functionally equivalent circular DNA enhances the activation of cytotoxic T-cells (e.g., by at least 20%) in the individual, compared to the absence of eccDNA or a functionally equivalent circular DNA, e.g., when the antigen is administered alone. For example, the eccDNA or a functionally equivalent circular DNA may enhance activation of cytotoxic T-cells by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the individual, compared to the absence of eccDNA or a functionally equivalent circular DNA, e.g., when the antigen is administered alone. In some embodiments, the eccDNA or a functionally equivalent circular DNA enhances the activation of cytotoxic T-cells by 20%, 30%, 40%, 50%, 60%, 70%, 80%, 90%, 100%, 2-fold, 5-fold, 10-fold, 100-fold, 1000-fold or more, compared to the absence of eccDNA or a functionally equivalent circular DNA, e.g., when the antigen is administered alone.

It has been demonstrated that the innate immune system plays a crucial role in the control of initiation of the adaptive immune response and in the induction of appropriate cell effector responses. (Fearon et al. (1996) Science 272: 50-3; Medzhitov et al. (1997) Cell 91: 295-8, incorporated herein by reference). As such, in some embodiments, the eccDNA or a functionally equivalent circular DNA enhances the innate immune response in an individual (e.g., when administered alone or in an admixture with an antigen), which in turn enhances the adaptive immune response against the antigen in the individual. This is particular useful in individuals that have undeveloped (e.g., in a neonatal infant), weak (e.g., in an elderly), or compromised immune systems (e.g., in a patient with primary immunodeficiency or acquired immunodeficiency secondary to HIV patient infection or a cancer patient undergoing with or without chemotherapy and/or radiation therapy).

In some embodiments, the eccDNA or a functionally equivalent circular DNA prolongs the effect of a vaccine (e.g., by at least 20%) in the individual, compared to without eccDNA or a functionally equivalent circular DNA (e.g., when the antigen is administered alone). For example, the eccDNA or a functionally equivalent circular DNA may prolong the effect of a vaccine by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the individual, compared to the absence of eccDNA or a functionally equivalent circular DNA, e.g., when the antigen is administered alone. In some embodiments, the eccDNA or a functionally equivalent circular DNA prolongs the effect of a vaccine by 20%, 30%, 40%, 50%, 60%, 70%, 80%, 90%, 100%, 2-fold, 5-fold, 10-fold, 100-fold, 1000-fold or more, compared to the absence of eccDNA or a functionally equivalent circular DNA, e.g., when the antigen is administered alone.

In some embodiments, the eccDNA or a functionally equivalent circular DNA increases rate of (accelerates) an immune response, compared to the absence of eccDNA or a functionally equivalent circular DNA, e.g., when the antigen is administered alone. For example, the eccDNA or a functionally equivalent circular DNA may increase the rate of an immune response by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more. In some embodiments, the eccDNA or a functionally equivalent circular DNA increases the rate of an immune response by 20%, 30%, 40%, 50%, 60%, 70%, 80%, 90%, 100%, 2-fold, 5-fold, 10-fold, 100-fold, 1000-fold or more. The phrase “increase the rate of immune response” mean it takes less time for the immune system of an individual to react to an invading agent (e.g., a microbial pathogen).

In some embodiments, the antigen elicits a same level of immune response at a lower dose in the presence of the eccDNA or a functionally equivalent circular DNA, compared to in the absence of eccDNA or a functionally equivalent circular DNA, e.g., when the antigen is administered alone. In some embodiments, the amount of antigen needed to produce the same level of immune response is reduced by at least 20% in the presence of the eccDNA or a functionally equivalent circular DNA, compared to in the absence of eccDNA or a functionally equivalent circular DNA, e.g., when the antigen is administered alone. For example, the amount of antigen needed to produce the same level of immune response may be reduced by at least 20%, at least 30%, at least 40%, at least 50%, at least 60%, at least 70%, at least 80%, at least 90%, at least 95%, at least 99% or more. In some embodiments, the amount of antigen needed to produce the same level of immune response is reduced by at 20%, 30%, 40%, 50%, 60%, 70%, 80%, 90%, 95%, 99% or more.

The prophylactic or therapeutic use of the eccDNA or a functionally equivalent circular DNA, or an immunostimulatory composition comprising eccDNA or a functionally equivalent circular DNA, is also within the scope of the present disclosure. In some embodiments, the immunostimulatory composition are used in methods of vaccinating an individual by prophylactically administering to the individual an effective amount of the immunostimulatory composition (e.g., as a vaccine composition). “Vaccinating an individual” refers to a process of administering an immunogen, typically an antigen formulated into a vaccine, to the individual in an amount effective to increase or activate an immune response against the antigen and, thus, against a pathogen displaying the antigen. In some embodiments, the terms do not require the creation of complete immunity against the pathogen. In some embodiments, the terms encompass a clinically favorable enhancement of an immune response toward the antigen or pathogen. Methods for immunization, including formulation of vaccine compositions and selection of doses, routes of administration and the schedule of administration (e.g., primary dose and one or more booster doses), are well known in the art. In some embodiments, vaccinating an individual reduces the risk of developing a disease (e.g., an infectious disease or cancer) in the individual.

In some embodiments, an immunostimulatory composition comprising the eccDNA or a functionally equivalent circular DNA and optionally an antigen are used in methods of treating or preventing a disease (e.g., an infectious disease or cancer) by administering to the individual an effective amount of the immunostimulatory composition. In such embodiments, the antigen is an antigen specific to the biological agent known to cause the disease (e.g., a pathogen or a cancer cell)

In some embodiments, the disease is an infectious disease. An “infectious disease” refers to an illness caused by a pathogen that results from transmission from an infected person, animal, or reservoir to a susceptible host, either directly or indirectly, through an intermediate plant or animal host, vector, or inanimate environment. See Last J M. ed. A dictionary of epidemiology. 4th ed., New York: Oxford University Press, 1988. Infectious disease is also known as transmissible disease or communicable disease. In some embodiments, infectious diseases may be asymptomatic for much or even all of their course in a given host. Infectious pathogens include some viruses, bacteria, fungi, protozoa, multicellular parasites, and aberrant proteins known as prions. In some embodiments, the infectious disease is caused by any of the microbial pathogens (e.g., a bacterium, a virus, a parasite, or a fungus) described herein or known to one skilled in the art. In some embodiments, the infectious disease is caused by Plasmodium spp. (malaria), Bacillus anthracis (anthrax), Bordetella pertussis (whooping cough), Corynebacterium diphtheriae (diphtheria), Clostridium tetani (tetanus), Haemophilus influenzae type b, pneumococcus (pneumococcal infections), Streptococcus spp., Staphylococci spp., Group A or B streptococci, Mycobacterium spp. (e.g., Mycobacterium tuberculosis), Neiserria spp. (e.g., Neiserria meningitidis—meningococcal disease), Salmonella typhi (typhoid), Vibrio cholerae (Cholera), or Yersinia pestis (plague).

In some embodiments, the infectious disease is caused by adenovirus, enterovirus such as polio virus, dengue virus, Ebola virus, herpes viruses such as herpes simplex virus, cytomegalovirus and varicella-zoster (chickenpox and shingles), measles, mumps, rubella, hepatitis-A, -B, or -C, human papilloma virus, Influenza virus, rabies, Japanese encephalitis, rotavirus, human immunodeficiency virus (HIV), respiratory syncytial virus (RSV), smallpox, yellow fever, dengue virus, or Zika Virus. In some embodiments, the infectious disease is caused by malaria, Leishmania, or a helminth. In some embodiments, the infectious disease is caused by Candida spp., Aspergillus spp., Cryptococcus spp., Mucormycete, Blastomyces dermatitidis, Histoplasma capsulatum, or Sporothrix schenckii. In some embodiments, the infectious disease is caused by prion. In some embodiments, the infectious disease is sepsis.

In some embodiments, an immunostimulatory composition may be administered in combination with another therapeutic agent for the infectious diseases. Such other therapeutic agents may be, without limitation: antibiotics, anti-viral agents, anti-fungal agents, or anti-parasitic agents. One skilled in the art is familiar with how to select or administer the additional therapeutic agent based on the disease to be treated.

In some embodiments, the disease is cancer. Compositions comprising cancer-specific antigens and the eccDNA or a functionally equivalent circular DNA may be used in cancer immunotherapy by eliciting cancer-specific immune response against the cancer. The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Examples of cancers include, but are not limited to, hematological malignancies. Additional Examples of cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer treated using the composition and methods of the present disclosure is melanoma.

In some embodiments, the immune response of an individual is elicited or enhanced by administering eccDNA or a functionally equivalent circular DNA, optionally including an antigen, by injection. Injection of eccDNA or a functionally equivalent circular DNA, such as in the form of an immunostimulatory composition, may occur parenterally, e.g., intravenously, intramuscularly, subcutaneously, or intradermally. Techniques for administering injectable compositions to individuals are well known by those of ordinary skill in the art and may be achieved, for example, through the use of a syringe fitted with a hypodermic needle. For intradermal administration, suitable devices include short needle devices which limit the effective penetration length of a needle into the skin. Alternatively or additionally, conventional syringes can be used in the classical Mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable. Administration of immunostimulatory compositions may also occur topically or orally.

Other delivery systems can include time-release, delayed release or sustained release delivery systems. Such systems can avoid the need for repeated administrations, increasing convenience to the individual and the administering physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide-based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

### Kits

Also encompassed by the disclosure are kits (e.g., pharmaceutical packs). The kits provided may comprise an immunostimulatory composition (e.g., comprising eccDNA or a functionally equivalent circular DNA) and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound. In some embodiments, the pharmaceutical composition or compound provided in the first container and the second container are combined to form one unit dosage form.

Thus, in one aspect, provided are kits including a first container comprising a compound or pharmaceutical composition. In certain embodiments, the kits are useful for treating a disease (e.g., infectious disease, proliferative disease, inflammatory disease, autoimmune disease, and/or chronic disease) in an individual in need thereof. In certain embodiments, the kits are useful for preventing a disease (e.g., infectious disease, proliferative disease, inflammatory disease, autoimmune disease, and/or chronic disease) in an individual in need thereof. In certain embodiments, the kits are useful for enhancing or eliciting an immune response (e.g., innate and/or adaptive immune response) in an individual.

In certain embodiments, a kit further includes instructions for using the compound or pharmaceutical composition included in the kit. A kit may also include information as required by a regulatory agency such as the U.S. Food and Drug Administration (FDA). In certain embodiments, the information included in the kits is prescribing information. In certain embodiments, the kits and instructions provide for treating a disease (e.g., infectious disease, proliferative disease, inflammatory disease, autoimmune disease, and/or chronic disease) in an individual in need thereof. In certain embodiments, the kits and instructions provide for preventing a disease (e.g., infectious disease, proliferative disease, inflammatory disease, autoimmune disease, and/or chronic disease) in an individual in need thereof. In certain embodiments, the kits and instructions provide for eliciting or enhancing of an immune response (e.g., innate and/or adaptive immune response) in an individual to which contents of the kit are administered. In some embodiments, the kits and instructions provide for use of eccDNA or a functionally equivalent circular DNA in a vaccine for a disease, (e.g., infectious disease, proliferative disease, inflammatory disease, autoimmune disease, and/or chronic disease) in an individual to which contents of the kit are administered. A kit may include one or more additional pharmaceutical agents, e.g., pharmaceutical agents which are also useful for treating or preventing a disease, as a separate composition.

### EXAMPLES

### Example 1—Development of an Efficient and Robust Method for eccDNA Purification

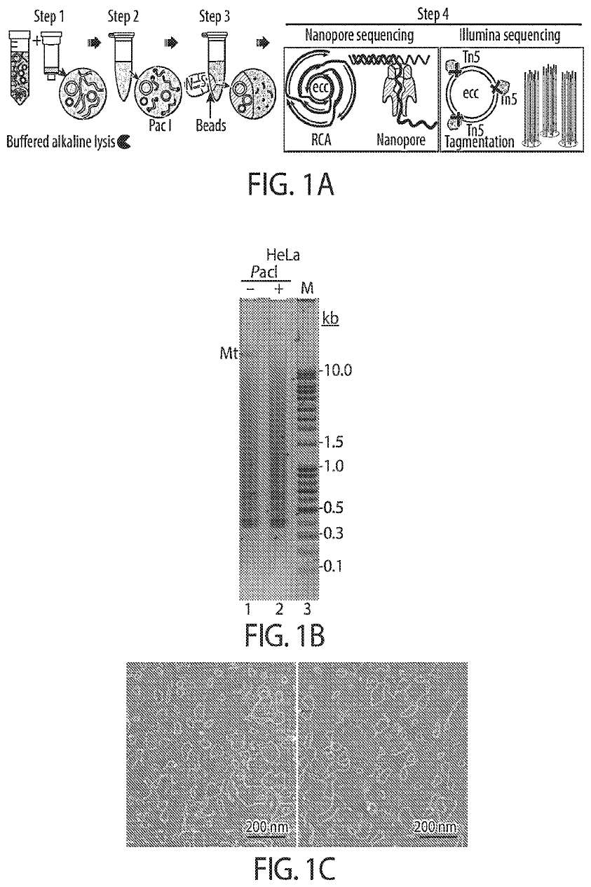

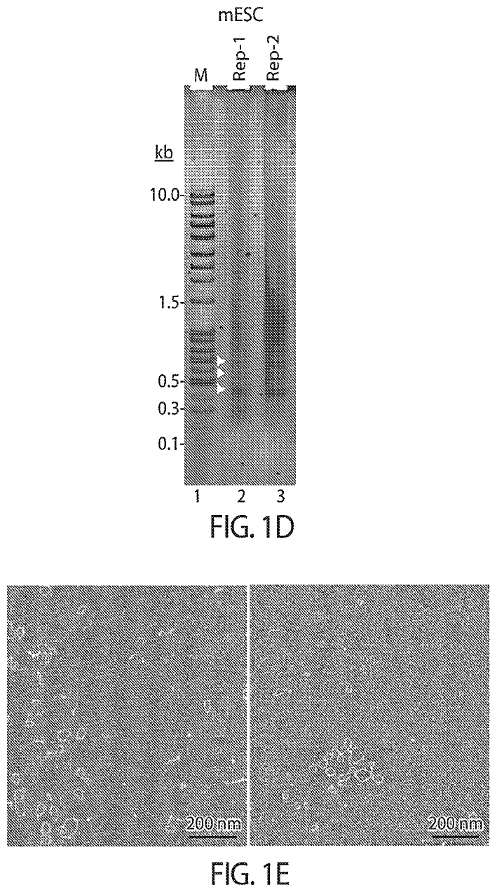

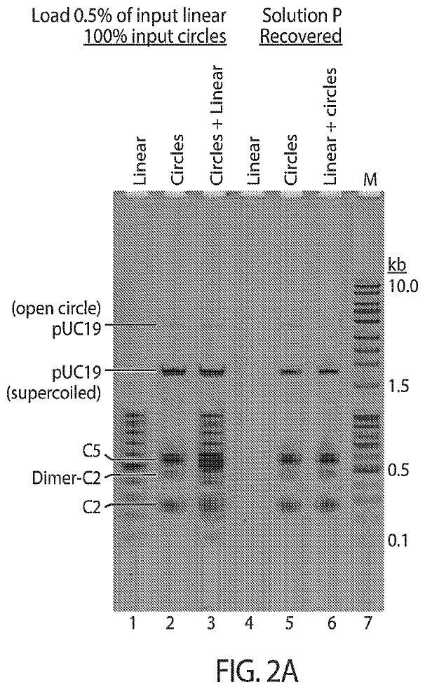

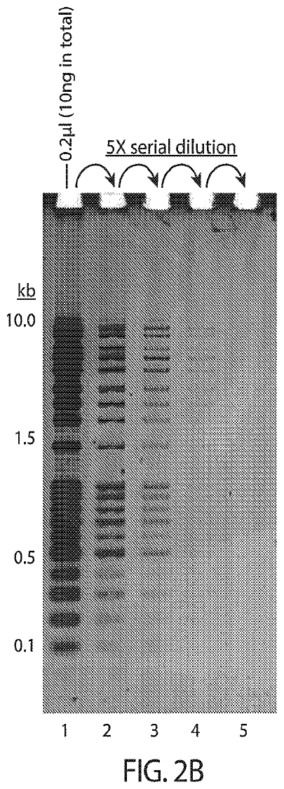

A new three-step eccDNA enrichment method was developed that allows obtaining highly purified eccDNAs (FIG. 1A). In the first step, to minimize eccDNA loss, crude eccDNAs are extracted by using modified alkaline lysis at pH 11.8, rather than by use of conventional unbuffered sodium hydroxide that could cause irreversible denaturation or breakage of circular DNA¹⁸. In the second step, to remove mitochondrial DNA (mtDNA), a rare-cutter PacI restriction enzyme is used to linearize mtDNA (both human and rodent mtDNA have 3 PacI restriction sites) before an exonuclease (ATP-dependent plasmid safe DNase) is used to digest linear DNA. In the third step, a solution (Solution A) that can selectively recover circular, but not linear, DNA on silica beads is used to further exclude any remaining linear DNA that escapes the exonuclease digestion (FIG. 2A). To verify the sensitivity of eccDNA detection, vertical agarose gel electrophoresis was used and as low as 5-10 picogram DNA/band could be visualized after SYBR Gold staining (FIG. 2B). Using this three-step purification procedure, purified eccDNA was then collected from 10 million HeLa cells growing at confluence, a stress condition known to increase eccDNA abundance¹⁵. The purified eccDNAs exhibited a discrete banding pattern when visualized by agarose gel electrophoresis (FIG. 1B). Furthermore, mtDNA was also efficiently removed by the treatment of PacI restriction digestion (FIG. 1B, compare lanes 2 and 3). The circularity of purified eccDNAs was confirmed using scanning atomic force microscope (SAFM)¹⁹ (FIG. 1C). To determine whether eccDNAs are present in non-cancer cells, the same procedures were applied to mouse embryonic stem cells (mESCs). The purified eccDNAs from mESCs exhibited a similar banding pattern (FIG. 1D), and their purity and circularity were also verified by SAFM (FIG. 1E).

### Example 2—EccDNAs are Mapped to Widespread Locations Across the Entire Genome

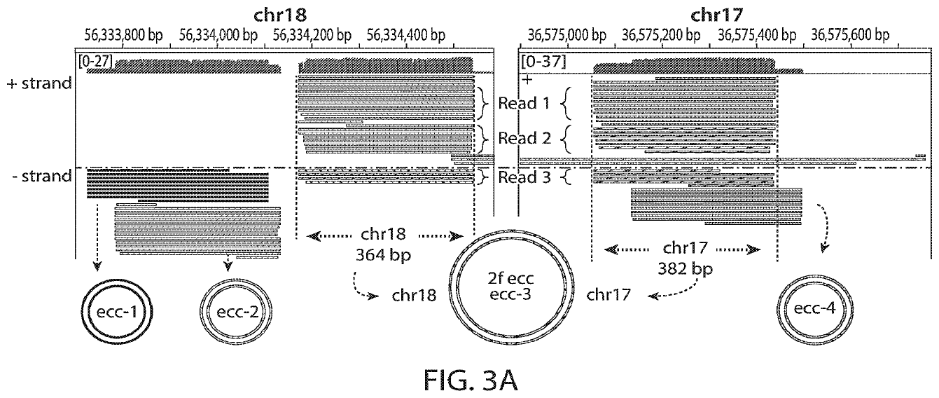

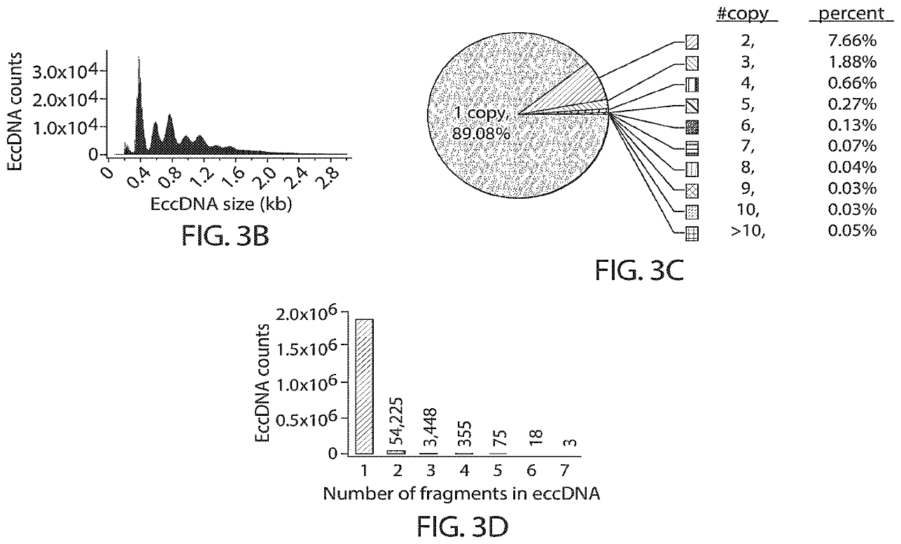

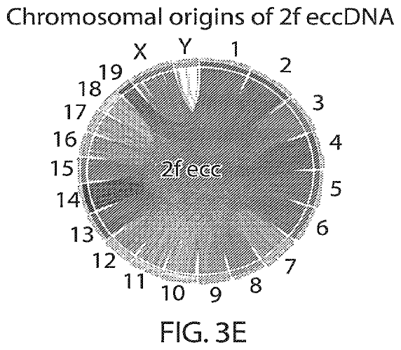

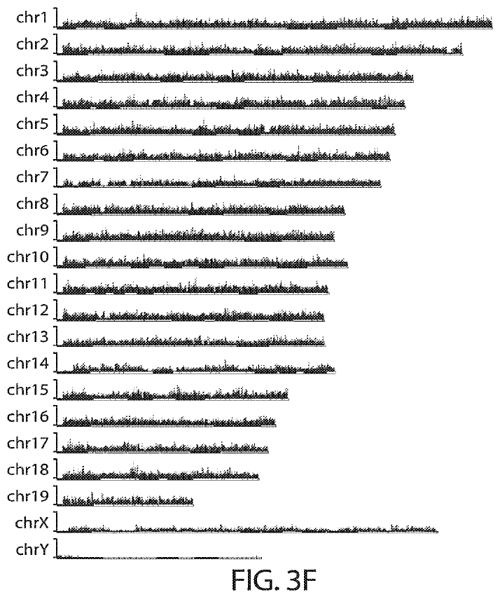

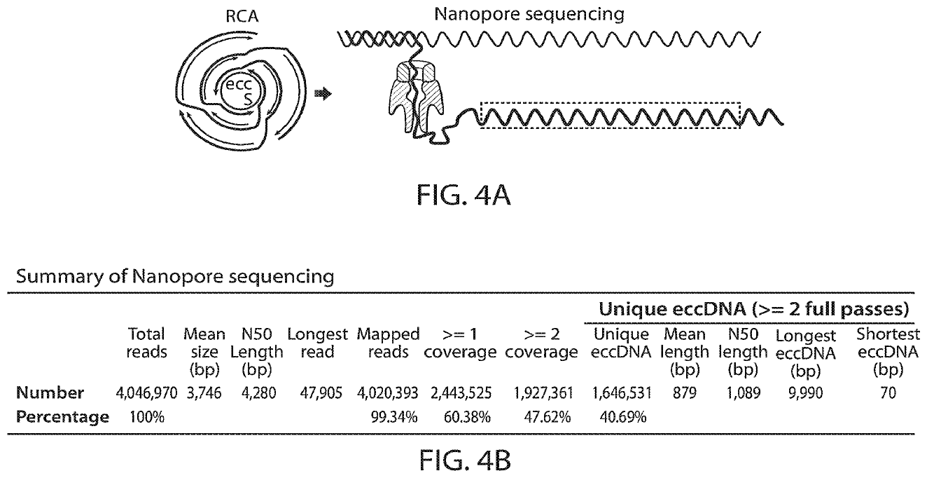

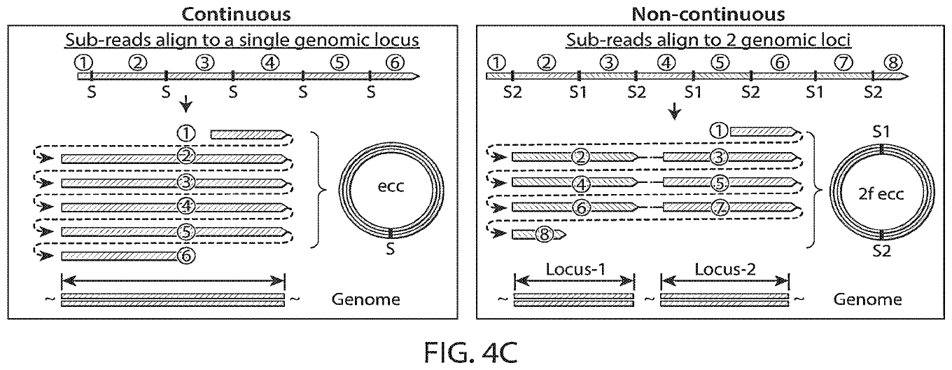

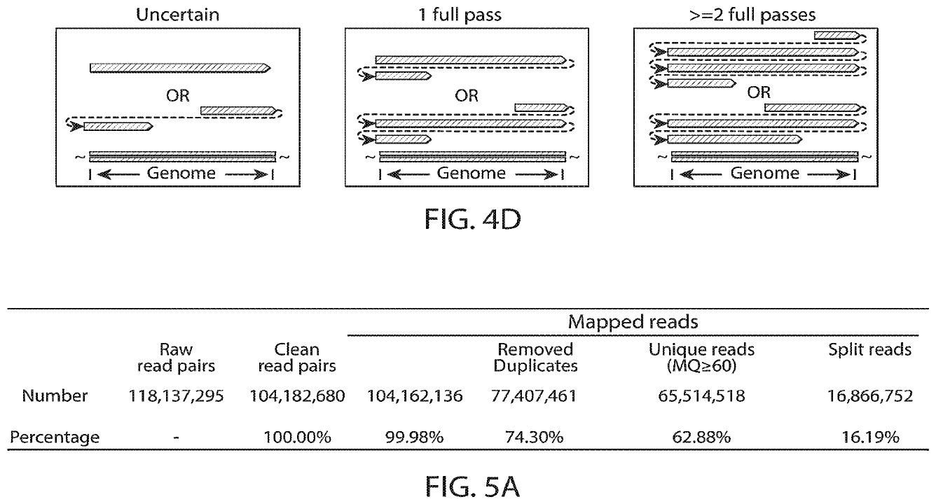

To gain insights into the potential mechanism of eccDNA biogenesis, the genomic source of eccDNAs was investigated. HeLa cells are notorious for their aberrant genome, including aneuploidy and numerous structural variations, such as deletions, duplications, inversions, translocations and rearrangements, etc.²⁰, making interpretation of sequencing data and dissection of the eccDNA biogenesis mechanism difficult. Therefore, eccDNA was sequenced and mapped using mESCs, which maintain their genetic integrity during cell culture²¹. To this end, rolling cycle amplification (RCA) was first performed, then amplified eccDNAs were sequenced using Nanopore sequencing (FIG. 1A). Nanopore sequencing has advantages over Illumina short-read sequencing for retrieving full-length sequences, as the RCA converted multi-repeat eccDNA can be sequenced as a single long molecule by Nanopore sequencing (FIG. 4A). Importantly, repeated sequencing of the same allows for the generation of a consensus sequence that matches the full-length sequence of the original eccDNA (FIG. 3A, FIG. 4C) without further in silico inference, which is often the case when utilizing short read sequencing. A total of ˜4 million long reads were obtained with a mean size of 3.7 kb (FIG. 4B). A computational threading method was employed to directly identify full-length eccDNA, based on the aligned tandem copies of eccDNA molecule in each of the Nanopore long reads (FIG. 3A). To reduce false positives due to RCA artifacts and sequencing errors due to the Nanopore technique²², the 1.9 million long-reads were only used to identify high confidence eccDNAs wherein each contained at least two full passes of the same eccDNA (FIG. 4B). Unexpectedly, as many as 1.6 million unique eccDNAs with a median size of 1 kb were detected (FIG. 4B). Interestingly, the eccDNAs exhibit a regular 188 bp average size interval (FIG. 3B), and the great majority (89%) of unique eccDNAs were sequenced from a single long read (single-event eccDNA), and less than 1.5% of unique eccDNAs were sequenced from more than three unique long molecules (FIG. 3C) and no dominant eccDNA was identified. Such large numbers of single-event eccDNAs coupled with the lack of dominant eccDNA molecules suggest that the eccDNAs are unlikely derived from specific regions of the genome.

Genome mapping revealed that eccDNAs can be aligned to the genome with a variety of patterns, including adjacent, overlapped, nested, or even across different chromosomes (FIG. 3A). The great majority of eccDNAs originated from a single continuous genomic locus (Continuous eccDNA), while only a relatively very small number of eccDNAs were formed by multiple genomic fragments (Non-continuous eccDNA) (FIG. 3D, FIG. 4C). A total of 3 eccDNAs were detected that each contain 7 genomic fragments joined together to form a circle (7f eccDNA) (FIG. 3D). To determine whether the physical distance of genomic fragments affects the frequency of eccDNA formation, the genomic originations of eccDNAs containing 2 genomic fragments (2f eccDNA) was investigated. A circle plot clearly shows that paired fragments of 2f eccDNAs are not restricted to the same chromosome (FIG. 3E), but rather randomly bridged between chromosomes, indicating that eccDNAs can be formed by joining genomic fragments from any two different chromosomes. Consistently, genome mapping of all eccDNAs revealed that eccDNAs are widespread across the entire genome (FIG. 3F).

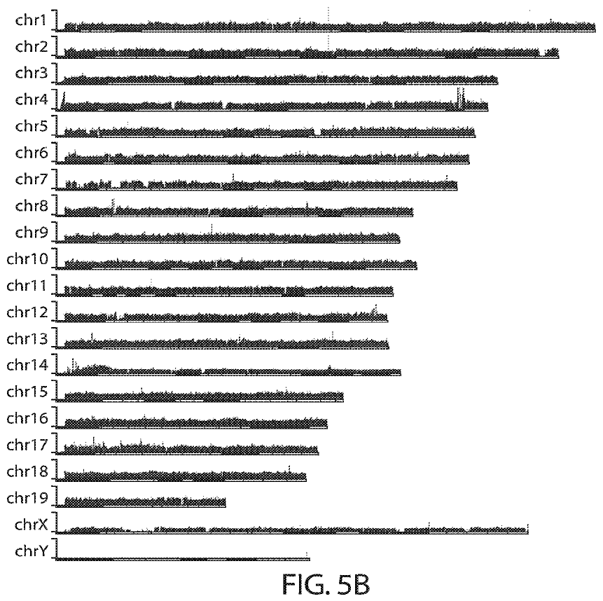

To rule out potential biases caused by uneven amplification by RCA²³, a second batch of eccDNAs was purified and directly tagged with transposon Tn5 without RCA before performing Illumina sequencing (FIG. 1A, FIG. 5A). EccDNA sequences obtained in this way should faithfully reveal their genomic location and relative abundance. Consistent with the Nanopore sequencing results, Illumina sequencing revealed widespread alignment of eccDNAs across the entire genome (FIG. 5B). The eccDNA density on X chromosome was about half of that of autosomes (FIG. 3F, FIG. 5B), which is consistent with the fact that diploid male genome of mESC/E14 cells only carry one copy of X chromosome but two copies of autosomes (FIG. 3F, FIG. 5B). The apparent lack of eccDNAs mapped to the Y chromosome is largely due to the many undetermined sequences and repeat sequences in the Y chromosome²⁴. Collectively, these data suggests that eccDNAs are widespread across the entire genome and their abundances are correlated with genomic copy numbers.

### Example 3—Apoptotic DNA Fragmentation is Required for eccDNA Generation

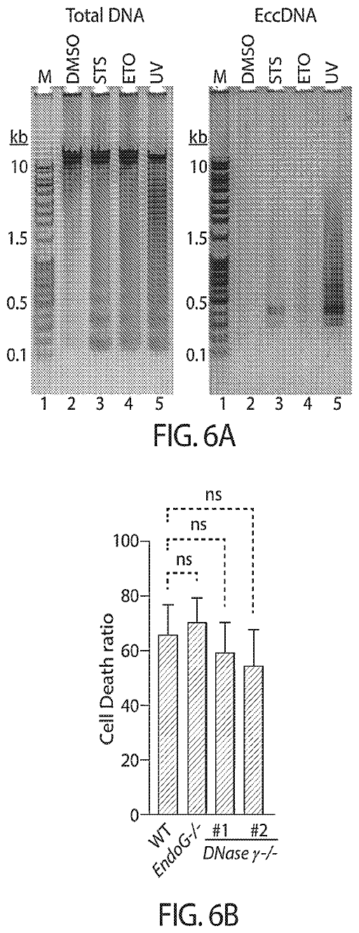

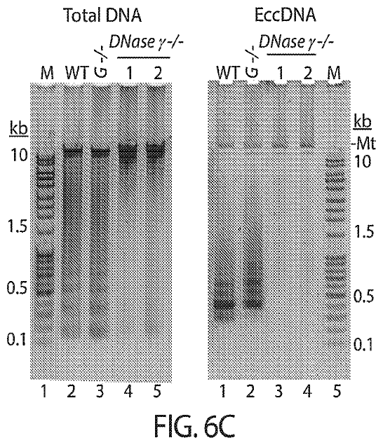

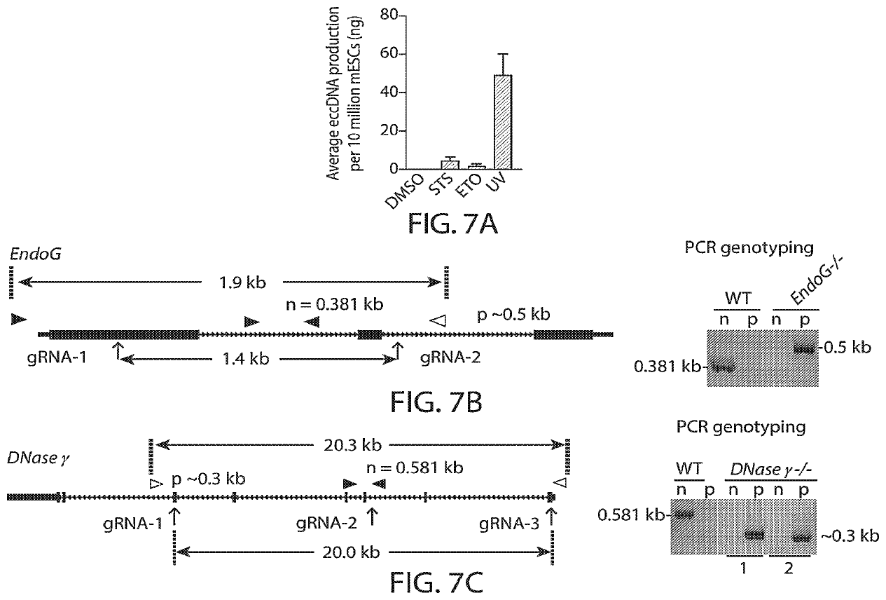

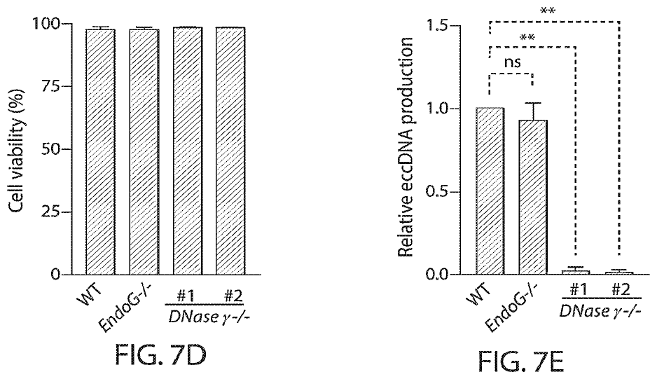

The great diversity, randomness, and size of nucleosome “ladders” (FIGS. 3A-3F) suggest that eccDNAs are generated by random ligation (including self-ligation) of nucleosome-size genomic DNA fragments. Oligonucleosomal DNA fragmentation, which can be visualized as a “ladder” of bands by agarose gel electrophoresis, is a known feature of apoptosis²⁴. To determine if apoptotic cells are the source of eccDNAs, mESCs were treated with classic apoptosis inducers, staurosporine (STS), etoposide (ETO) or UV-light to induce apoptosis. DNA agarose gel electrophoresis confirmed successful induction of apoptosis as indicated by the typical nucleosome “ladder” pattern of genomic DNA (FIG. 6A). When equal amounts of control (DMSO) and treated cells were subjected to the 3-step eccDNA purification procedure and resolved by agarose gel electrophoresis, all three treatments induced eccDNA generation compared to the control, however UV treatment displayed the strongest induction (FIG. 6A, FIG. 7A).

To determine whether DNA fragmentation during apoptosis is a prerequisite for eccDNA production, the effect of genetically blocking apoptotic DNA fragmentation (ADF) was investigated. Three nucleases, Caspase activated DNase (CAD)²⁵, Endonuclease G (EndoG)²⁶ and DNase γ²⁷ have been previously reported to degrade nuclear DNA to oligonucleosomal fragments in a cell type-specific manner. Since the nuclease responsible for mESC ADF is unknown, EndoG and DNase γ knockout mESCs were produced using a CRISPR/Cas9 targeting method (FIGS. 7B-7C). These genetic manipulations did not alter cell viability under normal culture conditions (FIG. 7D) or under UV treatment (FIG. 6B). However, in mESC cells having the DNase γ knockout, but not the EndoG knockout, ADF was largely abrogated, as indicated by the lack of a conspicuous “ladder” pattern by agarose gel electrophoresis (FIG. 6C). Purification of eccDNAs from these UV-treated, DNase γ knockout cells demonstrated that abrogation of ADF prevented eccDNA generation (FIG. 6C, FIG. 7E). For this investigation, PacI digestion was deliberately omitted to retain mitochondrial DNA (mtDNA) as an internal control for equal cell input and circular DNA recovery (FIG. 6C). These results demonstrate that ADF is a prerequisite for eccDNA generation.

### Example 4—Lig3 is the Main DNA Ligase for eccDNA Generation

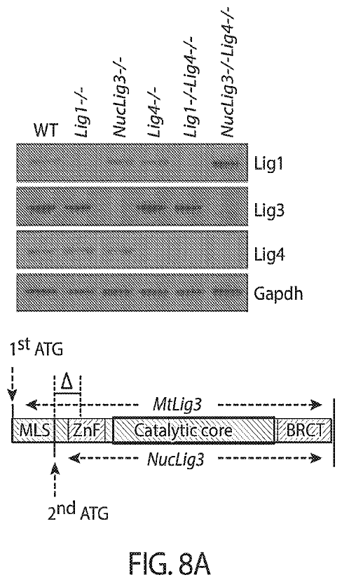

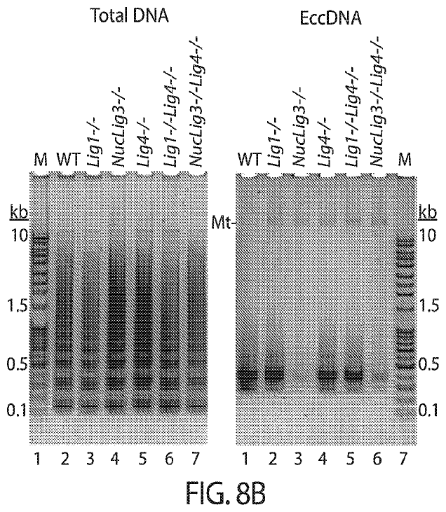

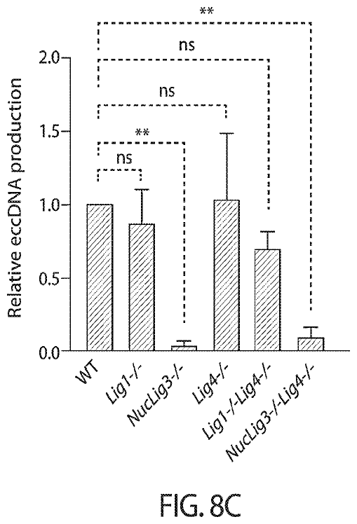

Next, the DNA ligase responsible for circularizing the fragmented DNA was identified. Mammals have three DNA ligase genes named Lig1, Lig3 and Lig4²⁸. Each ligase has specific functions and also function redundantly in DNA metabolism²⁸. The functions of these ligases have been well studied in the CH12F3 mouse B lymphocyte cell line²⁹. To determine which of these three DNA ligases is responsible for the ADF circularization, each individual DNA ligase and combinations of ligases were knocked out in CH12F3 cell by using the CRISPR/Cas9 technique and confirmed by Western blotting (FIG. 8A). Lig3 has both nuclear and mitochondrial isoforms, and the later isoform is essential for mitochondria maintenance and consequently cell viability³⁰. Thus, the Lig3 knockout cell line was generated by specifically targeting the nuclear isoform (NucLig3−/−) using CRISPR/Cas9 without interfering the mitochondrial isoform (MtLig3, FIG. 8A, lower panel). Equal numbers of WT and mutant cells were treated with staurosporine to induce apoptotic DNA fragmentation and eccDNAs were purified and visualized in an agarose gel (FIG. 8B). The results indicated that knockout of Lig1 or Lig4 alone or their combinations did not significantly affect eccDNA generation. In contrast, the Lig3 knockout greatly affected eccDNA generation (FIGS. 8B-8C). Since the double knockout of Lig1 and Lig3 is cell lethal^{29,30}, it's unknown if the Lig1 and Lig3 double knockout can completely abrogate eccDNA generation. Nevertheless, these data indicates that Lig3 is the main ligase responsible for eccDNA generation in CH12F3 cells.

### Example 5—EccDNAs are Potent Innate Immunostimulants

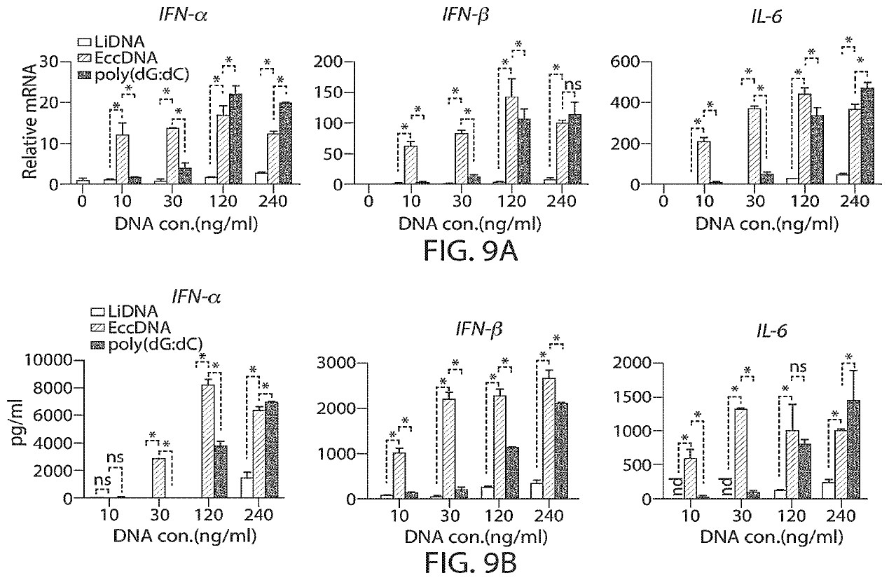

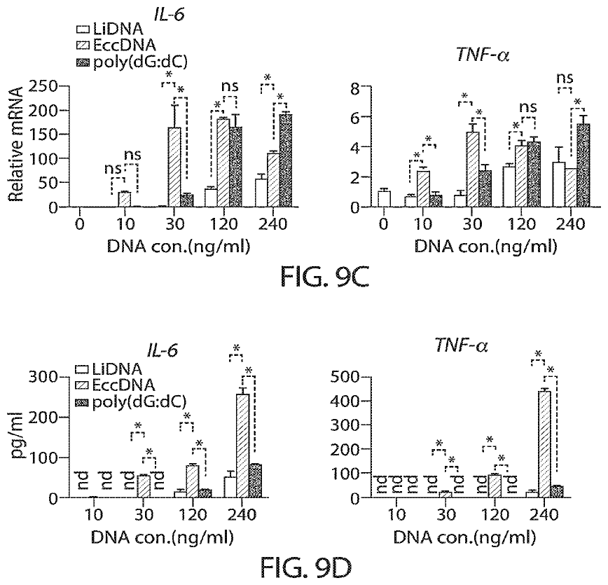

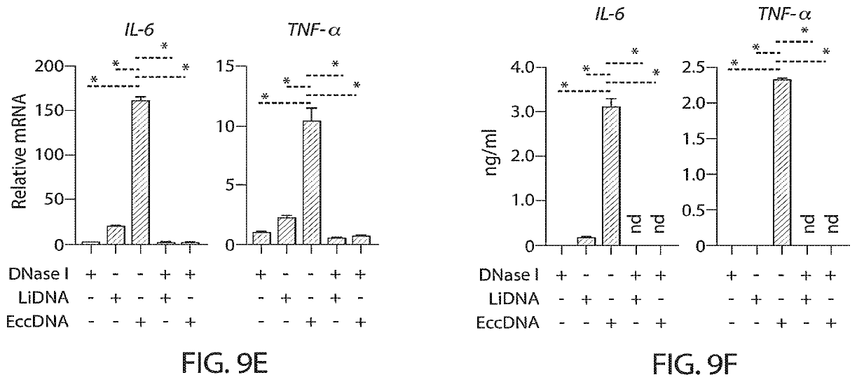

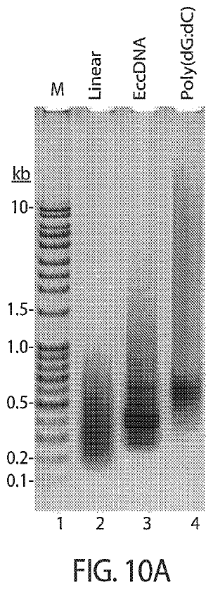

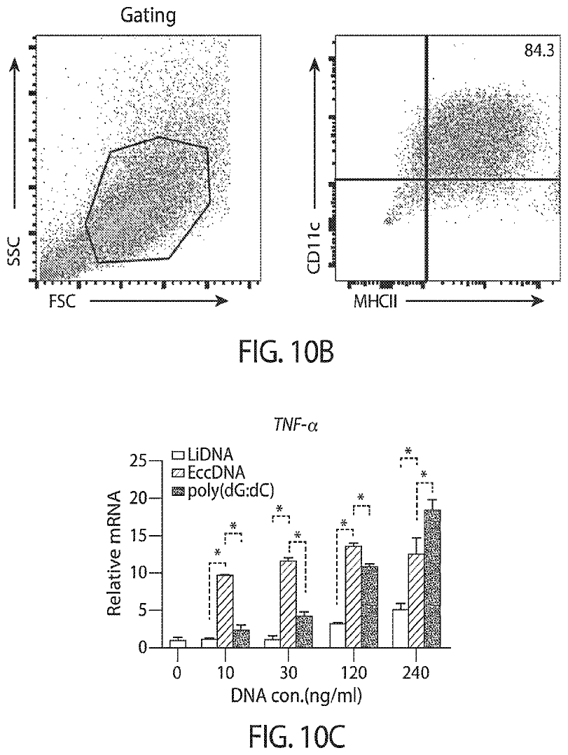

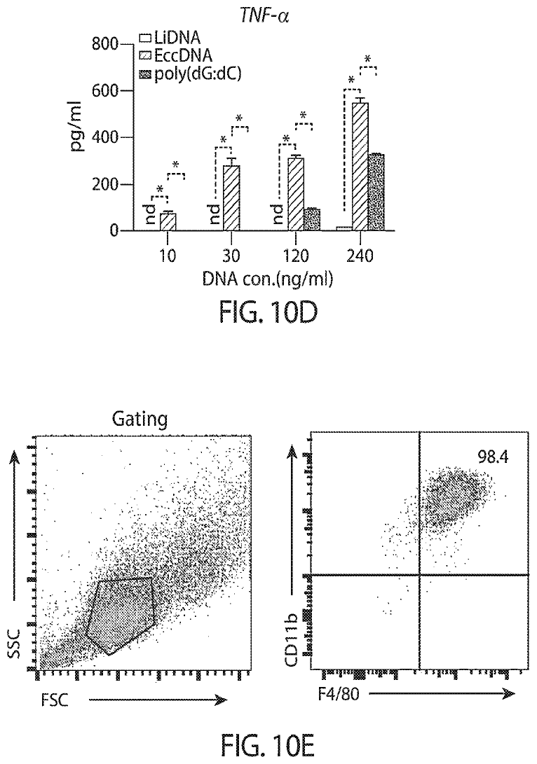

The above results demonstrate that eccDNAs are ligation products of fragmented genomic DNA of apoptotic cells. DNA released from dying cells has been previously reported to promote immune responses^{31,32}. Two important mediators of immune response, Toll-like receptor 9 (TLR9)³³ and high mobility group box 1 (HMGB1)³⁴ have been reported to preferentially bind to curved DNA structures. These observations suggested investigating the possibility that eccDNAs act as immunostimulants. To this end, bone marrow derived dendritic cells (BMDCs) were selected for investigation not only because BMDCs can be easily prepared by co-culturing bone marrow (BM) cells with Granulocyte-macrophage colony-stimulating factor (GM-CSF)³⁵, but also because dendritic cells (DCs) are professional phagocytotic cells involved in immune-responses³⁶ ENREF 32. To determine if eccDNA is specifically capable of triggering immune responses, sheared linear genomic DNA (Linear DNA) with sizes similar to that of eccDNAs, as well as the potent and widely used DNA ligand for cytosolic DNA sensors, poly(dG:dC)³⁷, were also included in parallel experiments (FIG. 10A). After confirming successful BMDC differentiation (FIG. 10B), different amounts of DNAs were transfected into BMDCs. Twelve hours after transfection, cells were harvested for mRNA extraction and quantitative reverse transcription polymerase chain reaction (RT-qPCR). The results show that, compared to the control (mock transfection), type I interferons (IFN-α, IFN-β), interleukin IL-6, and tumor necrosis factor (TNF-α) were all significantly induced by eccDNAs at a wide range of concentrations (10-240 ng/mL) (FIG. 9A, FIG. 10C). Surprisingly, the widely regarded “potent” cytokine inducer poly(dG:dC) is not as nearly potent as eccDNAs at lower concentrations, and linear DNA only triggered a mild response even at the highest concentration, indicating that dendritic cells are much more sensitive to eccDNA treatment than that of linear DNA and poly(dG:dC) (FIG. 9A, FIG. 10C). Consistent with the RT-qPCR results, enzyme-linked immunosorbent assay (ELISA) confirmed the strong potency of eccDNAs in cytokine induction (FIG. 9B, FIG. 10D).

In addition to dendritic cells, macrophages are also known to respond to immunostimulants³⁸. To determine whether eccDNA can also activate macrophages for cytokine induction, bone marrow derived macrophages (BMDMs) were also generated and assayed (FIG. 10E) by following an established method³⁹. Similar to what was observed in experiments with BMDCs, eccDNAs can also activate BMDMs to induce cytokine production although the fold change of activation is generally lower than that of BMDCs (FIGS. 9C-9D). Nevertheless, eccDNAs are still more potent than poly(dG:dC), particularly at lower concentrations (10, 30 ng/mL). These data indicates that eccDNAs are very potent immunostimulants in activating both BMDCs and BMDMs. To demonstrate that eccDNAs, but not another material that co-purifies with them, are indeed responsible for BMDC activation, purified eccDNAs were pretreated with DNase I before transfection. Pretreatment completely abrogated the ability of eccDNA to activate BMDC for cytokine induction (FIGS. 9E-9F), indicating that eccDNA is responsible for BMDC activation.

### Example 6—Circularization, but not Sequence, Confers eccDNA Immunostimulatory Activity

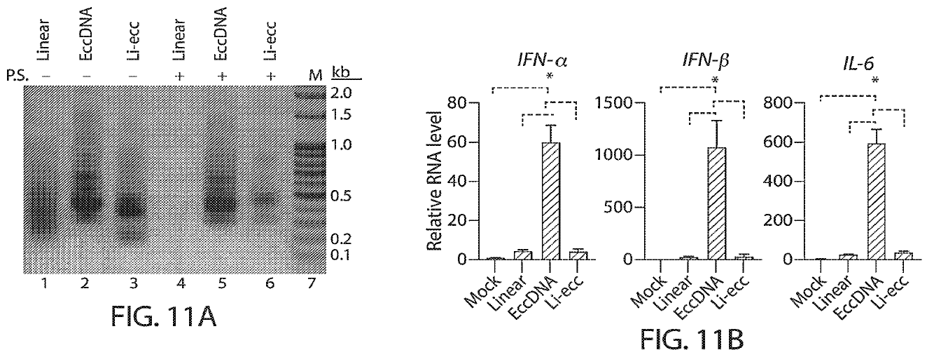

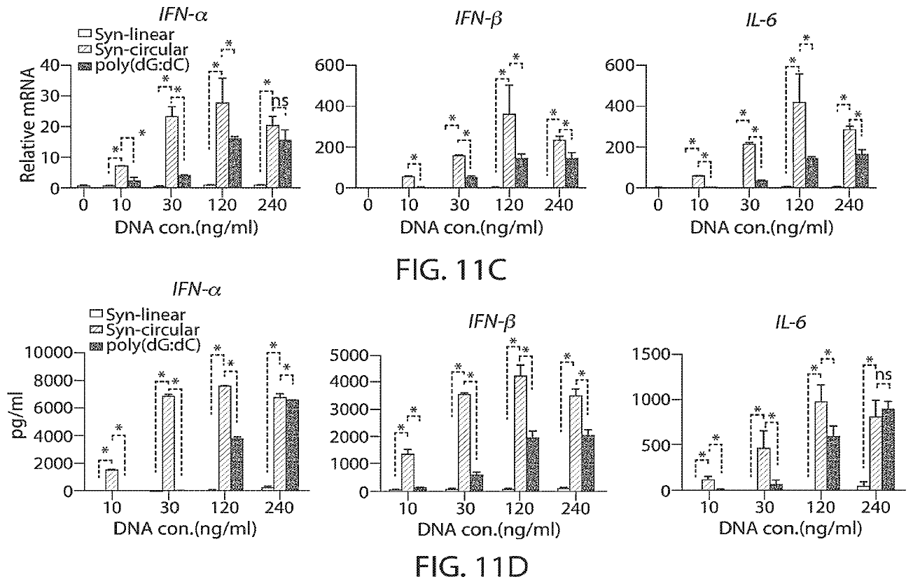

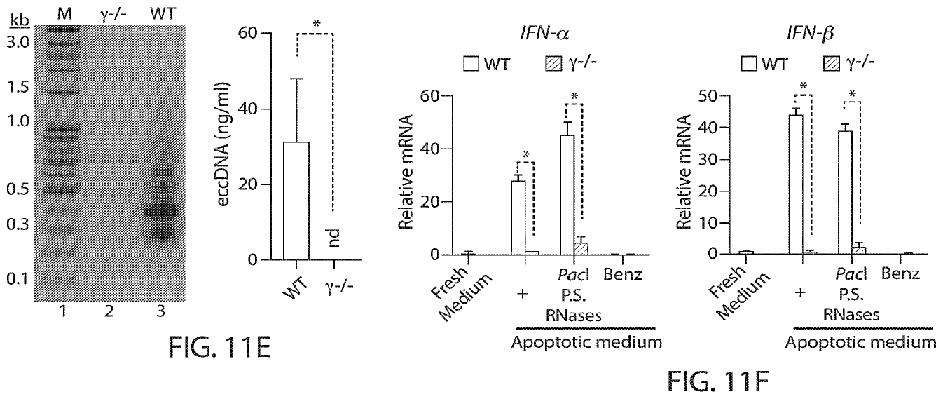

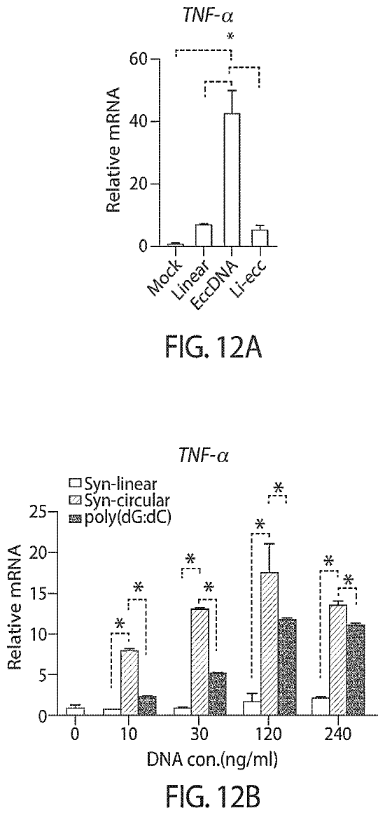

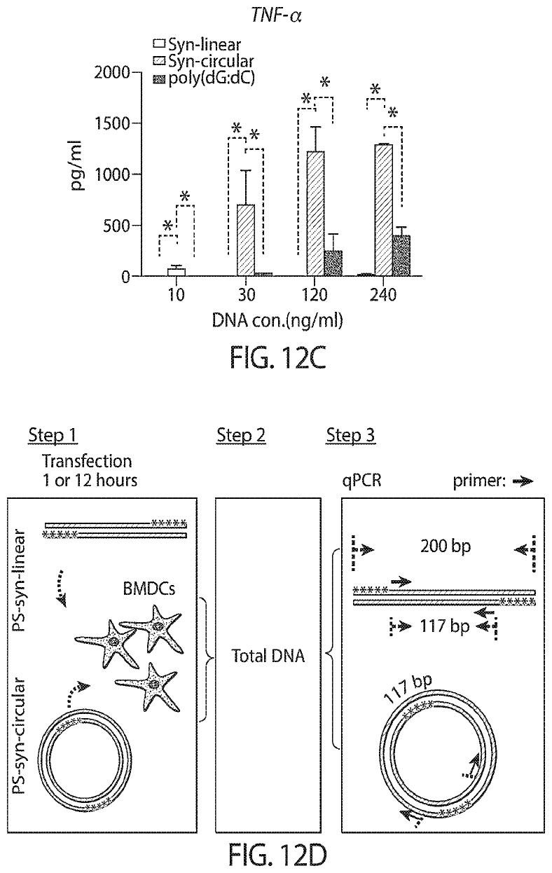

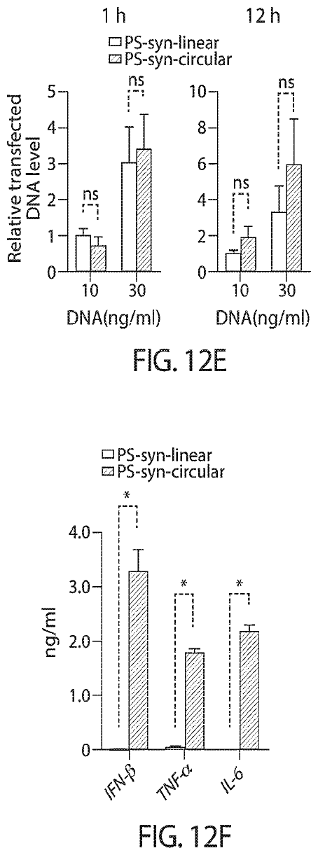

To determine whether the circular nature of eccDNAs is critical for their strong immunostimulatory activity, it was investigated whether linearization of eccDNAs would abolish their immunostimulatory activity. To this end, purified eccDNAs were first treated with FnoCas12a (Cpf1), which is capable of introducing one nick per circular DNA in the presence of Mn²⁺, but in the absence of guide RNA⁴⁰. The nicked eccDNAs were then treated with single strand specific endonuclease Nuclease S1, which cleaves the intact circular strand at the site opposite to the nick to generate linearized eccDNA (Li-eccDNA). The linearization of eccDNAs was confirmed by their sensitivity to exonuclease digestion, while intact eccDNAs are resistant (FIG. 11A, compare lanes 5 and 6). When equal amounts of linear DNAs, eccDNAs, and Li-eccDNAs were transfected to BMDCs, Li-eccDNAs behaved like linear DNAs and failed to activate IFN-α, IFN-β, IL-6, and TNF-α (FIG. 11B, FIG. 12A). This result demonstrated that the circular nature of eccDNAs is critical for their strong immunostimulant activity. Given that eccDNAs are derived from randomly ligated genomic fragments, their sequences are unlikely to significantly contribute to their potency. To test this notion, a synthetic circular DNA and its linear counterpart were generated with 200 bp of random DNA sequence and transfected into BMDCs to compare their ability to induce cytokine gene activation. Circular DNA, but not linear DNA of the same sequences, greatly induced cytokine gene transcription (FIG. 11C, FIG. 12B). Similar to native eccDNAs, synthetic circular DNA also showed higher potency in cytokine gene activation when compared to that of poly(dG:dC). Consistently, ELISA confirmed the strong cytokine induction capacity of synthetic circular DNA (FIG. 11D, FIG. 12C).

It is possible that the increased immunostimulatory potency of circular DNA could be simply due to its increased stability and transfection efficiency. To examine this possibility, and to minimize the effects of exonuclease activity on the comparison of transfection efficiency between linear DNA and circular DNA, end-protected DNA was generated by adding phosphorothioate⁴¹ on both ends of the 200 bp linear DNA (PS-Syn-linear). To exclude the potential effect of phosphorothioate bonds on transfection and immune stimulation, equal number of phosphorothioate bonds were also put in the circular counterpart (PS-Syn-circular). Both linear and circular DNAs were separately transfected to BMDCs, cell lysates and culture media were collected for qPCR and ELISA assay to compare their transfection efficiency (1 hour after transfection), stability and cytokine induction (12 hours after transfection) (FIG. 12D). There was no significant difference in the transfection efficiency or stability of 200 bp linear and circular DNAs under these experimental conditions (FIG. 12E). Yet, circular DNA induced high level of cytokine production while its linear counterpart did not (FIG. 12F). Collectively, these data support the notion that the circularity rather than the sequence of eccDNAs confers the high potency of their immunostimulant activity.

### Example 7—EccDNAs Present in Apoptotic Medium can be Sensed by BMDCs

Since eccDNAs are generated in apoptotic cells, eccDNAs are potentially released into the culture medium. Indeed, after UV treatment to induce apoptosis of mESCs, a significant amount of eccDNAs are present in the cell free supernatant of apoptotic medium (FIG. 11E). To determine if eccDNAs from the supernatant of apoptotic medium can be actively sensed by BMDCs without transfection, BMDCs were co-incubated with the cell-free apoptotic supernatant of WT or DNase γ−/− mESCs cells (DNase γ−/− cells are deficient in eccDNA generation, see FIG. 6C, FIG. 11E). RT-qPCR analysis indicates that the supernatant of apoptotic medium of WT cells, but not DNase γ−/− cells, stimulates IFN-α, and IFN-3 expression (FIG. 11F). Importantly, this stimulation is not sensitive to pre-treatment of the supernatant with plasmid safe exonuclease (linear DNA-specific), PacI restriction enzyme, and RNases that digest linear DNA, mitochondrial DNA, and RNA, respectively (FIG. 11F), but is sensitive to pre-treatment of Benzonase, a nuclease that destroy all forms of DNA and RNA without proteolytic activity (FIG. 11F). These data indicates that eccDNAs, rather than linear DNAs, mitochondrial DNAs, or RNAs in the supernatant of apoptotic medium are responsible for the induced immune response. Furthermore, this result also indicates that eccDNAs can be actively sensed by BMDCs without transfection. These results indicate that eccDNAs are potent damage-associated molecular patterns (DAMPs) of the innate immune system⁴².

### Example 8—STING is Required for eccDNA-Triggered Immune Response

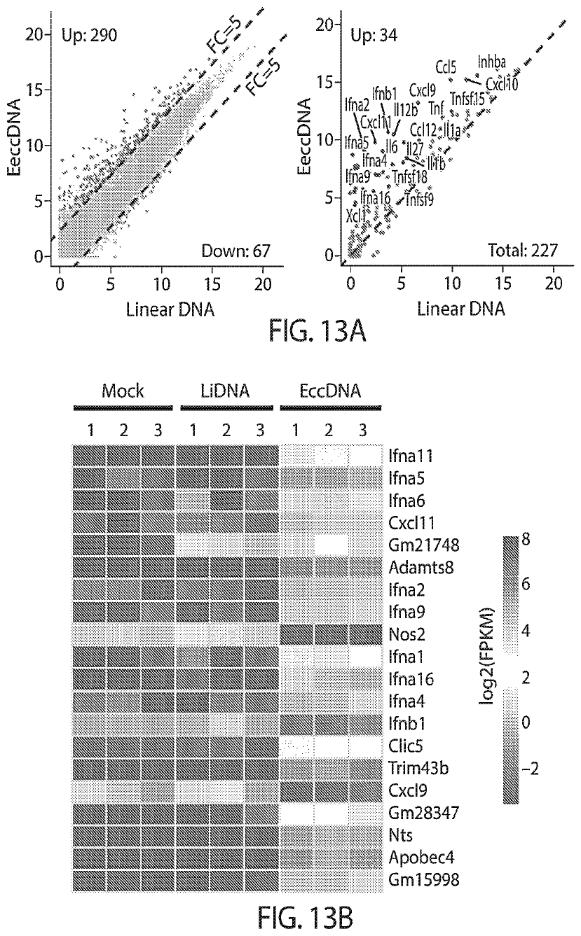

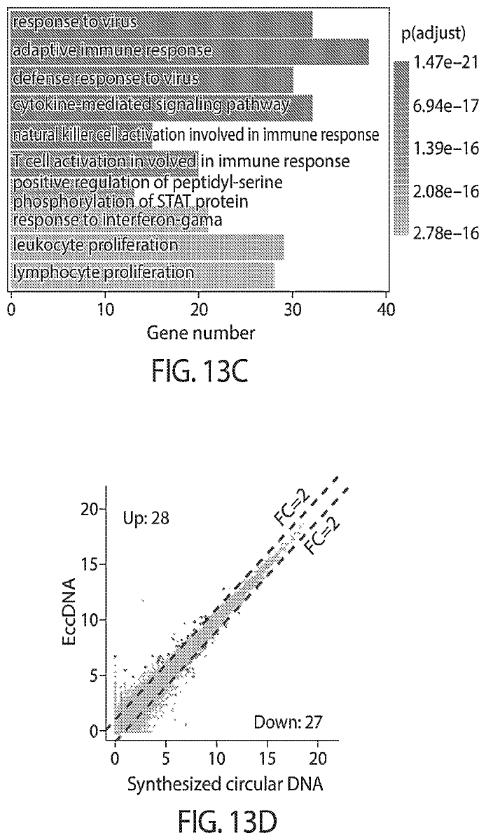

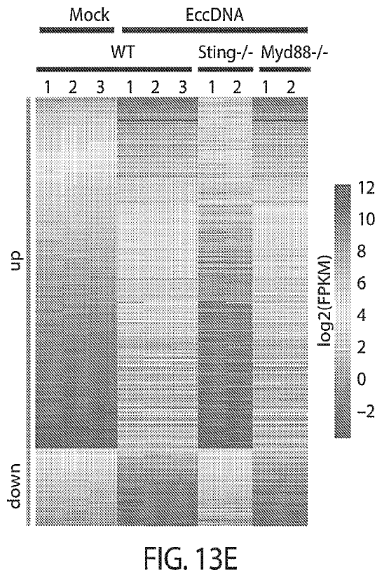

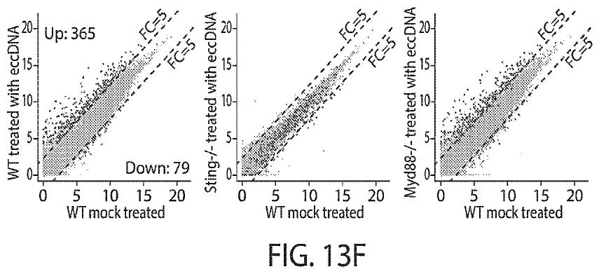

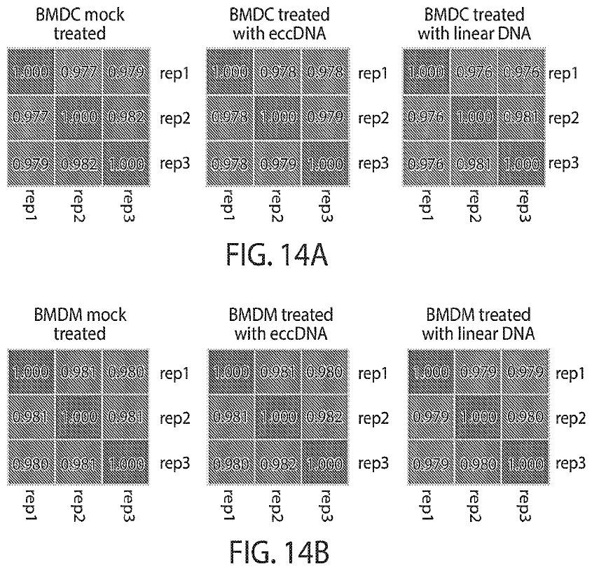

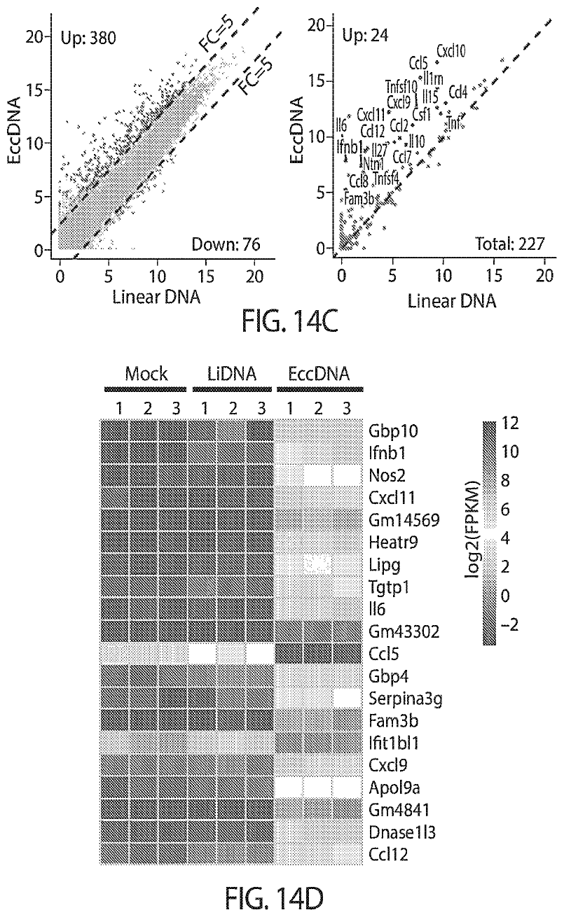

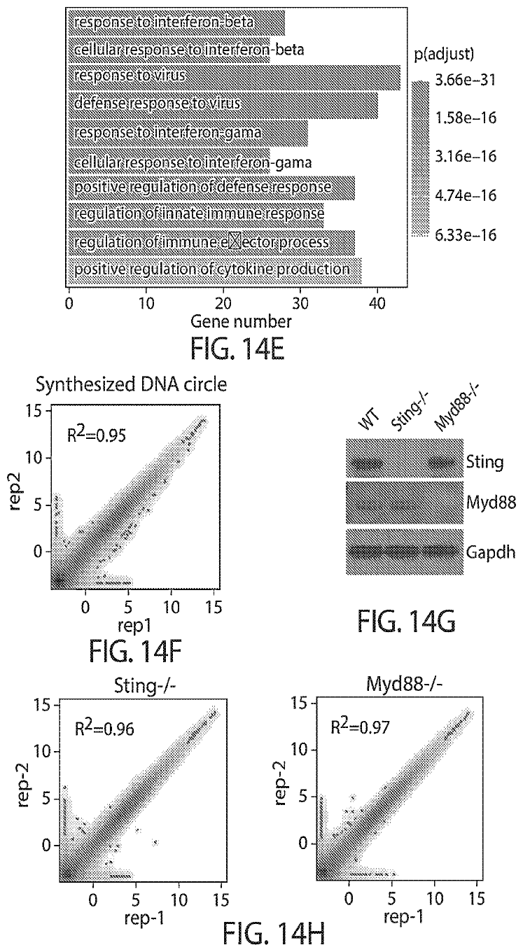

To assess the global transcriptional effect of eccDNA, RNA-seq analysis was performed of BMDC transfected with purified eccDNAs or sonicated genomic DNA of similar size (FIG. 14A). Comparative analysis indicated that eccDNAs, but not linear DNA controls, significantly increased the expression of ˜290 genes (p-value <0.001), including 34 cytokines and chemokines (Table 1, FIG. 13A), under these experimental conditions (30 ng/mL DNA transfected). Importantly, 9 of the top 20 up-regulated genes belong to type I interferons (FIG. 13B). Gene ontology (GO) enrichment analysis revealed that the up-regulated genes are enriched for terms relevant to immune response and related signaling pathways (FIG. 13C), supporting the conclusion that eccDNA is a potent innate immunostimulant that can generally increase innate immune response. Parallel experiments further demonstrated a similar effect of eccDNAs in BMDMs (Table 2, FIGS. 14B-14E). Collectively, these data support the capacity and potency of eccDNA in triggering a general immune response, as control linear genomic DNA fragments failed to trigger such responses under the same conditions (FIG. 13B). Importantly, this property of eccDNA depends on its circularization, but not its sequence, as transfection of a synthetic circular DNA with random sequences into BMDC triggered a similar transcriptional response to that of purified eccDNAs (FIG. 13D, FIG. 14F).

The sensing pathways by which eccDNAs trigger the immune response were further investigated. There are two known major DNA sensing pathways: STING (stimulator of interferon genes) and Toll-like receptors (TLR)⁴³. To determine whether either of these two DNA sensing pathways are responsible for eccDNA sensing, mouse BMDCs were generated that are deficient for STING⁴⁴ or Myd88⁴⁵ (FIG. 14G), thereby impairing either STING or the TLRs, respectively, and then subjected to transfection with eccDNA. Comparative RNA-seq analysis demonstrated that while loss of function of Myd8 does not affect the capacity of BMDC to respond to eccDNAs, loss of STING function completely abrogates the capacity of BMDC to respond to eccDNAs, as almost all the genes normally induced by eccDNA fail to be induced in the absence of STING (Table 3, FIG. 13E-13F, FIG. 14H). These data strongly suggests that the STING pathway is responsible for sensing eccDNA to mediate its immune response.

TABLE 1
Differentially expressed genes in BMDCs treated with eccDNA

| Gene: | Gene type: | Adjusted p-value: | Log₂ fold change: |
|---|---|---|---|
| Acsl1 | protein coding | 0 | 3.721330625 |
| Nos2 | protein coding | 1.17E−237 | 7.351059885 |
| Clec4e | protein coding | 3.80E−227 | 5.033091852 |
| Rgs1 | protein coding | 1.43E−224 | 2.996548671 |
| Htra4 | protein coding | 2.55E−194 | 4.239308645 |
| Serpinb9 | protein coding | 2.74E−174 | 3.722428949 |
| Tnf | protein coding | 5.06E−159 | 3.259745397 |
| Tgm2 | protein coding | 6.67E−153 | 3.172805781 |
| Dcbld2 | protein coding | 4.00E−147 | 2.534591961 |
| Tnfaip3 | protein coding | 1.79E−145 | 2.668503705 |
| Plaat3 | protein coding | 7.41E−142 | 3.117726819 |
| Cst7 | protein coding | 1.09E−140 | 4.332392814 |
| Ptgs2 | protein coding | 2.25E−136 | 5.80173601 |
| Nfkbie | protein coding | 3.37E−130 | 2.514508779 |
| Inhba | protein coding | 4.25E−120 | 3.573550314 |
| Rapgef2 | protein coding | 7.57E−120 | 3.069498864 |
| Slc7a2 | protein coding | 1.05E−119 | 2.776400222 |
| Sdc4 | protein coding | 1.49E−119 | 2.847498793 |
| Ifnb1 | protein coding | 7.32E−118 | 7.256845046 |
| Faah | protein coding | 5.83E−115 | 4.934722249 |
| Tpx2 | protein coding | 1.46E−107 | 2.796690211 |
| Tma16 | protein coding | 4.92E−106 | 2.590591479 |
| Csrnp1 | protein coding | 2.24E−105 | 2.955611455 |
| Flnb | protein coding | 3.92E−103 | 2.826845896 |
| Xkr8 | protein coding | 2.50E−102 | 3.196942892 |
| Acod1 | protein coding | 2.39E−99 | 3.851355215 |
| Tnfsf15 | protein coding | 4.50E−93 | 2.895848377 |
| Ptx3 | protein coding | 9.59E−93 | 2.539611783 |
| AA467197 | protein coding | 1.67E−91 | 2.748727273 |
| Hmgn3 | protein coding | 3.77E−83 | 3.175468951 |
| Hcar2 | protein coding | 6.81E−83 | 2.582867159 |
| Slco3a1 | protein coding | 1.48E−81 | 3.236668512 |
| Lrrc8c | protein coding | 1.60E−79 | 2.776819365 |
| Gca | protein coding | 1.57E−75 | 3.050205925 |
| Fabp3 | protein coding | 1.57E−75 | 3.041371555 |
| Il1a | protein coding | 9.97E−75 | 2.45707133 |
| Tmem171 | protein coding | 1.37E−74 | 3.380717159 |
| Morrbid | lncRNA | 6.67E−73 | 2.500107689 |
| Serpinb2 | protein coding | 2.19E−70 | 3.442918238 |
| Rnf19b | protein coding | 1.19E−69 | 2.618015063 |
| Ppp1r15a | protein coding | 2.67E−69 | 2.382877286 |
| Abcb1a | protein coding | 5.26E−68 | 3.938005355 |
| Cxcl11 | polymorphic pseudogene | 4.85E−67 | 7.969314313 |
| Gypc | protein coding | 8.64E−67 | 2.912826607 |
| Slamf8 | protein coding | 1.08E−66 | 2.516770936 |
| Adora2a | protein coding | 1.67E−65 | 2.556197164 |
| BC023105 | processed pseudogene | 9.75E−65 | 5.651262388 |
| Upp1 | protein coding | 1.73E−62 | 3.07466451 |
| Gm34643 | lncRNA | 1.31E−57 | 2.987846616 |
| Cflar | protein coding | 2.79E−57 | 2.477552693 |
| Marcksl1 | protein coding | 3.14E−57 | 3.481697909 |
| Stx11 | protein coding | 4.86E−54 | 3.385810915 |
| Gem | protein coding | 1.54E−53 | 4.072244725 |
| Plekha4 | protein coding | 5.15E−53 | 4.071138711 |
| Ccrl2 | protein coding | 1.69E−52 | 3.698004046 |
| Ccl5 | protein coding | 1.98E−52 | 5.682085629 |
| Atp13a4 | protein coding | 9.79E−52 | 3.46373729 |
| Hbegf | protein coding | 6.62E−51 | 2.590948593 |
| Flt4 | protein coding | 1.98E−49 | 3.850433203 |
| Gm13571 | lncRNA | 2.57E−49 | 3.230562992 |
| Akap12 | protein coding | 6.70E−49 | 3.010003898 |
| H2-Q6 | protein coding | 6.86E−48 | 3.57159294 |
| Il6 | protein coding | 1.77E−47 | 6.152232135 |
| A530040E14Rik | lncRNA | 2.34E−47 | 3.21481822 |
| Pla1a | protein coding | 3.91E−46 | 4.730128577 |
| Il12rb1 | protein coding | 1.69E−45 | 4.294282396 |
| Il1b | protein coding | 2.72E−45 | 3.173651013 |
| Bambi-ps1 | processed pseudogene | 5.98E−45 | 3.731584675 |
| H2-Q4 | protein coding | 1.38E−44 | 2.362676823 |
| Sco1 | protein coding | 3.89E−44 | 2.519292574 |
| Scimp | protein coding | 1.02E−43 | 2.649300611 |
| Gbp11 | polymorphic pseudogene | 3.15E−43 | 3.138191968 |
| 4933412E12Rik | lncRNA | 2.45E−42 | 2.63584167 |
| Tnfsf18 | protein coding | 1.09E−41 | 3.061359567 |
| Ifi214 | protein coding | 1.26E−41 | 2.721930756 |
| Tnn | protein coding | 1.30E−41 | 5.455562539 |
| C130026I21Rik | protein coding | 7.92E−41 | 2.625122775 |
| Vcan | protein coding | 5.36E−40 | 3.296502545 |
| H2-Q7 | protein coding | 1.81E−39 | 3.191943288 |
| Kalrn | protein coding | 1.91E−38 | 3.452049611 |
| Armcx6 | protein coding | 3.55E−38 | 3.943882491 |
| Fgfbp3 | protein coding | 4.85E−38 | 2.605897995 |
| Cd38 | protein coding | 4.71E−37 | 2.661484488 |
| Dmtn | protein coding | 7.80E−37 | 3.613626087 |
| Col27a1 | protein coding | 2.37E−36 | 3.496499052 |
| Plagl1 | protein coding | 3.11E−36 | 4.675020917 |
| Mir155hg | lncRNA | 9.46E−36 | 3.623543573 |
| Hdc | protein coding | 1.35E−35 | 3.510331352 |
| Nlgn2 | protein coding | 4.77E−35 | 2.57065325 |
| Wnk2 | protein coding | 6.18E−35 | 2.647175751 |
| Angpt1 | protein coding | 1.04E−34 | 4.890919539 |
| Apol9b | protein coding | 1.05E−34 | 3.487565987 |
| Ch25h | protein coding | 4.42E−33 | 5.19824984 |
| Gbp2 | protein coding | 5.37E−33 | 2.552675972 |
| Il23r | protein coding | 1.58E−32 | 3.624603383 |
| Traf1 | protein coding | 1.79E−32 | 2.34311983 |
| AC147806.2 | protein coding | 4.58E−32 | 2.761528958 |
| Clic5 | protein coding | 5.59E−32 | 7.113595975 |
| Zc3h6 | protein coding | 6.43E−32 | 2.456589817 |
| Carmil1 | protein coding | 1.24E−31 | 2.510250485 |
| Isg15 | protein coding | 1.30E−31 | 2.599930775 |
| Gm47662 | lncRNA | 1.36E−31 | 5.202979875 |
| Ifna2 | protein coding | 1.52E−31 | 8.80823046 |
| Lta | protein coding | 1.24E−30 | 4.301966286 |
| Gm4841 | protein coding | 1.39E−30 | 5.987795332 |
| Il18bp | protein coding | 3.39E−30 | 2.626237812 |
| Abtb2 | protein coding | 5.41E−30 | 2.694789077 |
| Gnb4 | protein coding | 1.21E−29 | 2.32877643 |
| Gm21860 | lncRNA | 1.49E−29 | 2.610555506 |
| Slc6a19 | protein coding | 1.76E−29 | 4.034527897 |
| Irf7 | protein coding | 3.10E−29 | 2.681358583 |
| Ctla2b | protein coding | 4.56E−29 | 3.149633058 |
| Enpp4 | protein coding | 6.48E−29 | 2.785494496 |
| Gpr55 | protein coding | 1.05E−28 | 2.694982199 |
| Pakap | protein coding | 1.12E−28 | 2.458771779 |
| Mtus1 | protein coding | 1.48E−28 | 2.584745232 |
| Cd86 | protein coding | 9.66E−28 | 2.654508432 |
| Slc17a8 | protein coding | 1.24E−27 | 2.492104948 |
| Cxcl9 | protein coding | 1.68E−27 | 6.742235394 |
| Il12b | protein coding | 5.49E−27 | 6.418842131 |
| Asap3 | protein coding | 5.77E−27 | 4.479328787 |
| Rnf225 | protein coding | 8.46E−27 | 7.379903955 |
| A630012P03Rik | lncRNA | 1.39E−26 | 3.785038363 |
| Uaca | protein coding | 2.18E−26 | 2.353404912 |
| Gdf11 | protein coding | 2.41E−26 | 4.712358337 |
| Sdcbp2 | protein coding | 3.76E−26 | 2.79926251 |
| Gm16685 | lncRNA | 4.56E−26 | 3.586502158 |
| Socs1 | protein coding | 2.38E−25 | 2.649581388 |
| Ubd | protein coding | 3.50E−25 | 4.91040206 |
| Hspa1a | protein coding | 5.97E−25 | 2.85912107 |
| Batf2 | protein coding | 6.16E−25 | 2.505852678 |
| Glrp1 | protein coding | 1.11E−24 | 2.374354824 |
| Sell | protein coding | 2.88E−24 | 2.643786547 |
| Gm7609 | protein coding | 4.07E−24 | 3.176110724 |
| Lipg | protein coding | 5.54E−24 | 2.8187411 |
| Oprd1 | protein coding | 7.25E−24 | 3.746318034 |
| Pou3f1 | protein coding | 7.69E−24 | 3.260374314 |
| Pde11a | protein coding | 1.44E−23 | 2.686362063 |
| Usp18 | protein coding | 1.88E−23 | 2.386946605 |
| Lhx2 | protein coding | 8.06E−23 | 3.857127146 |
| Gpr33 | protein coding | 9.77E−23 | 4.320597277 |
| Ifna6 | protein coding | 1.33E−22 | 6.108359651 |
| Hspa1b | protein coding | 1.49E−22 | 2.732669952 |
| Ccl8 | protein coding | 3.55E−22 | 4.6627705 |
| Jam2 | protein coding | 3.93E−22 | 3.987105316 |
| Gm47242 | lncRNA | 5.62E−22 | 3.21310001 |
| Adhfe1 | protein coding | 9.05E−22 | 2.333173769 |
| Gm10553 | lncRNA | 1.33E−21 | 3.416918454 |
| Ccdc173 | protein coding | 1.71E−21 | 2.602410333 |
| Serpina3g | protein coding | 2.29E−21 | 4.938923964 |
| Serpinb6b | protein coding | 3.47E−21 | 2.359312448 |
| Slc28a2 | protein coding | 7.48E−21 | 2.608381632 |
| Arid5a | protein coding | 1.18E−20 | 2.375971608 |
| Rilpl1 | protein coding | 1.57E−20 | 2.329590948 |
| Clcn1 | protein coding | 2.38E−20 | 2.540453737 |
| Phf11b | protein coding | 3.51E−20 | 2.346131987 |
| 9130015A21Rik | lncRNA | 6.97E−20 | 3.102738223 |
| Trp53i11 | protein coding | 9.13E−20 | 2.917455499 |
| A530032D15Rik | protein coding | 1.50E−19 | 2.677823868 |
| Isg20 | protein coding | 3.20E−19 | 3.691862685 |
| B230303A05Rik | transcribed processed | 6.54E−19 | 3.960542571 |
| | pseudogene | | |
| Gm38025 | lncRNA | 1.59E−18 | 3.586832571 |
| Olfr56 | protein coding | 1.69E−18 | 2.394231164 |
| Heatr9 | protein coding | 1.87E−18 | 4.820333517 |
| Cldn23 | protein coding | 4.16E−18 | 4.469757053 |
| Ifna5 | protein coding | 4.99E−18 | 10.23849776 |
| Spta1 | protein coding | 7.68E−18 | 5.809418157 |
| Serpina3f | protein coding | 3.89E−17 | 4.886925015 |
| Phf11 | unprocessed pseudogene | 6.38E−17 | 2.707267289 |
| 1110002J07Rik | lncRNA | 6.46E−17 | 3.500343943 |
| Neb | protein coding | 1.45E−16 | 3.907012447 |
| Dnase1l3 | protein coding | 2.93E−16 | 5.330541551 |
| Timd4 | protein coding | 3.11E−16 | 4.476047437 |
| Tspan33 | protein coding | 4.21E−16 | 2.534724896 |
| Gm43814 | TEC | 1.15E−15 | 3.448040896 |
| Il27 | protein coding | 1.29E−15 | 4.639830407 |
| A330040F15Rik | lncRNA | 1.67E−15 | 3.102394745 |
| Oasl1 | protein coding | 1.78E−15 | 3.316792417 |
| Cd40 | protein coding | 1.84E−15 | 4.503162187 |
| H2-Q5 | polymorphic pseudogene | 1.91E−15 | 2.376332465 |
| Plat | protein coding | 2.39E−15 | 4.505536762 |
| Ddx4 | protein coding | 4.17E−15 | 2.986052767 |
| Gm11992 | protein coding | 4.57E−15 | 3.841326622 |
| Ifna4 | protein coding | 8.37E−15 | 8.257907246 |
| Slfn4 | protein coding | 8.85E−15 | 3.622137959 |
| Spata31d1b | protein coding | 1.68E−14 | 4.134188318 |
| Il15ra | protein coding | 2.47E−14 | 3.199866966 |
| Gm15726 | lncRNA | 3.82E−14 | 2.417780164 |
| Ifna9 | protein coding | 3.94E−14 | 8.976168562 |
| Adgb | protein coding | 6.81E−14 | 2.66674754 |
| AC167036.1 | protein coding | 1.05E−13 | 2.520938858 |
| Cfb | protein coding | 1.28E−13 | 2.756718952 |
| Gm16181 | protein coding | 1.82E−13 | 3.029558036 |
| Gm26902 | lncRNA | 4.23E−13 | 4.584085808 |
| Apol9a | protein coding | 5.61E−13 | 4.308985528 |
| Gm6545 | processed pseudogene | 1.12E−12 | 2.892519171 |
| Gm16094 | lncRNA | 1.55E−12 | 2.571830991 |
| Gm50083 | processed pseudogene | 1.56E−12 | 2.383880443 |
| Kdr | protein coding | 2.42E−12 | 2.951427584 |
| Ms4a4c | protein coding | 2.46E−12 | 3.516810166 |
| Efna2 | protein coding | 2.58E−12 | 3.711360856 |
| Ifna1 | protein coding | 4.45E−12 | 6.391300614 |
| Ifna11 | protein coding | 1.45E−11 | 9.75208059 |
| Gbp5 | protein coding | 1.46E−11 | 3.048846082 |
| Ccl12 | protein coding | 2.52E−11 | 3.354024441 |
| Misp | protein coding | 2.56E−11 | 2.423581647 |
| Ifi205 | protein coding | 3.40E−11 | 3.824954089 |
| A730011C13Rik | lncRNA | 3.77E−11 | 2.734827962 |
| Gm49673 | protein coding | 4.36E−11 | 3.667821853 |
| Apol7c | protein coding | 5.60E−11 | 3.868314379 |
| Gm15998 | lncRNA | 5.97E−11 | 5.569039579 |
| Dnmt3c | protein coding | 1.35E−10 | 2.524769715 |
| Tgtp1 | protein coding | 1.91E−10 | 5.717746564 |
| 4933432I03Rik | lncRNA | 3.22E−10 | 2.917899504 |
| Gm12764 | lncRNA | 3.75E−10 | 2.782842147 |
| AW112010 | lncRNA | 3.76E−10 | 2.77729885 |
| Arhgap28 | protein coding | 3.92E−10 | 2.341310179 |
| Gm8773 | protein coding | 5.33E−10 | 2.884399291 |
| Cmpk2 | protein coding | 6.54E−10 | 3.231946893 |
| Gstt1 | protein coding | 6.67E−10 | 2.542479307 |
| Gm14569 | protein coding | 8.60E−10 | 4.392408877 |
| Fam3b | protein coding | 1.17E−09 | 3.498587656 |
| Lad1 | protein coding | 1.44E−09 | 2.533598273 |
| Phf11a | protein coding | 1.98E−09 | 2.548076204 |
| Gm12551 | unprocessed pseudogene | 2.07E−09 | 2.909574841 |
| Apod | protein coding | 2.22E−09 | 2.711558715 |
| Ifnab | protein coding | 2.48E−09 | 5.608806463 |
| Lpar1 | protein coding | 2.97E−09 | 2.535910208 |
| Ifna14 | protein coding | 3.86E−09 | 7.161073042 |
| Ifna12 | protein coding | 3.91E−09 | 8.781089892 |
| Gm14199 | lncRNA | 4.19E−09 | 5.034931526 |
| Gm46189 | lncRNA | 4.35E−09 | 3.249912097 |
| A930015D03Rik | lncRNA | 5.99E−09 | 3.219735965 |
| Trim30c | protein coding | 1.22E−08 | 2.462893763 |
| Cd69 | protein coding | 1.25E−08 | 3.472006051 |
| Gm45193 | lncRNA | 1.54E−08 | 2.360244874 |
| Tnfsf9 | protein coding | 1.59E−08 | 2.333443931 |
| Cxcl10 | protein coding | 1.63E−08 | 4.008256833 |
| Gm15056 | protein coding | 1.80E−08 | 8.318440223 |
| Gbp2b | protein coding | 2.58E−08 | 3.598679854 |
| Rsad2 | protein coding | 3.07E−08 | 2.859032153 |
| Ifna15 | protein coding | 3.15E−08 | 6.815658422 |
| Gm49662 | lncRNA | 5.68E−08 | 3.980943952 |
| Ifit1bl1 | protein coding | 6.88E−08 | 3.585644536 |
| Ifit2 | protein coding | 7.73E−08 | 2.340973443 |
| Gm37711 | TEC | 8.10E−08 | 3.575291007 |
| Gnb1-ps3 | processed pseudogene | 8.24E−08 | 4.101842898 |
| Klri1 | protein coding | 8.34E−08 | 4.217587043 |
| Coch | protein coding | 8.97E−08 | 6.685300815 |
| Ccr7 | protein coding | 1.08E−07 | 2.534385002 |
| Dll1 | protein coding | 1.52E−07 | 2.332778855 |
| Calhm6 | protein coding | 2.02E−07 | 3.359621088 |
| Gm12840 | lncRNA | 2.44E−07 | 2.802393239 |
| Ptgs2os2 | lncRNA | 2.55E−07 | 2.344620795 |
| Kpna2-ps | processed pseudogene | 2.67E−07 | 2.687232181 |
| Gm8818 | processed pseudogene | 3.01E−07 | 6.338403523 |
| Gpr31b | protein coding | 3.23E−07 | 3.104005429 |
| Gbp10 | protein coding | 3.26E−07 | 3.375495713 |
| Gbp4 | protein coding | 5.60E−07 | 2.815883751 |
| Gm12185 | protein coding | 5.72E−07 | 3.107903631 |
| Gm8221 | unprocessed pseudogene | 7.33E−07 | 5.325949068 |
| Iigp1 | protein coding | 7.49E−07 | 3.388237482 |
| Gm19076 | processed pseudogene | 8.68E−07 | 2.407544922 |
| Gm7281 | unprocessed pseudogene | 8.98E−07 | 3.894228032 |
| Gm13391 | lncRNA | 1.11E−06 | 2.434902463 |
| Gm13713 | lncRNA | 1.60E−06 | 4.440235346 |
| BE692007 | lncRNA | 1.73E−06 | 2.371876467 |
| Gm21748 | protein coding | 2.64E−06 | 2.992552638 |
| Tagap | protein coding | 3.36E−06 | 2.374359242 |
| Gm18445 | unprocessed pseudogene | 3.62E−06 | 3.44243698 |
| G530011O06Rik | lncRNA | 6.49E−06 | 2.748833068 |
| Ifna16 | protein coding | 8.09E−06 | 5.646069405 |
| 4930445E18Rik | lncRNA | 1.91E−05 | 3.964819287 |
| Gm4761 | processed pseudogene | 2.26E−05 | 3.520021162 |
| Prm1 | protein coding | 2.52E−05 | 3.870405568 |
| Gm10552 | lncRNA | 2.56E−05 | 2.423192424 |
| Tgtp2 | protein coding | 2.74E−05 | 2.731545968 |
| Gm15265 | lncRNA | 3.00E−05 | 2.440858558 |
| Gm28347 | lncRNA | 3.64E−05 | 5.375585669 |
| Hsf2bp | protein coding | 4.25E−05 | 2.961277385 |
| Gm15754 | transcribed processed | 5.39E−05 | 2.594163863 |
| | pseudogene | | |
| Xcl1 | protein coding | 8.43E−05 | 4.664867009 |
| Gm28809 | lncRNA | 8.87E−05 | 2.508926293 |
| Gm18342 | unprocessed pseudogene | 9.70E−05 | 2.66502253 |
| Gm44135 | TEC | 0.000178264 | 3.890664248 |
| AC167036.2 | protein coding | 0.000204159 | 2.583238457 |
| Bmp10 | protein coding | 0.000251487 | 3.425325521 |
| Gm2619 | lncRNA | 0.000367401 | 5.972608171 |
| Gm49356 | protein coding | 0.000439989 | 2.402391818 |
| Aplnr | protein coding | 0.000566532 | 2.346333776 |

Fold change is quantified in fragments per kilobase of transcript per million mapped reads (FKBP) and displayed as the mean log-ratio of gene expression in cells treated with eccDNA versus cells treated with linear DNA (n = 3).

TABLE 2
Differentially expressed genes in BMDMs treated with eccDNA

| Gene: | Gene type: | Adjusted p-value: | Log₂ fold change: |
|---|---|---|---|
| Cd274 | protein coding | 0 | 3.791531734 |
| Cmpk2 | protein coding | 0 | 5.333625512 |
| Rsad2 | protein coding | 0 | 5.333358429 |
| Wars | protein coding | 0 | 2.502666402 |
| C3 | protein coding | 0 | 4.488690165 |
| Usp18 | protein coding | 0 | 3.658760179 |
| Cd69 | protein coding | 0 | 6.781962383 |
| H2-Q4 | protein coding | 0 | 2.62642828 |
| Tnfsf10 | protein coding | 0 | 5.462279887 |
| Fgl2 | protein coding | 0 | 5.377266377 |
| Oasl1 | protein coding | 0 | 4.765625917 |
| Calhm6 | protein coding | 0 | 4.706422018 |
| Iigp1 | protein coding | 0 | 5.977981308 |
| Kdr | protein coding | 0 | 4.949852371 |
| Apol9b | protein coding | 0 | 5.483859198 |
| Pou3f1 | protein coding | 0 | 4.374794774 |
| 1600014C10Rik | protein coding | 5.06E−292 | 3.176220181 |
| Csf1 | protein coding | 6.84E−292 | 4.075449176 |
| Ccrl2 | protein coding | 1.43E−290 | 3.747859887 |
| Jak2 | protein coding | 2.33E−288 | 2.741584706 |
| Usb1 | protein coding | 2.76E−288 | 2.596024811 |
| Il1rn | protein coding | 5.22E−270 | 4.942710775 |
| Nod1 | protein coding | 1.85E−263 | 3.310282006 |
| Gbp6 | protein coding | 2.15E−262 | 4.748705 |
| Ifit3b | protein coding | 2.85E−260 | 4.518877301 |
| Cd86 | protein coding | 1.24E−259 | 4.231635154 |
| Rnf34 | protein coding | 2.27E−257 | 2.671319848 |
| Fndc3a | protein coding | 5.13E−257 | 2.56047363 |
| Il18bp | protein coding | 1.88E−256 | 4.119423573 |
| Aldh1b1 | protein coding | 8.45E−254 | 3.410949755 |
| Ifi208 | protein coding | 2.91E−251 | 4.128961118 |
| Tap1 | protein coding | 2.43E−250 | 2.738000773 |
| Daxx | protein coding | 1.31E−248 | 3.224004888 |
| Slc28a2 | protein coding | 8.91E−242 | 3.639521445 |
| Parp10 | protein coding | 1.03E−240 | 2.669998657 |
| A530040E14Rik | lncRNA | 7.32E−240 | 5.142545124 |
| Igtp | protein coding | 2.02E−239 | 3.298613458 |
| Mtus1 | protein coding | 6.40E−238 | 3.325720598 |
| Parp3 | protein coding | 1.64E−237 | 2.740975734 |
| Batf2 | protein coding | 2.36E−236 | 3.723603021 |
| Slc25a22 | protein coding | 2.08E−235 | 3.132412643 |
| Slc2a6 | protein coding | 2.30E−233 | 3.272286763 |
| H2-Q6 | protein coding | 8.17E−227 | 3.740838045 |
| Ifit3 | protein coding | 1.94E−226 | 3.657091013 |
| AA467197 | protein coding | 7.37E−224 | 6.18996897 |
| Xaf1 | protein coding | 1.08E−222 | 2.44629127 |
| Arel1 | protein coding | 3.76E−222 | 2.469755916 |
| Setdb2 | protein coding | 3.82E−218 | 2.871262399 |
| Irf1 | protein coding | 8.83E−218 | 2.645360443 |
| Ccnd1 | protein coding | 1.21E−217 | 2.584840391 |
| Isg15 | protein coding | 1.75E−216 | 3.943600514 |
| Psmb9 | protein coding | 3.94E−214 | 2.364417593 |
| Ifit1 | protein coding | 1.51E−212 | 3.501011511 |
| Plaat3 | protein coding | 3.30E−211 | 3.630581758 |
| Irgm2 | protein coding | 2.69E−209 | 2.668332285 |
| Il15 | protein coding | 1.97E−208 | 3.262784038 |
| Ccnd2 | protein coding | 4.98E−205 | 3.783935293 |
| Rapgef2 | protein coding | 3.13E−204 | 3.322344504 |
| Sp140 | protein coding | 1.45E−203 | 2.993133376 |
| Gca | protein coding | 1.19E−202 | 4.417157184 |
| Tlr9 | protein coding | 2.46E−199 | 3.034181759 |
| Themis2 | protein coding | 4.28E−199 | 2.467754062 |
| Slamf9 | protein coding | 1.34E−193 | 2.354409356 |
| Acsl1 | protein coding | 4.60E−192 | 3.311558125 |
| Phf11a | protein coding | 3.03E−191 | 4.569847517 |
| Tor3a | protein coding | 4.27E−191 | 2.655504339 |
| Irf7 | protein coding | 1.15E−188 | 3.200328547 |
| Kif5c | protein coding | 4.84E−186 | 3.689861655 |
| Mthfr | protein coding | 8.31E−186 | 2.702320996 |
| Gbp8 | protein coding | 9.63E−186 | 3.362817862 |
| Gm12250 | processed pseudogene | 4.43E−185 | 2.945074044 |
| Gch1 | protein coding | 8.01E−185 | 2.387101138 |
| Enpp4 | protein coding | 1.57E−184 | 4.150278608 |
| Slco3a1 | protein coding | 2.51E−184 | 4.41497407 |
| Psmb10 | protein coding | 9.76E−183 | 2.676715143 |
| Tlr3 | protein coding | 1.14E−176 | 2.990579316 |
| Mxd1 | protein coding | 1.53E−173 | 3.204794812 |
| Trim30c | protein coding | 2.38E−173 | 4.326186486 |
| Psme2 | protein coding | 1.84E−172 | 2.352649539 |
| Ifih1 | protein coding | 7.02E−172 | 3.008542694 |
| Gm12185 | protein coding | 1.24E−167 | 3.746189646 |
| Rilpl1 | protein coding | 5.70E−167 | 2.90524874 |
| C130026I21Rik | protein coding | 2.08E−165 | 3.610490523 |
| Fcgr1 | protein coding | 1.45E−164 | 2.848084644 |
| Mlkl | protein coding | 7.60E−163 | 2.932595924 |
| Ifi214 | protein coding | 4.42E−162 | 4.068884396 |
| Peli1 | protein coding | 7.00E−162 | 2.60602298 |
| Abcb1a | protein coding | 7.75E−162 | 3.442507609 |
| H2-T22 | protein coding | 1.85E−160 | 2.51225309 |
| Slfn9 | protein coding | 3.67E−160 | 3.12584053 |
| Il21r | protein coding | 2.21E−159 | 2.411006609 |
| 4930430E12Rik | lncRNA | 3.69E−159 | 2.438489187 |
| Socs1 | protein coding | 4.18E−158 | 4.208719317 |
| Gm4951 | protein coding | 5.88E−158 | 3.334114634 |
| Kcnab1 | protein coding | 8.94E−158 | 4.318442896 |
| Parp11 | protein coding | 9.28E−158 | 2.968793473 |
| March5 | protein coding | 1.61E−157 | 2.397496039 |
| Arhgef3 | protein coding | 3.06E−157 | 2.448105119 |
| Marcksl1 | protein coding | 4.73E−157 | 3.490454747 |
| Phf11c | protein coding | 1.43E−156 | 3.82378112 |
| Ripk2 | protein coding | 6.38E−156 | 2.482013816 |
| Slfn1 | protein coding | 4.30E−155 | 3.276111536 |
| Klrk1 | protein coding | 4.46E−155 | 4.479052775 |
| Ifi47 | protein coding | 1.60E−153 | 2.832273334 |
| Selenow | protein coding | 1.67E−152 | 2.419253712 |
| BC147527 | protein coding | 2.63E−151 | 3.654913044 |
| Asb13 | protein coding | 3.43E−151 | 2.565202249 |
| Helz2 | protein coding | 3.80E−151 | 2.629816859 |
| Tent5c | protein coding | 9.62E−151 | 2.665599844 |
| Mov10 | protein coding | 2.45E−150 | 2.505287394 |
| Apobec3 | protein coding | 3.28E−150 | 2.338014722 |
| Nt5c3 | protein coding | 3.90E−148 | 2.83655331 |
| Casp4 | protein coding | 3.17E−145 | 2.854527199 |
| Ly6a | protein coding | 5.37E−145 | 2.648023135 |
| Trim30b | protein coding | 9.11E−142 | 3.670376888 |
| Ddx60 | protein coding | 3.60E−139 | 3.115950947 |
| Mmp25 | protein coding | 5.32E−139 | 5.296945886 |
| Stat2 | protein coding | 3.68E−138 | 2.570433453 |
| Cxcl10 | protein coding | 5.76E−138 | 7.347041103 |
| Ddx58 | protein coding | 1.34E−137 | 2.453385717 |
| Pnp | protein coding | 5.10E−137 | 2.580794383 |
| Ifi206 | protein coding | 6.65E−137 | 3.178597881 |
| Il15ra | protein coding | 2.43E−136 | 3.960853569 |
| Arid5a | protein coding | 4.12E−136 | 3.261742026 |
| Igsf9 | protein coding | 1.57E−133 | 3.991835114 |
| Ccl5 | protein coding | 5.55E−133 | 7.617097893 |
| Tmem67 | protein coding | 9.94E−133 | 2.651477932 |
| Slamf8 | protein coding | 4.42E−131 | 3.462362409 |
| Apol9a | protein coding | 4.48E−131 | 6.702980822 |
| Gbp4 | protein coding | 1.27E−130 | 6.87306418 |
| Bcl2a1d | protein coding | 1.96E−128 | 3.601597892 |
| Mx2 | polymorphic pseudogene | 3.59E−128 | 3.694872015 |
| Icam1 | protein coding | 1.09E−126 | 2.699304379 |
| Abtb2 | protein coding | 2.97E−126 | 5.347421562 |
| Gbp5 | protein coding | 8.57E−126 | 5.637204745 |
| Tnfsf15 | protein coding | 2.75E−125 | 5.018449284 |
| Ppm1k | protein coding | 5.62E−125 | 2.342131891 |
| Flt1 | protein coding | 5.60E−124 | 4.11682831 |
| Ccl4 | protein coding | 2.94E−123 | 2.841740914 |
| Oas1b | polymorphic pseudogene | 5.36E−121 | 2.756633674 |
| Atp10a | protein coding | 1.25E−118 | 2.576654281 |
| Ifi211 | protein coding | 5.82E−118 | 2.709328409 |
| Slc7a8 | protein coding | 3.87E−117 | 2.584470181 |
| Tnfsf8 | protein coding | 9.17E−117 | 4.411289129 |
| Mfsd7a | protein coding | 9.81E−117 | 2.572921233 |
| Gnb4 | protein coding | 1.27E−116 | 2.535014409 |
| Cfb | protein coding | 1.46E−114 | 6.361505739 |
| Phf11d | protein coding | 1.81E−114 | 3.362567055 |
| Ctla2b | protein coding | 2.68E−114 | 2.514698411 |
| Tapbpl | protein coding | 1.80E−113 | 2.82499696 |
| Tmem171 | protein coding | 2.55E−113 | 3.038846854 |
| Cd200r4 | protein coding | 3.98E−113 | 2.459160612 |
| I830077J02Rik | protein coding | 1.17E−112 | 3.121745115 |
| Gbp9 | protein coding | 6.71E−111 | 2.692568619 |
| Ifi203-ps | unprocessed pseudogene | 1.29E−110 | 2.716412303 |
| Gm49342 | protein coding | 4.21E−110 | 2.538876505 |
| Glipr2 | protein coding | 4.61E−110 | 4.372098798 |
| Lipg | protein coding | 3.18E−108 | 8.657784262 |
| Fcgr4 | protein coding | 1.26E−105 | 2.985564272 |
| Zup1 | protein coding | 5.37E−104 | 2.40586493 |
| Gm21860 | lncRNA | 4.49E−103 | 3.173183438 |
| Herc6 | protein coding | 6.35E−102 | 3.006725992 |
| Lhx2 | protein coding | 1.20E−100 | 5.998848144 |
| Traf1 | protein coding | 4.63E−100 | 2.898642499 |
| Igf2bp2 | protein coding | 7.97E−100 | 2.362820925 |
| Ass1 | protein coding | 9.01E−100 | 2.623406738 |
| Mitd1 | protein coding | 9.51E−100 | 2.329195703 |
| Dnase1l3 | protein coding | 1.38E−98 | 6.915033957 |
| Creb5 | protein coding | 2.78E−98 | 2.333083533 |
| Rgl1 | protein coding | 1.19E−97 | 2.886559045 |
| H2-Q7 | protein coding | 1.77E−97 | 3.056712946 |
| Ifit1bl1 | protein coding | 4.13E−97 | 7.078188807 |
| Stx11 | protein coding | 4.64E−97 | 2.553634374 |
| Timeless | protein coding | 7.11E−97 | 2.747913268 |
| Gm42547 | TEC | 3.88E−95 | 2.713054485 |
| Ifi209 | protein coding | 1.19E−94 | 2.670974009 |
| 4933412E12Rik | lncRNA | 1.88E−94 | 3.193856886 |
| Ifi213 | protein coding | 3.92E−94 | 2.885610139 |
| Il27 | protein coding | 2.61E−93 | 5.310743395 |
| Slfn4 | protein coding | 3.32E−93 | 5.116218161 |
| Sp110 | protein coding | 4.67E−92 | 2.454011773 |
| Dusp28 | protein coding | 7.04E−92 | 2.886658091 |
| Ctsc | protein coding | 5.94E−91 | 2.423677687 |
| Irgm1 | protein coding | 1.02E−89 | 2.350311464 |
| Mill2 | protein coding | 5.56E−86 | 2.375712565 |
| Isg20 | protein coding | 3.68E−84 | 5.091668023 |
| Nox1 | protein coding | 4.08E−84 | 3.114069082 |
| Cd300lf | protein coding | 7.17E−83 | 2.770271377 |
| Mx1 | polymorphic pseudogene | 1.94E−82 | 4.619967895 |
| Ifi205 | protein coding | 7.84E−82 | 6.236406012 |
| AW112010 | lncRNA | 3.14E−81 | 5.250397723 |
| Cp | protein coding | 5.33E−81 | 2.434371096 |
| Fanca | protein coding | 1.91E−80 | 2.929883842 |
| Vcan | protein coding | 2.69E−80 | 4.135799598 |
| Ddx4 | protein coding | 8.63E−80 | 4.208564552 |
| AC147806.2 | protein coding | 4.27E−76 | 4.535048547 |
| AW011738 | lncRNA | 4.65E−76 | 2.951858998 |
| Nmi | protein coding | 4.58E−75 | 2.384742293 |
| Hap1 | protein coding | 1.57E−74 | 3.571383678 |
| Tnf | protein coding | 3.69E−74 | 2.372771261 |
| Acod1 | protein coding | 4.07E−73 | 6.937045188 |
| Cd40 | protein coding | 4.42E−73 | 4.745693619 |
| Ccl2 | protein coding | 8.86E−72 | 4.21664569 |
| Ifit1bl2 | protein coding | 9.38E−70 | 3.043139798 |
| Sco1 | protein coding | 1.09E−69 | 2.350535957 |
| 9330175E14Rik | lncRNA | 1.22E−69 | 2.333327544 |
| Il13ra1 | protein coding | 2.12E−67 | 2.809342854 |
| Pnp2 | protein coding | 5.52E−67 | 2.829107247 |
| 9530082P21Rik | lncRNA | 3.30E−66 | 2.408546533 |
| Slc43a3 | protein coding | 5.94E−65 | 2.36764155 |
| Gm1966 | unprocessed pseudogene | 4.58E−64 | 3.193646161 |
| Treml2 | protein coding | 1.65E−61 | 4.959890001 |
| A530032D15Rik | protein coding | 1.97E−61 | 3.499486693 |
| Sh2d6 | protein coding | 2.22E−61 | 3.558267629 |
| Serpina3f | protein coding | 3.84E−61 | 6.763145007 |
| Cxcl9 | protein coding | 4.05E−61 | 7.603723514 |
| Hsh2d | protein coding | 5.78E−61 | 5.05047843 |
| Gbp7 | protein coding | 2.63E−59 | 3.938585883 |
| Gbp3 | protein coding | 4.91E−59 | 4.125038246 |
| Ifit2 | protein coding | 5.15E−58 | 3.775451587 |
| Misp | protein coding | 1.02E−57 | 3.810617275 |
| Plagl1 | protein coding | 1.65E−57 | 4.768885426 |
| Tgtp2 | protein coding | 2.04E−57 | 4.245293243 |
| Il10 | protein coding | 8.32E−57 | 2.997101676 |
| Soat2 | protein coding | 9.28E−57 | 2.325354379 |
| Fabp3 | protein coding | 1.28E−55 | 2.620392091 |
| Ssc5d | protein coding | 2.25E−55 | 2.705921374 |
| Glrp1 | protein coding | 2.47E−55 | 2.978314986 |
| Ccl12 | protein coding | 6.32E−55 | 6.63830705 |
| A330040F15Rik | lncRNA | 3.00E−54 | 5.267874027 |
| Sectm1a | protein coding | 4.97E−54 | 6.720973524 |
| Gm6545 | processed pseudogene | 7.07E−54 | 4.717878706 |
| Klra2 | protein coding | 9.46E−54 | 2.667965952 |
| Flt4 | protein coding | 6.71E−53 | 5.359408751 |
| Olfr56 | protein coding | 4.48E−52 | 4.834691657 |
| Phf11b | protein coding | 3.42E−51 | 3.705330218 |
| Trp53i11 | protein coding | 3.55E−51 | 5.326178515 |
| Ccdc173 | protein coding | 1.00E−49 | 3.166733689 |
| Gm47242 | lncRNA | 1.39E−49 | 5.154684948 |
| Ch25h | protein coding | 7.69E−49 | 3.890134538 |
| Scimp | protein coding | 3.92E−48 | 5.232530359 |
| Socs3 | protein coding | 1.05E−47 | 2.694699525 |
| Dennd6b | protein coding | 1.55E−47 | 2.604934599 |
| Cst7 | protein coding | 1.69E−47 | 5.219482815 |
| Ifitm6 | protein coding | 2.24E−46 | 2.685640845 |
| Ifi44 | protein coding | 6.27E−46 | 3.482859724 |
| A630012P03Rik | lncRNA | 4.17E−45 | 5.614437441 |
| Gypc | protein coding | 2.29E−44 | 2.500946574 |
| Map2 | protein coding | 3.33E−44 | 2.636245215 |
| Gpr55 | protein coding | 7.08E−44 | 5.306884563 |
| Tgtp1 | protein coding | 1.07E−42 | 7.833626118 |
| A330074K22Rik | lncRNA | 2.62E−41 | 3.100542929 |
| Gm16675 | lncRNA | 5.07E−41 | 2.644784952 |
| Ntn1 | protein coding | 1.38E−40 | 3.959304932 |
| Nos2 | protein coding | 9.70E−40 | 9.891658158 |
| Gbp2 | protein coding | 2.65E−37 | 4.785052589 |
| Gm18853 | unprocessed pseudogene | 2.88E−37 | 2.89320159 |
| Gm18852 | processed pseudogene | 3.74E−37 | 2.623016087 |
| Smpdl3b | protein coding | 1.08E−36 | 2.658237572 |
| Bcl2a1a | protein coding | 1.50E−36 | 4.351132325 |
| Chst15 | protein coding | 4.15E−36 | 2.535307433 |
| Gm10553 | lncRNA | 4.17E−36 | 5.238938754 |
| Ms4a4c | protein coding | 1.08E−35 | 4.949722126 |
| Zbp1 | protein coding | 5.49E−35 | 2.925389502 |
| Pla2g4c | protein coding | 3.11E−34 | 4.693865395 |
| Cxcl11 | polymorphic pseudogene | 9.06E−34 | 12.59589588 |
| Kmo | protein coding | 1.44E−33 | 3.774816016 |
| St3gal6 | protein coding | 2.21E−33 | 2.799328552 |
| Phf11 | unprocessed pseudogene | 3.11E−33 | 3.172010143 |
| Gm5970 | processed pseudogene | 5.43E−33 | 3.231268483 |
| Ccl7 | protein coding | 1.07E−31 | 3.997980131 |
| Gm5424 | processed pseudogene | 1.08E−30 | 2.795236819 |
| Gm10552 | lncRNA | 7.43E−30 | 4.122430843 |
| BC023105 | processed pseudogene | 1.55E−29 | 5.869930776 |
| A730011C13Rik | lncRNA | 1.84E−29 | 3.450344449 |
| Gm7609 | protein coding | 2.04E−29 | 4.320360083 |
| Gpr84 | protein coding | 2.64E−29 | 2.654485996 |
| Serpina3g | protein coding | 2.09E−28 | 7.257765848 |
| Cysltr2 | protein coding | 2.50E−28 | 2.656315523 |
| Dmtn | protein coding | 3.70E−28 | 3.915620558 |
| Clcn1 | protein coding | 1.21E−26 | 3.115840661 |
| Slc7a2 | protein coding | 3.71E−25 | 2.393295571 |
| Gm19026 | processed pseudogene | 6.08E−25 | 3.236196166 |
| C4b | protein coding | 1.19E−24 | 4.978988904 |
| Ifnb1 | protein coding | 2.33E−24 | 5.86161849 |
| Hrh2 | protein coding | 2.50E−24 | 3.449803173 |
| Angpt1 | protein coding | 3.47E−24 | 5.352417267 |
| Apod | protein coding | 2.14E−23 | 4.118646111 |
| Ccl8 | protein coding | 1.35E−22 | 4.941657967 |
| Gm2065 | TEC | 3.77E−22 | 5.226856868 |
| H2-M2 | protein coding | 6.25E−22 | 2.884925813 |
| Itpr1 | protein coding | 1.70E−21 | 2.473241068 |
| Tlr11 | protein coding | 1.94E−20 | 3.264265115 |
| Gbp11 | polymorphic pseudogene | 4.04E−20 | 7.921764302 |
| Tnfsf4 | protein coding | 4.34E−20 | 3.601712775 |
| Clic5 | protein coding | 2.11E−19 | 5.848734144 |
| Stk39 | protein coding | 4.57E−19 | 3.055969175 |
| Cadps | protein coding | 2.43E−18 | 3.400599579 |
| AC167036.1 | protein coding | 7.89E−18 | 3.892671964 |
| Rhou | protein coding | 2.38E−17 | 2.426897817 |
| Gm34643 | lncRNA | 2.47E−17 | 4.131318153 |
| Gm19684 | protein coding | 2.90E−17 | 3.166780988 |
| Gpr31b | protein coding | 3.28E−17 | 2.525757259 |
| Il12rb1 | protein coding | 3.59E−17 | 6.007094557 |
| Heatr9 | protein coding | 5.08E−17 | 10.02828504 |
| Dll1 | protein coding | 5.83E−17 | 4.553471271 |
| Gm16094 | lncRNA | 1.06E−16 | 4.341780083 |
| Gm45418 | lncRNA | 2.19E−16 | 2.624629316 |
| Gm35551 | lncRNA | 2.44E−16 | 5.473832328 |
| Piwil4 | protein coding | 2.64E−16 | 3.124993911 |
| Kynu | protein coding | 3.12E−16 | 3.263302436 |
| Socs2 | protein coding | 3.59E−16 | 3.065736712 |
| Rgs16 | protein coding | 3.84E−16 | 2.686297379 |
| Majin | protein coding | 4.25E−16 | 3.789235348 |
| Ankmy1 | protein coding | 1.59E−15 | 2.656855264 |
| Gm43814 | TEC | 2.81E−15 | 3.731380315 |
| Il6 | protein coding | 1.55E−14 | 9.305554395 |
| Gm14569 | protein coding | 2.61E−14 | 8.781118921 |
| Dnmt3c | protein coding | 3.08E−14 | 6.007511131 |
| Mir155hg | lncRNA | 3.61E−14 | 6.209401016 |
| Col9a3 | protein coding | 4.58E−14 | 2.498506355 |
| Gm15753 | unprocessed pseudogene | 1.09E−13 | 2.936619692 |
| Noxred1 | protein coding | 1.49E−13 | 2.937402995 |
| Slc6a4 | protein coding | 1.58E−13 | 4.180491266 |
| Lpar1 | protein coding | 1.63E−13 | 2.934390345 |
| Upp1 | protein coding | 1.76E−13 | 4.084184448 |
| Gm12764 | lncRNA | 4.72E−13 | 2.901434582 |
| Plekha4 | protein coding | 5.59E−13 | 2.616994541 |
| Cnn3 | protein coding | 1.15E−12 | 3.118370008 |
| Spats2l | protein coding | 3.17E−12 | 5.076095537 |
| Hdc | protein coding | 3.36E−12 | 5.150680284 |
| Mid1 | protein coding | 3.45E−12 | 2.858116682 |
| Gm45193 | lncRNA | 4.03E−12 | 9.692077112 |
| Tspan33 | protein coding | 4.39E−12 | 2.486380338 |
| Gm4841 | protein coding | 4.77E−12 | 7.352782048 |
| Pla2r1 | protein coding | 7.04E−12 | 2.768689942 |
| Saa3 | protein coding | 1.41E−11 | 3.08958282 |
| Gm49730 | TEC | 4.68E−11 | 2.9323199 |
| Slc39a2 | protein coding | 6.39E−11 | 2.341878732 |
| Gm12474 | lncRNA | 6.63E−11 | 3.992921132 |
| Fbn1 | protein coding | 7.28E−11 | 3.313453545 |
| F830016B08Rik | protein coding | 8.52E−11 | 2.371971585 |
| Clec9a | protein coding | 8.81E−11 | 2.876251285 |
| Gm4117 | lncRNA | 1.04E−10 | 3.306086663 |
| Pgf | protein coding | 6.46E−10 | 2.809763394 |
| G530011O06Rik | lncRNA | 8.48E−10 | 3.809107559 |
| 9330179D12Rik | lncRNA | 1.12E−09 | 3.360893728 |
| Gm12216 | protein coding | 1.56E−09 | 2.536946369 |
| AC147806.1 | protein coding | 2.45E−09 | 8.625442392 |
| Gm13822 | lncRNA | 2.77E−09 | 3.300760665 |
| Clec4e | protein coding | 6.67E−09 | 3.049854581 |
| Gm19076 | processed pseudogene | 7.33E−09 | 2.557572063 |
| Gpr31a | processed pseudogene | 9.52E−09 | 3.465685218 |
| 4930471E19Rik | lncRNA | 1.36E−08 | 2.964677101 |
| Gm49662 | lncRNA | 3.08E−08 | 5.21551598 |
| Gm614 | protein coding | 3.76E−08 | 3.512929861 |
| AC167036.2 | protein coding | 8.57E−08 | 3.385798131 |
| Ly6i | protein coding | 1.03E−07 | 2.965093644 |
| Tarm1 | protein coding | 1.17E−07 | 2.441300831 |
| Mid1-ps1 | unprocessed pseudogene | 1.18E−07 | 2.929429494 |
| Art3 | protein coding | 1.32E−07 | 3.240786404 |
| 1190001M18Rik | lncRNA | 2.14E−07 | 2.44237051 |
| Ly6c2 | protein coding | 3.38E−07 | 2.674672939 |
| Fam3b | protein coding | 3.77E−07 | 6.332952134 |
| Ubd | protein coding | 4.58E−07 | 5.581579041 |
| Gm21748 | protein coding | 8.29E−07 | 3.216872624 |
| Gm14199 | lncRNA | 1.24E−06 | 6.103822738 |
| Gm18752 | transcribed unprocessed | 1.49E−06 | 3.241158941 |
| | pseudogene | | |
| Gm49356 | protein coding | 1.92E−06 | 2.853293585 |
| Gm18342 | unprocessed pseudogene | 3.50E−06 | 5.197883778 |
| Prm1 | protein coding | 4.66E−06 | 7.002149512 |
| Gm8463 | processed pseudogene | 8.78E−06 | 4.003242619 |
| Gm15754 | transcribed processed | 1.16E−05 | 6.70022183 |
| | pseudogene | | |
| Gm20627 | lncRNA | 1.76E−05 | 2.84975903 |
| Gm28347 | lncRNA | 1.76E−05 | 6.733888255 |
| Gm12551 | unprocessed pseudogene | 3.55E−05 | 3.247074581 |
| AC123856.1 | transcribed unprocessed | 4.90E−05 | 5.267683994 |
| | pseudogene | | |
| Gm49392 | protein coding | 6.51E−05 | 4.076687017 |
| Gm21742 | unprocessed pseudogene | 0.000438035 | 3.230473535 |
| Cdhr4 | protein coding | 0.000792976 | 2.599961461 |

Fold change is quantified in fragments per kilobase of transcript per million mapped reads (FKBP) and displayed as the mean log-ratio of gene expression in cells treated with eccDNA versus cells treated with linear DNA (n = 3).

TABLE 3
Differentially expressed genes in BMDCs in response to STING or Myd88 knockout with eccDNA treatment

| | | | | STING KO + | Myd88 KO + |
|---|---|---|---|---|---|
| Gene: | Gene type: | WT + mock: | WT + eccDNA | eccDNA | eccDNA |
| Acsl1 | protein coding | 33.97 ± 1.45 | 434.49 ± 18.81 | 25.06 ± 1.05 | 361.18 ± 10.00 |
| Nos2 | protein coding | 1.29 ± 0.17 | 237.24 ± 27.30 | 0.98 ± 0.01 | 160.14 ± 10.59 |
| Acod1 | protein coding | 101.60 ± 6.34 | 1717.17 ± 62.51 | 31.14 ± 8.22 | 1475.54 ± 3.66 |
| Flnb | protein coding | 4.42 ± 0.19 | 28.20 ± 0.86 | 4.53 ± 0.17 | 23.12 ± 0.18 |
| Dnase1l3 | protein coding | 0.54 ± 0.13 | 35.34 ± 4.61 | 0.40 ± 0.21 | 44.61 ± 1.52 |
| Ccl5 | protein coding | 74.12 ± 16.33 | 4131.16 ± 460.39 | 102.18 ± 11.02 | 4412.30 ± 192.18 |
| Serpinb9 | protein coding | 21.70 ± 1.49 | 225.65 ± 24.07 | 24.15 ± 0.91 | 246.62 ± 9.07 |
| Tnf | protein coding | 13.61 ± 0.69 | 103.78 ± 2.76 | 6.27 ± 0.52 | 69.31 ± 5.88 |
| Clic4 | protein coding | 171.66 ± 1.58 | 654.04 ± 13.76 | 139.43 ± 7.49 | 613.16 ± 7.11 |
| Serpina3f | protein coding | 1.20 ± 0.32 | 46.92 ± 2.11 | 0.18 ± 0.06 | 30.90 ± 0.45 |
| Tnfaip3 | protein coding | 17.03 ± 0.52 | 86.27 ± 2.88 | 12.84 ± 0.01 | 73.57 ± 0.71 |
| Clec4e | protein coding | 19.98 ± 3.02 | 548.78 ± 111.70 | 25.23 ± 0.33 | 494.02 ± 27.61 |
| Plaat3 | protein coding | 7.89 ± 0.68 | 67.73 ± 3.16 | 8.73 ± 0.98 | 80.74 ± 0.30 |
| AA467197 | protein coding | 265.21 ± 18.78 | 1689.33 ± 126.18 | 284.74 ± 22.54 | 1849.34 ± 7.37 |
| Slco3a1 | protein coding | 5.02 ± 0.43 | 45.42 ± 3.53 | 3.92 ± 0.11 | 40.49 ± 1.85 |
| Inhba | protein coding | 27.02 ± 4.74 | 313.73 ± 27.29 | 18.38 ± 0.01 | 217.05 ± 1.31 |
| Tpx2 | protein coding | 10.57 ± 0.72 | 55.54 ± 0.99 | 7.55 ± 0.14 | 55.56 ± 1.97 |
| Slc7a2 | protein coding | 32.09 ± 3.95 | 188.98 ± 3.58 | 23.82 ± 1.16 | 117.99 ± 1.64 |
| Tgm2 | protein coding | 19.45 ± 0.82 | 139.72 ± 16.41 | 22.11 ± 0.18 | 130.53 ± 6.02 |
| Tnfsf15 | protein coding | 10.71 ± 1.03 | 66.82 ± 4.64 | 6.87 ± 0.14 | 43.65 ± 0.34 |
| Rapgef2 | protein coding | 7.64 ± 0.66 | 57.55 ± 5.39 | 5.46 ± 0.17 | 37.69 ± 0.95 |
| Tma16 | protein coding | 18.94 ± 2.40 | 106.24 ± 2.19 | 19.19 ± 0.81 | 137.81 ± 3.61 |
| Apol9b | protein coding | 3.78 ± 0.91 | 58.26 ± 0.62 | 0.92 ± 0.83 | 65.59 ± 2.18 |
| Rnf19b | protein coding | 44.25 ± 4.21 | 228.31 ± 5.61 | 42.72 ± 0.25 | 229.19 ± 2.76 |
| Htra4 | protein coding | 2.69 ± 0.31 | 27.99 ± 1.18 | 5.66 ± 0.25 | 18.38 ± 0.50 |
| Gca | protein coding | 7.79 ± 1.00 | 57.02 ± 1.21 | 6.14 ± 0.53 | 56.99 ± 2.96 |
| Rgs1 | protein coding | 122.05 ± 16.57 | 805.45 ± 42.41 | 94.50 ± 1.32 | 663.08 ± 14.68 |
| Cflar | protein coding | 37.36 ± 1.75 | 167.15 ± 14.18 | 27.01 ± 0.05 | 141.46 ± 3.37 |
| Ifi214 | protein coding | 6.35 ± 0.48 | 42.29 ± 2.79 | 1.39 ± 0.74 | 34.05 ± 0.78 |
| Sdc4 | protein coding | 4.43 ± 0.15 | 26.44 ± 0.56 | 4.57 ± 0.49 | 24.40 ± 1.61 |
| Lrrc8c | protein coding | 6.58 ± 0.42 | 38.90 ± 4.09 | 7.60 ± 0.38 | 38.99 ± 3.66 |
| Cst7 | protein coding | 4.67 ± 1.22 | 80.82 ± 5.79 | 3.55 ± 0.05 | 63.27 ± 0.18 |
| Abcb1a | protein coding | 0.56 ± 0.13 | 9.92 ± 0.43 | 0.41 ± 0.08 | 5.08 ± 0.04 |
| Nfkbie | protein coding | 16.31 ± 0.89 | 74.61 ± 4.40 | 13.59 ± 0.33 | 60.05 ± 0.82 |
| Gm6545 | processed | 15.20 ± 3.20 | 140.73 ± 8.70 | 3.65 ± 2.15 | 112.77 ± 0.98 |
| | pseudogene | | | | |
| Isg20 | protein coding | 15.59 ± 4.77 | 271.54 ± 19.48 | 5.44 ± 3.01 | 283.47 ± 0.90 |
| Vcan | protein coding | 7.12 ± 1.52 | 64.24 ± 1.61 | 8.29 ± 1.19 | 46.25 ± 0.04 |
| Fabp3 | protein coding | 10.36 ± 1.48 | 81.37 ± 3.29 | 5.79 ± 1.45 | 76.34 ± 1.25 |
| Ptgs2 | protein coding | 0.28 ± 0.09 | 11.71 ± 2.24 | 0.37 ± 0.06 | 11.34 ± 1.29 |
| Ms4a4c | protein coding | 44.59 ± 13.78 | 790.62 ± 102.10 | 15.12 ± 8.78 | 755.03 ± 29.98 |
| Irf7 | protein coding | 138.76 ± 26.57 | 1006.15 ± 30.15 | 47.64 ± 27.05 | 1096.19 ± 17.01 |
| Isg15 | protein coding | 344.72 ± 60.92 | 2316.15 ± 89.40 | 85.16 ± 60.66 | 2370.90 ± 66.79 |
| Basp1 | protein coding | 72.55 ± 1.44 | 279.61 ± 25.06 | 98.57 ± 2.31 | 244.82 ± 2.03 |
| Oasl1 | protein coding | 40.76 ± 11.36 | 542.35 ± 44.42 | 7.89 ± 4.25 | 596.58 ± 0.78 |
| Ccl12 | protein coding | 7.73 ± 1.60 | 109.62 ± 9.65 | 1.30 ± 0.81 | 117.40 ± 0.38 |
| Ptx3 | protein coding | 38.51 ± 2.78 | 189.51 ± 20.34 | 20.12 ± 1.33 | 68.71 ± 2.64 |
| Nfkbia | protein coding | 43.76 ± 3.85 | 171.17 ± 9.45 | 54.49 ± 3.02 | 174.12 ± 11.12 |
| Ifnb1 | protein coding | 0.76 ± 0.09 | 109.56 ± 25.18 | 0.03 ± 0.04 | 120.14 ± 3.12 |
| Mtus1 | protein coding | 5.66 ± 0.65 | 30.80 ± 2.56 | 3.45 ± 0.10 | 24.08 ± 1.68 |
| Gm12185 | protein coding | 1.25 ± 0.16 | 9.98 ± 0.70 | 0.60 ± 0.18 | 8.91 ± 0.95 |
| Prpf38a | protein coding | 18.39 ± 1.62 | 73.02 ± 2.07 | 14.13 ± 1.23 | 75.51 ± 1.48 |
| H2-Q6 | protein coding | 8.25 ± 1.56 | 93.67 ± 17.33 | 13.72 ± 0.76 | 138.17 ± 9.18 |
| Marcksl1 | protein coding | 21.45 ± 4.12 | 201.18 ± 31.92 | 30.89 ± 3.95 | 241.84 ± 8.81 |
| Flt4 | protein coding | 0.40 ± 0.17 | 8.09 ± 0.51 | 0.20 ± 0.11 | 6.67 ± 0.12 |
| Xkr8 | protein coding | 2.71 ± 0.23 | 21.70 ± 3.06 | 1.59 ± 0.54 | 16.95 ± 0.40 |
| C3 | protein coding | 145.19 ± 15.18 | 570.74 ± 38.06 | 214.71 ± 9.68 | 543.89 ± 23.69 |
| Kdr | protein coding | 4.80 ± 1.20 | 42.23 ± 2.77 | 1.69 ± 0.54 | 34.93 ± 1.65 |
| Enpp4 | protein coding | 4.01 ± 0.76 | 24.99 ± 0.39 | 1.71 ± 0.49 | 17.95 ± 0.68 |
| Ccnd2 | protein coding | 19.44 ± 2.42 | 90.49 ± 7.72 | 8.24 ± 1.20 | 75.20 ± 0.41 |
| Il27 | protein coding | 1.46 ± 0.78 | 40.20 ± 2.01 | 0.47 ± 0.11 | 33.64 ± 0.47 |
| Dcbld2 | protein coding | 5.94 ± 0.86 | 28.47 ± 0.94 | 4.79 ± 0.13 | 19.68 ± 0.29 |
| Il18bp | protein coding | 20.15 ± 3.79 | 140.33 ± 15.22 | 19.94 ± 3.49 | 166.80 ± 1.93 |
| Sco1 | protein coding | 5.49 ± 0.53 | 26.69 ± 2.11 | 4.47 ± 0.17 | 29.06 ± 0.25 |
| Scimp | protein coding | 21.62 ± 3.34 | 115.34 ± 7.07 | 8.61 ± 2.67 | 114.64 ± 1.06 |
| Ccrl2 | protein coding | 15.43 ± 4.76 | 179.56 ± 8.85 | 10.32 ± 1.31 | 140.02 ± 4.85 |
| Tmem171 | protein coding | 4.42 ± 0.55 | 41.19 ± 4.94 | 2.58 ± 0.53 | 36.55 ± 1.27 |
| Ctla2b | protein coding | 5.58 ± 1.08 | 48.46 ± 3.50 | 4.07 ± 0.18 | 39.28 ± 1.05 |
| Ccl4 | protein coding | 94.68 ± 6.44 | 381.32 ± 42.24 | 30.65 ± 6.05 | 268.21 ± 9.16 |
| Gm13571 | lncRNA | 2.71 ± 0.59 | 22.64 ± 2.35 | 1.85 ± 0.06 | 13.78 ± 0.75 |
| Adap2 | protein coding | 19.28 ± 1.35 | 74.27 ± 5.95 | 18.72 ± 1.03 | 62.58 ± 1.20 |
| Il1a | protein coding | 9.07 ± 1.37 | 41.92 ± 2.77 | 7.20 ± 0.86 | 13.42 ± 0.10 |
| H2-Q4 | protein coding | 43.51 ± 6.77 | 218.08 ± 16.92 | 43.10 ± 4.01 | 252.87 ± 12.18 |
| Phf11a | protein coding | 24.08 ± 5.69 | 176.00 ± 18.91 | 8.14 ± 4.66 | 185.86 ± 4.46 |
| Phf11d | protein coding | 78.15 ± 11.31 | 316.18 ± 19.98 | 24.85 ± 14.11 | 316.11 ± 2.26 |
| Gbp2 | protein coding | 240.55 ± 53.46 | 1453.24 ± 85.20 | 225.32 ± 37.07 | 1504.30 ± 39.17 |
| Csrnp1 | protein coding | 4.80 ± 0.86 | 25.84 ± 1.03 | 5.61 ± 0.13 | 30.71 ± 3.98 |
| Hcar2 | protein coding | 5.94 ± 0.79 | 29.28 ± 2.04 | 3.86 ± 0.47 | 21.55 ± 0.80 |
| Apol9a | protein coding | 0.92 ± 0.48 | 22.98 ± 3.23 | 0.11 ± 0.04 | 21.59 ± 2.28 |
| Plekha4 | protein coding | 0.71 ± 0.16 | 12.52 ± 1.73 | 0.37 ± 0.01 | 9.67 ± 0.78 |
| Il15ra | protein coding | 10.18 ± 3.10 | 85.79 ± 1.08 | 5.09 ± 0.70 | 68.68 ± 0.45 |
| H2-T22 | protein coding | 115.73 ± 15.79 | 453.67 ± 32.22 | 95.49 ± 13.29 | 546.08 ± 14.86 |
| Usp18 | protein coding | 138.10 ± 28.11 | 685.93 ± 15.83 | 44.89 ± 12.66 | 695.44 ± 0.78 |
| Tnfsf4 | protein coding | 15.98 ± 2.07 | 65.78 ± 6.80 | 14.86 ± 1.40 | 75.71 ± 2.26 |
| Gypc | protein coding | 4.45 ± 0.80 | 24.91 ± 1.43 | 3.46 ± 0.22 | 22.20 ± 0.86 |
| Casp4 | protein coding | 28.63 ± 4.31 | 140.49 ± 17.17 | 14.07 ± 3.08 | 144.28 ± 1.68 |
| Akap12 | protein coding | 0.60 ± 0.08 | 4.08 ± 0.14 | 0.41 ± 0.06 | 3.25 ± 0.12 |
| Heatr9 | protein coding | 0.57 ± 0.32 | 17.41 ± 3.25 | 0.75 ± 0.20 | 24.89 ± 0.82 |
| Slamf8 | protein coding | 46.68 ± 5.35 | 249.64 ± 46.45 | 30.95 ± 5.12 | 186.87 ± 1.93 |
| Hmgn3 | protein coding | 5.26 ± 0.31 | 34.13 ± 2.26 | 5.55 ± 0.18 | 45.66 ± 1.14 |
| Faah | protein coding | 0.75 ± 0.34 | 11.60 ± 1.49 | 0.61 ± 0.11 | 10.08 ± 0.26 |
| Stx11 | protein coding | 2.71 ± 0.84 | 25.02 ± 1.14 | 4.82 ± 0.30 | 24.63 ± 0.08 |
| Tlr3 | protein coding | 17.02 ± 2.94 | 69.72 ± 3.72 | 7.14 ± 2.04 | 53.36 ± 2.89 |
| Nlgn2 | protein coding | 1.09 ± 0.09 | 6.97 ± 0.89 | 0.82 ± 0.01 | 6.56 ± 0.51 |
| Olfr56 | protein coding | 5.54 ± 1.29 | 33.28 ± 1.78 | 1.02 ± 0.63 | 28.18 ± 1.18 |
| Batf2 | protein coding | 9.73 ± 1.86 | 52.67 ± 3.92 | 4.93 ± 1.94 | 50.03 ± 4.98 |
| Tapbpl | protein coding | 12.88 ± 1.61 | 48.93 ± 3.88 | 11.47 ± 2.39 | 59.63 ± 0.94 |
| BC023105 | processed | 0.49 ± 0.27 | 21.62 ± 3.82 | 0.16 ± 0.13 | 27.98 ± 0.65 |
| | pseudogene | | | | |
| Fcgr1 | protein coding | 69.19 ± 13.28 | 298.46 ± 12.74 | 29.68 ± 16.70 | 277.42 ± 3.79 |
| Gbp3 | protein coding | 102.78 ± 20.27 | 430.67 ± 17.26 | 62.01 ± 16.89 | 410.86 ± 6.65 |
| Ifi208 | protein coding | 17.48 ± 4.40 | 96.54 ± 1.93 | 2.87 ± 1.81 | 87.55 ± 0.28 |
| Socs1 | protein coding | 8.72 ± 1.04 | 52.78 ± 11.46 | 2.38 ± 0.24 | 55.73 ± 0.40 |
| Sp140 | protein coding | 64.30 ± 12.70 | 282.16 ± 23.56 | 48.25 ± 8.18 | 310.92 ± 2.08 |
| Phf11b | protein coding | 85.24 ± 21.32 | 466.45 ± 36.89 | 34.36 ± 16.96 | 494.58 ± 1.11 |
| Spta1 | protein coding | 0.04 ± 0.02 | 2.51 ± 0.17 | 0.02 ± 0.01 | 1.39 ± 0.03 |
| Cd86 | protein coding | 20.23 ± 5.25 | 126.40 ± 16.15 | 16.91 ± 3.06 | 140.67 ± 0.06 |
| Clic5 | protein coding | 0.04 ± 0.01 | 5.92 ± 1.91 | 0.02 ± 0.01 | 7.06 ± 1.10 |
| Il12rb1 | protein coding | 0.33 ± 0.03 | 6.47 ± 1.09 | 0.35 ± 0.03 | 6.66 ± 0.03 |
| Serpinb2 | protein coding | 2.73 ± 0.18 | 19.69 ± 4.04 | 3.35 ± 0.99 | 14.65 ± 0.19 |
| Cfb | protein coding | 84.51 ± 21.43 | 641.87 ± 140.10 | 131.37 ± 8.09 | 843.88 ± 33.40 |
| Il23r | protein coding | 0.65 ± 0.16 | 9.48 ± 2.07 | 0.58 ± 0.07 | 9.83 ± 0.14 |
| Gnb4 | protein coding | 11.39 ± 2.03 | 52.10 ± 5.41 | 8.14 ± 0.69 | 54.92 ± 5.47 |
| H2-Q7 | protein coding | 7.82 ± 2.28 | 70.01 ± 15.12 | 19.97 ± 0.47 | 112.89 ± 6.62 |
| Ifit2 | protein coding | 358.84 ± 104.49 | 2096.85 ± 93.25 | 61.50 ± 41.42 | 1938.71 ± 34.16 |
| Cxcl11 | polymorphic | 0.07 ± 0.07 | 22.47 ± 4.36 | 0.04 ± 0.01 | 19.95 ± 1.71 |
| | pseudogene | | | | |
| Gbp6 | protein coding | 45.98 ± 10.94 | 235.61 ± 26.66 | 16.66 ± 7.64 | 233.23 ± 2.69 |
| Hbegf | protein coding | 5.83 ± 0.67 | 32.26 ± 6.14 | 3.52 ± 0.30 | 18.35 ± 1.56 |
| Pou3f1 | protein coding | 0.83 ± 0.14 | 7.89 ± 1.72 | 0.25 ± 0.04 | 9.06 ± 0.46 |
| Upp1 | protein coding | 5.60 ± 1.29 | 38.14 ± 5.40 | 2.16 ± 0.34 | 18.25 ± 1.82 |
| Trim30c | protein coding | 13.74 ± 4.18 | 85.17 ± 1.86 | 2.68 ± 1.14 | 62.00 ± 2.14 |
| Rilpl1 | protein coding | 5.98 ± 1.43 | 31.12 ± 2.23 | 2.47 ± 0.28 | 25.21 ± 0.64 |
| A530040E14Rik | lncRNA | 4.24 ± 1.32 | 32.02 ± 2.64 | 2.59 ± 1.56 | 45.89 ± 1.64 |
| Gpr55 | protein coding | 1.63 ± 0.23 | 8.65 ± 1.16 | 2.84 ± 0.21 | 12.02 ± 0.21 |
| Il1rn | protein coding | 467.00 ± 93.15 | 1888.93 ± 180.49 | 307.09 ± 5.01 | 1398.94 ± 40.57 |
| Ccl7 | protein coding | 17.85 ± 2.29 | 73.55 ± 7.32 | 7.64 ± 0.68 | 71.17 ± 7.32 |
| Rgl1 | protein coding | 16.82 ± 2.54 | 64.31 ± 8.43 | 8.71 ± 1.26 | 38.54 ± 0.23 |
| Il15 | protein coding | 31.98 ± 7.00 | 146.03 ± 15.04 | 21.22 ± 3.66 | 146.44 ± 3.73 |
| Tagap | protein coding | 3.41 ± 0.47 | 16.73 ± 2.66 | 1.31 ± 0.06 | 11.12 ± 0.52 |
| 4933412E12Rik | lncRNA | 2.49 ± 0.15 | 13.24 ± 0.58 | 1.21 ± 0.02 | 12.93 ± 0.84 |
| Mx2 | polymorphic | 32.62 ± 7.53 | 139.48 ± 10.97 | 4.16 ± 2.11 | 100.25 ± 0.34 |
| | pseudogene | | | | |
| C130026I21Rik | protein coding | 12.79 ± 3.59 | 80.38 ± 9.30 | 15.09 ± 3.56 | 120.46 ± 8.42 |
| Ifih1 | protein coding | 65.30 ± 15.87 | 281.75 ± 18.39 | 25.92 ± 7.18 | 233.12 ± 2.59 |
| Gm14569 | protein coding | 0.13 ± 0.08 | 3.61 ± 0.33 | 0.05 ± 0.03 | 3.01 ± 0.31 |
| Slc28a2 | protein coding | 3.36 ± 0.87 | 18.77 ± 1.66 | 1.89 ± 0.40 | 13.19 ± 0.86 |
| Il6 | protein coding | 0.39 ± 0.23 | 21.94 ± 7.64 | 0.15 ± 0.09 | 34.35 ± 0.45 |
| Igtp | protein coding | 37.98 ± 9.09 | 157.03 ± 8.31 | 11.89 ± 4.44 | 152.72 ± 0.89 |
| Serpina3g | protein coding | 14.13 ± 5.56 | 487.37 ± 37.08 | 6.30 ± 1.61 | 386.79 ± 19.58 |
| Rab20 | protein coding | 5.50 ± 0.65 | 22.37 ± 1.62 | 4.69 ± 0.57 | 17.13 ± 0.33 |
| Ddx4 | protein coding | 0.85 ± 0.23 | 6.20 ± 0.33 | 0.26 ± 0.05 | 5.31 ± 0.28 |
| A630012P03Rik | lncRNA | 0.44 ± 0.08 | 6.54 ± 0.89 | 0.16 ± 0.16 | 6.31 ± 0.16 |
| Slc39a2 | protein coding | 3.44 ± 0.45 | 12.87 ± 0.87 | 1.63 ± 0.05 | 10.00 ± 0.59 |
| Abtb2 | protein coding | 2.11 ± 0.63 | 11.63 ± 0.59 | 0.95 ± 0.12 | 11.70 ± 0.11 |
| Nod1 | protein coding | 8.32 ± 1.77 | 32.09 ± 2.27 | 4.18 ± 0.68 | 28.55 ± 0.13 |
| Angpt1 | protein coding | 0.08 ± 0.04 | 2.77 ± 0.21 | 0.06 ± 0.05 | 1.53 ± 0.14 |
| Wnk2 | protein coding | 0.94 ± 0.20 | 5.22 ± 0.63 | 0.86 ± 0.10 | 5.22 ± 0.51 |
| Gbp11 | polymorphic | 1.00 ± 0.33 | 8.39 ± 0.95 | 0.28 ± 0.13 | 7.75 ± 0.43 |
| | pseudogene | | | | |
| Rasgrp1 | protein coding | 4.30 ± 0.68 | 19.51 ± 4.40 | 4.33 ± 0.71 | 18.18 ± 0.67 |
| Hdc | protein coding | 0.92 ± 0.21 | 8.36 ± 1.07 | 0.30 ± 0.17 | 5.20 ± 0.23 |
| Mtmr7 | protein coding | 2.06 ± 0.08 | 7.83 ± 0.24 | 2.17 ± 0.33 | 5.60 ± 0.16 |
| Ifit1 | protein coding | 163.70 ± 49.38 | 742.87 ± 22.18 | 23.29 ± 11.05 | 742.74 ± 3.40 |
| Slfn4 | protein coding | 8.75 ± 2.74 | 150.22 ± 24.71 | 1.88 ± 1.51 | 119.07 ± 0.26 |
| Cd40 | protein coding | 9.82 ± 3.89 | 254.00 ± 16.48 | 5.70 ± 1.97 | 238.78 ± 10.93 |
| Cd38 | protein coding | 2.72 ± 0.46 | 13.62 ± 2.90 | 2.98 ± 0.39 | 8.83 ± 0.16 |
| Naip1 | protein coding | 0.88 ± 0.04 | 3.92 ± 0.53 | 1.43 ± 0.11 | 4.83 ± 0.27 |
| Fgfbp3 | protein coding | 1.71 ± 0.31 | 9.40 ± 0.65 | 1.07 ± 0.19 | 8.79 ± 0.49 |
| Gbp10 | protein coding | 0.57 ± 0.21 | 7.48 ± 0.55 | 0.17 ± 0.19 | 7.02 ± 0.42 |
| Ifi44 | protein coding | 57.73 ± 18.55 | 279.20 ± 26.48 | 16.95 ± 11.58 | 307.24 ± 3.92 |
| Mir155hg | lncRNA | 2.52 ± 0.26 | 20.46 ± 5.73 | 1.75 ± 0.35 | 14.80 ± 0.00 |
| Cxcl9 | protein coding | 1.13 ± 0.63 | 145.16 ± 36.78 | 0.84 ± 0.61 | 174.13 ± 1.19 |
| Pla1a | protein coding | 0.27 ± 0.14 | 5.55 ± 0.20 | 0.27 ± 0.03 | 3.87 ± 0.08 |
| Ifit3 | protein coding | 371.39 ± 107.91 | 1462.22 ± 19.21 | 49.92 ± 31.83 | 1258.85 ± 21.79 |
| Pakap | protein coding | 2.00 ± 0.43 | 9.63 ± 1.86 | 3.22 ± 0.59 | 13.30 ± 0.36 |
| Bambi-ps1 | processed | 2.08 ± 0.67 | 24.00 ± 1.31 | 1.12 ± 0.44 | 20.91 ± 0.51 |
| | pseudogene | | | | |
| Asb11 | protein coding | 2.98 ± 0.16 | 13.10 ± 1.45 | 2.80 ± 0.93 | 14.94 ± 0.38 |
| Socs3 | protein coding | 2.78 ± 0.27 | 10.71 ± 1.44 | 2.46 ± 0.08 | 9.17 ± 0.54 |
| Tnfsf18 | protein coding | 0.59 ± 0.14 | 5.31 ± 0.36 | 0.60 ± 0.04 | 2.87 ± 0.13 |
| Armcx6 | protein coding | 0.56 ± 0.17 | 5.38 ± 0.20 | 0.31 ± 0.03 | 3.37 ± 0.34 |
| Ch25h | protein coding | 2.73 ± 1.09 | 81.27 ± 12.40 | 1.40 ± 0.40 | 79.47 ± 3.10 |
| Lhx2 | protein coding | 0.72 ± 0.35 | 7.20 ± 0.75 | 0.16 ± 0.14 | 6.50 ± 0.06 |
| Ccdc39 | protein coding | 0.97 ± 0.15 | 5.21 ± 0.29 | 0.28 ± 0.24 | 2.43 ± 0.03 |
| Trp53i11 | protein coding | 1.55 ± 0.68 | 13.37 ± 1.83 | 0.94 ± 0.47 | 13.83 ± 0.10 |
| Morrbid | lncRNA | 7.43 ± 1.81 | 31.35 ± 0.79 | 3.51 ± 0.54 | 32.14 ± 3.38 |
| Ccdc173 | protein coding | 1.25 ± 0.25 | 7.18 ± 0.37 | 0.66 ± 0.01 | 7.78 ± 0.15 |
| Traf1 | protein coding | 25.26 ± 5.42 | 102.73 ± 20.79 | 39.72 ± 1.63 | 139.03 ± 4.86 |
| Ifi205 | protein coding | 28.82 ± 11.14 | 556.02 ± 60.28 | 4.14 ± 2.45 | 499.87 ± 10.49 |
| Tnn | protein coding | 0.03 ± 0.02 | 1.80 ± 0.16 | 0.19 ± 0.04 | 0.92 ± 0.02 |
| AC147806.2 | protein coding | 1.20 ± 0.50 | 8.85 ± 0.45 | 0.92 ± 0.40 | 14.17 ± 0.32 |
| Gm34643 | lncRNA | 1.39 ± 0.43 | 8.56 ± 0.96 | 0.82 ± 0.05 | 7.34 ± 0.66 |
| Edn1 | protein coding | 4.41 ± 0.56 | 18.23 ± 3.94 | 2.15 ± 0.16 | 10.84 ± 0.80 |
| Ifna2 | protein coding | 0.12 ± 0.11 | 24.18 ± 4.29 | 0.00 ± 0.00 | 22.99 ± 0.05 |
| A530032D15Rik | protein coding | 1.97 ± 0.59 | 13.14 ± 1.12 | 2.31 ± 0.43 | 17.61 ± 0.06 |
| Cnn3 | protein coding | 1.91 ± 0.15 | 8.20 ± 1.07 | 3.81 ± 0.58 | 8.87 ± 0.64 |
| Bmp10 | protein coding | 0.08 ± 0.06 | 2.05 ± 0.19 | 0.02 ± 0.01 | 1.72 ± 0.20 |
| Dmtn | protein coding | 0.32 ± 0.06 | 3.30 ± 0.32 | 0.57 ± 0.17 | 3.85 ± 0.23 |
| Rnf225 | protein coding | 0.05 ± 0.02 | 3.09 ± 0.43 | 0.04 ± 0.01 | 2.59 ± 0.19 |
| Alpk2 | protein coding | 1.11 ± 0.15 | 4.74 ± 0.34 | 1.16 ± 0.14 | 3.83 ± 0.37 |
| Uaca | protein coding | 2.36 ± 0.29 | 10.35 ± 2.59 | 2.14± 0.20 | 5.25 ± 0.00 |
| Neb | protein coding | 0.07 ± 0.04 | 1.33 ± 0.24 | 0.08 ± 0.01 | 0.92 ± 0.06 |
| Gem | protein coding | 0.86 ± 0.37 | 8.87 ± 2.07 | 0.77 ± 0.08 | 8.05 ± 0.29 |
| Tlr9 | protein coding | 10.39 ± 3.40 | 42.21 ± 1.81 | 6.58 ± 1.72 | 46.37 ± 0.70 |
| Gm7609 | protein coding | 1.13 ± 0.44 | 11.36 ± 2.46 | 1.15 ± 0.65 | 18.49 ± 0.07 |
| Rgcc | protein coding | 8.19 ± 0.93 | 31.75 ± 5.30 | 8.25 ± 0.74 | 35.74 ± 1.79 |
| Gm47662 | lncRNA | 0.13 ± 0.06 | 2.59 ± 0.60 | 0.04 ± 0.04 | 1.85 ± 0.08 |
| 9530082P21Rik | lncRNA | 2.16 ± 0.53 | 8.48 ± 0.05 | 1.49 ± 0.00 | 9.25 ± 0.27 |
| Fpr2 | protein coding | 24.05 ± 5.83 | 101.33 ± 21.13 | 23.78 ± 0.78 | 91.66 ± 1.02 |
| Arid5a | protein coding | 9.80 ± 3.45 | 44.29 ± 4.36 | 3.50 ± 0.90 | 40.81 ± 4.36 |
| Pde11a | protein coding | 0.59 ± 0.22 | 5.25 ± 1.19 | 0.69 ± 0.21 | 4.43 ± 0.34 |
| Gm43302 | protein coding | 0.13 ± 0.10 | 2.17 ± 0.07 | 0.05 ± 0.06 | 1.98 ± 0.83 |
| Cmpk2 | protein coding | 144.22 ± 49.92 | 1489.01 ± 44.79 | 18.96 ± 6.55 | 1148.63 ± 31.76 |
| Adora2a | protein coding | 2.15 ± 0.73 | 10.47 ± 0.30 | 4.12 ± 1.25 | 13.60 ± 0.60 |
| Lipg | protein coding | 0.29 ± 0.08 | 2.28 ± 0.17 | 0.07 ± 0.03 | 1.80 ± 0.01 |
| Sdcbp2 | protein coding | 0.96 ± 0.08 | 7.23 ± 1.20 | 1.93 ± 0.07 | 8.82 ± 0.28 |
| Il12b | protein coding | 0.36 ± 0.21 | 32.60 ± 12.37 | 1.27 ± 0.13 | 63.03 ± 2.44 |
| Gbp5 | protein coding | 44.24 ± 14.65 | 391.65 ± 23.30 | 12.59 ± 4.70 | 348.17 ± 3.75 |
| Hrh2 | protein coding | 1.07 ± 0.22 | 4.61 ± 0.58 | 0.50 ± 0.14 | 4.90 ± 0.45 |
| Rsad2 | protein coding | 277.94 ± 92.79 | 2354.19 ± 23.05 | 36.74 ± 16.34 | 2012.10 ± 47.33 |
| Trim30b | protein coding | 3.59 ± 1.22 | 14.50 ± 1.40 | 0.75 ± 0.28 | 13.12 ± 0.99 |
| AW011738 | lncRNA | 3.38 ± 1.19 | 16.65 ± 3.24 | 1.11 ± 0.51 | 16.54 ± 0.73 |
| Sell | protein coding | 1.16 ± 0.25 | 5.04 ± 0.37 | 0.57 ± 0.20 | 4.75 ± 1.28 |
| Cd69 | protein coding | 24.97 ± 10.02 | 321.75 ± 16.10 | 2.32 ± 0.66 | 250.95 ± 5.01 |
| Gm21860 | lncRNA | 5.13 ± 1.97 | 34.31 ± 9.52 | 2.85 ± 0.71 | 29.00 ± 1.95 |
| Mmp25 | protein coding | 5.40 ± 1.23 | 22.38 ± 6.30 | 11.45 ± 0.23 | 37.89 ± 0.76 |
| Adhfe1 | protein coding | 1.13 ± 0.03 | 5.04 ± 0.61 | 1.04 ± 0.13 | 6.04 ± 0.06 |
| Gm8221 | unprocessed | 0.29 ± 0.05 | 12.28 ± 6.59 | 0.24 ± 0.11 | 16.24 ± 0.45 |
| | pseudogene | | | | |
| Lta | protein coding | 0.19 ± 0.09 | 4.65 ± 0.20 | 0.09 ± 0.01 | 2.82 ± 0.29 |
| Aplnr | protein coding | 0.26 ± 0.10 | 2.60 ± 0.70 | 0.21 ± 0.03 | 1.66 ± 0.05 |
| Gm16685 | lncRNA | 0.55 ± 0.09 | 4.60 ± 0.64 | 1.32 ± 0.04 | 7.31 ± 0.20 |
| 2310043P16Rik | TEC | 1.36 ± 0.45 | 5.41 ± 0.21 | 0.70 ± 0.34 | 4.41 ± 0.42 |
| Cd226 | protein coding | 6.23 ± 2.46 | 28.57 ± 1.90 | 4.57 ± 1.27 | 21.64 ± 0.08 |
| Gm47242 | lncRNA | 2.03 ± 0.30 | 23.90 ± 5.61 | 0.58 ± 0.47 | 18.23 ± 1.37 |
| Glrp1 | protein coding | 1.25 ± 0.11 | 5.36 ± 0.67 | 0.71 ± 0.14 | 4.74 ± 0.07 |
| H2-Q5 | polymorphic | 3.79 ± 1.20 | 26.25 ± 9.56 | 6.27 ± 2.22 | 23.20 ± 3.15 |
| | pseudogene | | | | |
| Lpar1 | protein coding | 0.27 ± 0.07 | 2.17 ± 0.32 | 0.44 ± 0.17 | 1.94 ± 0.16 |
| Il1b | protein coding | 2.15 ± 0.72 | 11.17 ± 1.51 | 1.84 ± 0.21 | 9.44 ± 0.35 |
| Atp13a4 | protein coding | 1.88 ± 0.77 | 10.96 ± 2.82 | 1.90 ± 0.65 | 7.85 £ 1.70 |
| Gm12551 | unprocessed | 1.47 ± 0.80 | 14.89 ± 2.10 | 1.05 ± 0.82 | 21.58 ± 1.73 |
| | pseudogene | | | | |
| Asap3 | protein coding | 0.05 ± 0.03 | 1.33 ± 0.23 | 0.14 ± 0.02 | 1.41 ± 0.14 |
| Sectm1a | protein coding | 2.51 ± 0.61 | 9.79 ± 1.24 | 1.09 ± 0.57 | 6.51 ± 0.11 |
| Hspa1a | protein coding | 1.22 ± 0.31 | 7.45 ± 2.81 | 1.59 ± 0.01 | 4.97 ± 0.06 |
| Cxcl10 | protein coding | 71.79 ± 37.00 | 1560.26 ± 79.60 | 5.49 ± 2.02 | 1205.22 ± 35.48 |
| Ubd | protein coding | 0.28 ± 0.07 | 6.80 ± 1.13 | 0.25 ± 0.08 | 5.22 ± 0.27 |
| Ly6i | protein coding | 12.01 ± 3.05 | 55.63 ± 19.75 | 15.88 ± 2.05 | 88.53 ± 5.73 |
| 9130230L23Rik | protein coding | 1.04 ± 0.19 | 4.63 ± 0.64 | 1.62 ± 0.16 | 3.72 ± 0.27 |
| Gpr33 | protein coding | 0.24 ± 0.07 | 3.84 ± 0.26 | 0.03 ± 0.00 | 2.78 ± 0.37 |
| Hspa1b | protein coding | 1.67 ± 0.23 | 9.18 ± 3.93 | 1.75 ± 0.05 | 7.52 ± 0.62 |
| Ifna5 | protein coding | 0.12 ± 0.15 | 42.62 ± 7.77 | 0.00 ± 0.00 | 32.00 ± 0.87 |
| Gpr31c | transcribed | 0.43 ± 0.06 | 1.80 ± 0.17 | 0.50 ± 0.08 | 2.33 ± 0.03 |
| | processed | | | | |
| | pseudogene | | | | |
| Oprd1 | protein coding | 0.16 ± 0.05 | 1.48 ± 0.34 | 0.37 ± 0.08 | 2.23 ± 0.11 |
| Slc17a8 | protein coding | 0.44 ± 0.16 | 2.29 ± 0.21 | 0.38 ± 0.04 | 1.82 ± 0.21 |
| AW112010 | lncRNA | 223.31 ± 74.24 | 1754.30 ± 351.70 | 255.32 ± 48.27 | 2367.08 ± 77.65 |
| Gm41442 | lncRNA | 0.28 ± 0.04 | 1.41 ± 0.16 | 0.12 ± 0.01 | 1.11 ± 0.01 |
| Kalrn | protein coding | 0.29 ± 0.06 | 1.64 ± 0.35 | 0.53 ± 0.13 | 0.73 ± 0.00 |
| Ccl8 | protein coding | 0.42 ± 0.22 | 15.01 ± 2.59 | 0.26 ± 0.06 | 10.40 ± 0.76 |
| Clcn1 | protein coding | 0.45 ± 0.13 | 2.49 ± 0.46 | 0.18 ± 0.04 | 1.78 ± 0.33 |
| Calhm6 | protein coding | 9.97 ± 4.77 | 115.54 ± 9.76 | 4.07 ± 0.93 | 85.38 ± 0.63 |
| Gm10553 | lncRNA | 0.25 ± 0.12 | 3.32 ± 0.52 | 0.36 ± 0.10 | 5.67 ± 0.44 |
| Ifit3b | protein coding | 47.54 ± 17.02 | 303.37 ± 10.23 | 5.11 ± 3.13 | 262.43 ± 8.00 |
| Col27a1 | protein coding | 0.38 ± 0.18 | 2.58 ± 0.61 | 0.51 ± 0.13 | 2.75 ± 0.35 |
| B230303A05Rik | transcribed | 0.20 ± 0.12 | 2.54 ± 0.30 | 0.06 ± 0.01 | 2.53 ± 0.57 |
| | processed | | | | |
| | pseudogene | | | | |
| AC167036.1 | protein coding | 0.57 ± 0.01 | 4.55 ± 0.47 | 0.48 ± 0.26 | 8.11 ± 0.37 |
| Kcnab1 | protein coding | 0.83 ± 0.19 | 3.39 ± 0.51 | 0.65 ± 0.13 | 3.28 ± 0.08 |
| Phf11 | unprocessed | 1.75 ± 0.51 | 11.45 ± 0.66 | 0.60 ± 0.19 | 13.66 ± 0.34 |
| | pseudogene | | | | |
| Apol7c | protein coding | 7.82 ± 2.56 | 115.62 ± 48.11 | 27.11 ± 2.81 | 176.09 ± 3.96 |
| Ifit1bl1 | protein coding | 84.40 ± 45.15 | 1328.06 ± 85.27 | 11.49 ± 6.88 | 1203.74 ± 39.89 |
| Tgtp1 | protein coding | 0.91 ± 0.82 | 84.55 ± 21.07 | 0.24 ± 0.18 | 100.99 ± 2.49 |
| 9130015A21Rik | lncRNA | 1.50 ± 0.32 | 11.21 ± 2.17 | 1.81 ± 0.08 | 9.75 ± 1.47 |
| Gdf11 | protein coding | 0.02 ± 0.01 | 1.23 ± 0.11 | 0.19 ± 0.03 | 1.50 ± 0.11 |
| 4930599N23Rik | lncRNA | 1.12 ± 0.25 | 6.51 ± 0.29 | 0.87 ± 0.09 | 7.05 ± 0.07 |
| Jam2 | protein coding | 0.08 ± 0.04 | 1.44 ± 0.11 | 0.13 ± 0.08 | 1.06 ± 0.04 |
| Iigp1 | protein coding | 19.96 ± 10.78 | 301.92 ± 32.12 | 3.68 ± 2.18 | 286.93 ± 3.89 |
| Adgb | protein coding | 0.26 ± 0.04 | 1.42 ± 0.20 | 0.21 ± 0.04 | 0.80 ± 0.25 |
| A330040F15Rik | lncRNA | 0.30 ± 0.10 | 2.46 ± 0.50 | 0.05 ± 0.01 | 2.00 ± 0.18 |
| Gbp4 | protein coding | 18.14 ± 8.47 | 169.63 ± 11.05 | 4.76 ± 2.02 | 155.33 ± 0.64 |
| Ifna4 | protein coding | 0.14 ± 0.15 | 20.68 ± 7.51 | 0.00 ± 0.00 | 29.06 ± 1.14 |
| Gm38025 | lncRNA | 0.29 ± 0.09 | 2.01 ± 0.37 | 0.30 ± 0.13 | 3.37 ± 0.21 |
| Cldn23 | protein coding | 0.07 ± 0.02 | 1.90 ± 0.15 | 0.06 ± 0.00 | 0.91 ± 0.06 |
| Tnfsf10 | protein coding | 6.60 ± 2.56 | 36.49 ± 1.49 | 1.30 ± 0.28 | 31.70 ± 0.17 |
| Gm43814 | TEC | 1.10 ± 0.45 | 15.13 ± 2.56 | 0.98 ± 0.47 | 10.59 ± 1.66 |
| Gm11992 | protein coding | 0.08 ± 0.03 | 1.33 ± 0.15 | 0.10 ± 0.01 | 0.93 ± 0.06 |
| Gm16181 | protein coding | 1.43 ± 0.66 | 10.05 ± 0.66 | 1.58 ± 0.21 | 6.32 ± 1.12 |
| Cited4 | protein coding | 2.39 ± 0.70 | 11.66 ± 4.66 | 2.04 ± 0.23 | 8.92 ± 0.42 |
| Tgtp2 | protein coding | 29.20 ± 14.67 | 285.50 ± 21.87 | 5.91 ± 3.57 | 293.22 ± 5.70 |
| Misp | protein coding | 0.56 ± 0.20 | 2.72 ± 0.34 | 0.20 ± 0.12 | 3.02 ± 0.14 |
| A730011C13Rik | lncRNA | 0.56 ± 0.09 | 2.92 ± 0.61 | 0.38 ± 0.08 | 2.53 ± 0.23 |
| Gm10552 | lncRNA | 2.23 ± 0.69 | 19.58 ± 4.42 | 0.56 ± 0.61 | 21.27 ± 0.04 |
| Gm50083 | processed | 25.60 ± 2.83 | 144.77 ± 33.56 | 27.16 ± 5.92 | 177.70 ± 14.76 |
| | pseudogene | | | | |
| Timd4 | protein coding | 0.12 ± 0.05 | 1.69 ± 0.53 | 0.21 ± 0.11 | 0.73 ± 0.06 |
| 1110002J07Rik | lncRNA | 0.40 ± 0.21 | 3.63 ± 1.60 | 0.36 ± 0.23 | 3.77 ± 0.28 |
| Spata31d1b | protein coding | 0.12 ± 0.03 | 1.22 ± 0.56 | 0.29 ± 0.10 | 2.55 ± 0.40 |
| Dennd6b | protein coding | 0.39 ± 0.04 | 1.76 ± 0.36 | 0.47 ± 0.02 | 1.27 ± 0.19 |
| Dnmt3c | protein coding | 0.17 ± 0.10 | 1.69 ± 0.24 | 0.07 ± 0.10 | 1.40 ± 0.09 |
| Fap | protein coding | 0.56 ± 0.24 | 2.33 ± 0.03 | 0.31 ± 0.08 | 2.64 ± 0.30 |
| Gm45193 | lncRNA | 0.20 ± 0.06 | 2.26 ± 0.67 | 0.24 ± 0.19 | 1.41 ± 0.10 |
| Chrna5 | protein coding | 0.51 ± 0.07 | 2.19 ± 0.19 | 0.14 ± 0.00 | 1.83 ± 0.25 |
| Plekha7 | protein coding | 0.15 ± 0.08 | 1.07 ± 0.23 | 0.33 ± 0.07 | 0.70 ± 0.08 |
| Fgl2 | protein coding | 73.38 ± 29.11 | 394.86 ± 47.89 | 43.45 ± 12.83 | 403.80 ± 17.62 |
| Hap1 | protein coding | 0.42 ± 0.11 | 1.81 ± 0.33 | 0.34 ± 0.20 | 2.23 ± 0.33 |
| Gbp2b | protein coding | 1.77 ± 1.02 | 24.74 ± 6.64 | 1.74 ± 0.40 | 32.78 ± 0.95 |
| Ifna9 | protein coding | 0.05 ± 0.08 | 18.41 ± 2.03 | 0.00 ± 0.00 | 17.81 ± 3.05 |
| Gm29094 | protein coding | 2.87 ± 0.83 | 11.33 ± 2.22 | 2.23 ± 1.07 | 12.69 ± 1.65 |
| Tspan33 | protein coding | 1.14 ± 0.38 | 4.66 ± 1.43 | 2.57 ± 0.36 | 7.85 ± 0.27 |
| Plat | protein coding | 0.12 ± 0.07 | 1.49 ± 0.66 | 0.70 ± 0.09 | 2.66 ± 0.23 |
| Arhgap28 | protein coding | 0.39 ± 0.08 | 1.83 ± 0.77 | 0.47 ± 0.10 | 2.11 ± 0.07 |
| Coch | protein coding | 0.05 ± 0.03 | 1.00 ± 0.24 | 0.01 ± 0.01 | 0.80 ± 0.16 |
| Ifna6 | protein coding | 0.02 ± 0.04 | 14.93 ± 2.76 | 0.00 ± 0.00 | 15.23 ± 1.84 |
| Gm4841 | protein coding | 0.05 ± 0.07 | 3.34 ± 0.88 | 0.05 ± 0.00 | 4.74 ± 0.18 |
| Gm15726 | lncRNA | 2.55 ± 1.30 | 12.82 ± 1.89 | 1.30 ± 0.40 | 4.08 ± 0.48 |
| Lad1 | protein coding | 0.45 ± 0.15 | 2.06 ± 0.70 | 0.88 ± 0.04 | 2.99 ± 0.33 |
| Gm16094 | lncRNA | 2.79 ± 1.35 | 15.35 ± 3.13 | 3.05 ± 0.64 | 21.50 ± 2.86 |
| Ccr7 | protein coding | 29.95 ± 8.58 | 138.58 ± 46.32 | 84.96 ± 1.12 | 277.99 ± 1.56 |
| Gm12764 | lncRNA | 0.73 ± 0.20 | 5.37 ± 0.15 | 0.27 ± 0.27 | 4.18 ± 0.22 |
| Ifna11 | protein coding | 0.00 ± 0.00 | 7.39 ± 2.44 | 0.00 ± 0.00 | 5.55 ± 0.49 |
| Gm8773 | protein coding | 0.22 ± 0.08 | 2.07 ± 0.51 | 0.19 ± 0.03 | 3.28 ± 0.42 |
| 4933432I03Rik | lncRNA | 0.29 ± 0.01 | 2.56 ± 0.67 | 0.32 ± 0.08 | 2.27 ± 0.04 |
| Gm19076 | processed | 0.64 ± 0.08 | 4.40 ± 0.59 | 0.59 ± 0.34 | 3.92 ± 0.40 |
| | pseudogene | | | | |
| Gm12474 | lncRNA | 0.64 ± 0.22 | 2.94 ± 0.66 | 0.30 ± 0.04 | 2.78 ± 0.01 |
| Gm49673 | protein coding | 0.09 ± 0.09 | 1.01 ± 0.22 | 0.27 ± 0.17 | 2.12 ± 0.43 |
| Fscn1 | protein coding | 17.30 ± 4.25 | 77.32 ± 27.04 | 38.61 ± 2.09 | 148.83 ± 5.63 |
| Efna2 | protein coding | 0.20 ± 0.07 | 1.57 ± 0.68 | 0.76 ± 0.30 | 2.49 ± 0.23 |
| Gm12840 | lncRNA | 0.28 ± 0.10 | 4.58 ± 1.87 | 0.65 ± 0.05 | 4.78 ± 0.35 |
| Cyp2c69 | protein coding | 0.48 ± 0.19 | 2.13 ± 0.40 | 0.27 ± 0.31 | 2.81 ± 0.16 |
| Cadps | protein coding | 0.17 ± 0.09 | 1.38 ± 0.44 | 0.60 ± 0.40 | 1.31 ± 0.31 |
| Ifna1 | protein coding | 0.02 ± 0.04 | 8.45 ± 2.86 | 0.00 ± 0.00 | 10.05 ± 0.98 |
| Apod | protein coding | 0.31 ± 0.33 | 2.87 ± 0.60 | 0.27 ± 0.03 | 3.79 ± 1.22 |
| Dll1 | protein coding | 0.49 ± 0.28 | 2.07 ± 0.25 | 0.07 ± 0.01 | 1.71 ± 0.06 |
| Fam3b | protein coding | 0.25 ± 0.10 | 2.63 ± 0.80 | 0.36 ± 0.04 | 3.22 ± 0.48 |
| Gstt1 | protein coding | 0.84 ± 0.32 | 3.38 ± 0.37 | 1.03 ± 0.26 | 4.53 ± 0.65 |
| Il2ra | protein coding | 1.29 ± 0.32 | 6.72 ± 2.81 | 0.86 ± 0.01 | 11.55 ± 0.27 |
| Gm14199 | lncRNA | 0.23 ± 0.26 | 5.32 ± 0.72 | 0.00 ± 0.00 | 6.53 ± 0.01 |
| Ptgs2os2 | lncRNA | 0.34 ± 0.03 | 1.53 ± 0.36 | 0.15 ± 0.01 | 1.89 ± 0.57 |
| Gm26902 | lncRNA | 0.17 ± 0.11 | 1.19 ± 0.27 | 0.10 ± 0.00 | 1.57 ± 0.01 |
| Ifna14 | protein coding | 0.02 ± 0.04 | 5.75 ± 1.03 | 0.00 ± 0.00 | 5.06 ± 0.51 |
| Gm15998 | lncRNA | 0.01 ± 0.02 | 1.34 ± 0.38 | 0.01 ± 0.01 | 0.56 ± 0.09 |
| G530011O06Rik | lncRNA | 5.13 ± 3.16 | 44.42 ± 17.80 | 0.81 ± 0.27 | 24.97 ± 1.78 |
| Cp | protein coding | 0.37 ± 0.10 | 2.02 ± 1.43 | 0.54 ± 0.21 | 1.72 ± 0.19 |
| Klri1 | protein coding | 0.13 ± 0.09 | 2.44 ± 0.84 | 0.25 ± 0.21 | 2.12 ± 0.60 |
| Gm21748 | protein coding | 0.02 ± 0.03 | 11.98 ± 4.22 | 0.29 ± 0.40 | 5.37 ± 0.65 |
| Plag11 | protein coding | 0.24 ± 0.25 | 5.43 ± 0.66 | 0.15 ± 0.04 | 2.18 ± 0.10 |
| Gm46189 | lncRNA | 0.68 ± 0.14 | 3.95 ± 0.24 | 0.19 ± 0.04 | 2.82 ± 0.08 |
| Ifnab | protein coding | 0.02 ± 0.03 | 2.29 ± 0.65 | 0.00 ± 0.00 | 2.06 ± 0.40 |
| Gm12216 | protein coding | 0.97 ± 0.20 | 4.02 ± 0.68 | 0.43 ± 0.13 | 4.10 ± 1.23 |
| A930015D03Rik | lncRNA | 0.26 ± 0.09 | 1.74 ± 0.23 | 0.24 ± 0.06 | 1.82 ± 0.16 |
| Gm49662 | lncRNA | 0.15 ± 0.19 | 4.37 ± 1.32 | 0.10 ± 0.13 | 3.12 ± 0.40 |
| Ifna15 | protein coding | 0.05 ± 0.08 | 4.69 ± 1.17 | 0.04 ± 0.06 | 5.60 ± 0.11 |
| Gm19684 | protein coding | 0.36 ± 0.09 | 1.50 ± 0.31 | 0.25 ± 0.07 | 1.50 ± 0.13 |
| Gm28809 | lncRNA | 0.32 ± 0.13 | 2.77 ± 0.51 | 0.60 ± 0.23 | 2.31 ± 0.37 |
| Gpr31b | protein coding | 0.16 ± 0.10 | 1.36 ± 0.17 | 0.29 ± 0.11 | 1.08 ± 0.23 |
| Gnb1-ps3 | processed | 0.06 ± 0.02 | 1.82 ± 0.53 | 0.20 ± 0.15 | 2.41 ± 0.29 |
| | pseudogene | | | | |
| Ifna12 | protein coding | 0.03 ± 0.05 | 2.68 ± 1.10 | 0.00 ± 0.00 | 2.41 ± 0.27 |
| Gm37711 | TEC | 0.13 ± 0.06 | 1.00 ± 0.33 | 0.32 ± 0.07 | 1.28 ± 0.16 |
| Gm8818 | processed | 0.03 ± 0.06 | 1.51 ± 0.23 | 0.04 ± 0.00 | 0.93 ± 0.33 |
| | pseudogene | | | | |
| Ifna16 | protein coding | 0.00 ± 0.00 | 1.08 ± 0.28 | 0.00 ± 0.00 | 0.95 ± 0.53 |
| Gm13713 | lncRNA | 0.78 ± 0.33 | 9.86 ± 0.65 | 0.03 ± 0.04 | 11.65 ± 1.46 |
| Gm49356 | protein coding | 1.21 ± 0.49 | 7.16 ± 1.89 | 1.64 ± 0.90 | 9.42 ± 2.55 |
| Gm15754 | transcribed | 0.44 ± 0.32 | 3.18 ± 0.95 | 0.18 ± 0.09 | 1.42 ± 0.33 |
| | processed | | | | |
| | pseudogene | | | | |
| Gm28347 | lncRNA | 0.00 ± 0.00 | 3.86 ± 1.27 | 0.00 ± 0.00 | 6.42 ± 0.52 |
| AC147806.1 | protein coding | 0.27 ± 0.26 | 1.99 ± 0.28 | 0.26 ± 0.11 | 2.59 ± 0.90 |
| Gm15056 | protein coding | 0.07 ± 0.13 | 5.07 ± 1.57 | 0.00 ± 0.00 | 8.27 ± 1.94 |
| Gm4761 | processed | 0.14 ± 0.01 | 2.63 ± 1.07 | 0.00 ± 0.00 | 2.37 ± 0.25 |
| | pseudogene | | | | |
| Gm18445 | unprocessed | 0.05 ± 0.05 | 1.75 ± 0.40 | 0.03 ± 0.04 | 1.45 ± 0.45 |
| | pseudogene | | | | |
| Kpna2-ps | processed | 0.28 ± 0.10 | 1.19 ± 0.41 | 0.16 ± 0.10 | 0.98 ± 0.23 |
| | pseudogene | | | | |
| Gm15787 | lncRNA | 0.34 ± 0.32 | 2.36 ± 0.81 | 0.73 ± 0.28 | 1.36 ± 0.11 |
| Ctrl | protein coding | 0.44 ± 0.12 | 2.24 ± 0.78 | 0.12 ± 0.10 | 1.43 ± 0.47 |
| Ppp1r14d | protein coding | 0.51 ± 0.25 | 2.30 ± 0.10 | 0.32 ± 0.14 | 4.12 ± 0.31 |
| Gm7281 | unprocessed | 0.66 ± 0.03 | 3.60 ± 1.53 | 0.33 ± 0.18 | 3.19 ± 0.92 |
| | pseudogene | | | | |
| Olfr760-ps1 | unprocessed | 0.43 ± 0.05 | 2.02 ± 0.17 | 0.70 ± 0.16 | 2.63 ± 0.29 |
| | pseudogene | | | | |
| Gm15856 | lncRNA | 0.21 ± 0.14 | 1.18 ± 0.20 | 0.05 ± 0.03 | 0.82 ± 0.41 |
| Xcl1 | protein coding | 0.05 ± 0.09 | 2.24 ± 0.56 | 0.10 ± 0.14 | 2.13 ± 0.23 |
| 4930445E18Rik | lncRNA | 0.06 ± 0.07 | 1.05 ± 0.32 | 0.11 ± 0.16 | 1.13 ± 0.19 |
| Gm18342 | unprocessed | 0.26 ± 0.12 | 1.74 ± 0.37 | 0.26 ± 0.04 | 2.36 ± 0.30 |
| | pseudogene | | | | |
| Hsf2bp | protein coding | 0.21 ± 0.19 | 1.10 ± 0.30 | 0.02 ± 0.02 | 0.68 ± 0.17 |
| Prm1 | protein coding | 0.13 ± 0.12 | 3.00 ± 0.85 | 0.13 ± 0.01 | 3.58 ± 1.65 |
| AC123856.1 | transcribed | 1.01 ± 0.45 | 15.79 ± 12.18 | 3.38 ± 4.55 | 42.52 ± 4.50 |
| | unprocessed | | | | |
| | pseudogene | | | | |
| Gm44135 | TEC | 0.35 ± 0.48 | 4.47 ± 2.08 | 0.77 ± 0.06 | 4.74 ± 0.38 |
| Gm16026 | transcribed | 0.16 ± 0.16 | 1.23 ± 0.52 | 0.22 ± 0.07 | 1.06 ± 0.18 |
| | unprocessed | | | | |
| | pseudogene | | | | |
| Slc6a19 | protein coding | 0.18 ± 0.22 | 2.19 ± 0.49 | 0.25 ± 0.08 | 2.22 ± 0.28 |

Values indicate mean log-ratio of gene expression in treated cells, quantified in fragments per kilobase of transcript per million mapped reads (FKBP), ± standard deviation; n = 3 (WT + mock treatment; WT + eccDNA); n = 2 (STING KO + eccDNA; Myd88 KO + eccDNA).

### Example 9—Development of an Optimized Method for eccDNA Purification

EccDNAs have been known to occur in almost all cell lines, tissues and organs across species since their first description in 1964¹. Robust eccDNA purification methods are crucial for identifying their genomic origin and understanding their biogenesis. However, their size heterogeneity and low abundance relative to their linear chromosomal counterpart make obtaining high purity eccDNA difficult. Hirt^{11,71}, Alkaline lysis⁷², exonuclease digestion^{16,73,74}, buoyant density^{11,71,73,75}, and combinations of these techniques have been used for eccDNA purification previously. In general, crude DNA circles are extracted from biological samples by Hirt or alkaline lysis, during which large size chromosomal DNAs are co-precipitated and removed with proteins. Then, linear contaminant DNAs within the crude circular DNA are digested with an exonuclease, such as ATP-dependent Plasmid Safe DNase (P.S. DNase)⁷⁴, exonuclease V (RecBCD)¹⁶, or exonuclease III⁷⁶. However, the specificity and efficacy of the exonuclease used must be carefully determined because the low amount of isolated eccDNA may be lost due to trace amounts of contaminating endonucleases or endonuclease activity in the exonuclease. In addition, the exonuclease activity can be inhibited or blocked by certain abnormal and damaged nucleotides⁷⁷, cross linkages⁷⁸, special structures⁷⁹, etc., or their digestion products⁷⁴. Thus, treatment of the crude circular DNA with exonuclease alone is not sufficient for obtaining high purity eccDNA, as demonstrated by a previous study using alkaline lysis and extended exonuclease digestion¹⁰. Although the crude circular DNA from Hirt, alkaline lysis, or even exonuclease treatment could be further purified by cesium chloride-ethidium bromide (CsCl-EB) gradient ultracentrifuge¹¹, given the laborious nature of this technique⁸⁰ it does not satisfy the needs of most studies.

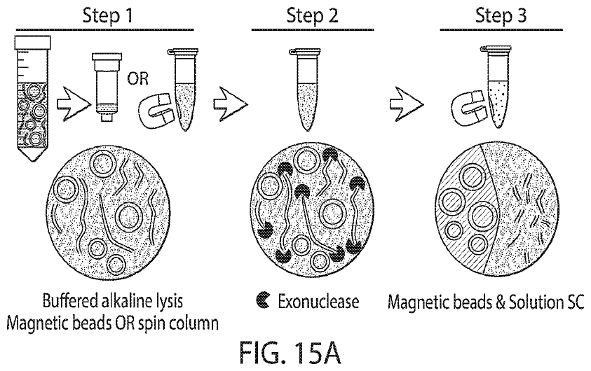

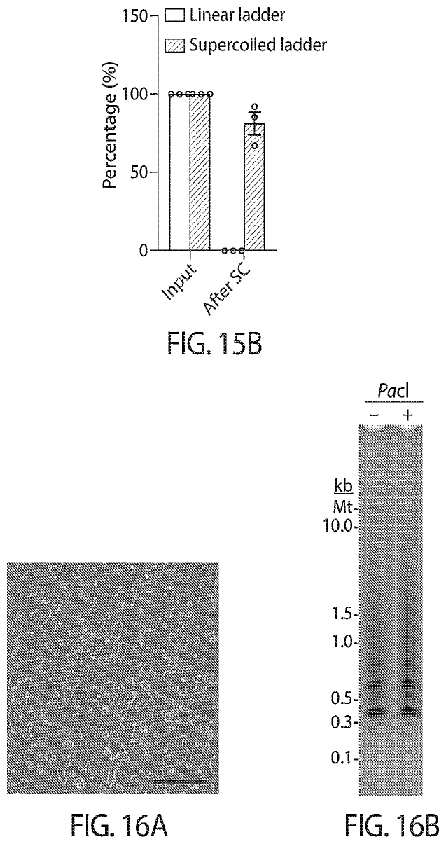

As a robust alternative to the CsCl-EB gradient ultracentrifuge, a robust 3-step eccDNA purification (3SEP) technique is described herein (FIG. 15A), in which reagents are used that do not cause DNA denaturation or have exonuclease activity and are able to selectively bind circular DNA on silica beads while leaving linear DNA in solution, in order to further enrich eccDNA. In the first step, the crude circular DNA is first extracted with buffered (pH 11.8) alkaline lysis, a condition that causes less circular DNA loss than conventional 0.2 M sodium hydroxide¹⁸. In the second step, linear DNA in the crude circular DNA is reduced by the treatment of ATP-dependent Plasmid Safe DNase. Meanwhile, the circular mitochondrial DNA is linearized by restriction digestion with an 8-bp cutter, PacI, which is suitable for eccDNA extraction from whole cells. Finally, a solution is used to recover the circular DNA by its selective binding to magnetic silica beads while leaving any contaminant linear DNA that have escaped exonuclease digestion in the solution (FIGS. 15A and 15B). The 3SEP protocol is designed for eccDNA purification from cultured cells. To adapt the protocol for eccDNA purification from tissue samples, tissue processing such as cell dissociation or tissue grinding should be performed before isolation of the crude circular DNA.

3SEP was developed by further separating circular DNA from linear DNAs that have escaped exonuclease elimination. Compared to previous eccDNA purification methods¹⁰, this method not only results in eccDNA of higher purity, as revealed by microscopy imaging (FIG. 16A), but also takes less time to perform. In contrast to the one week required for previous techniques²⁸¹, the 3SEP procedure can be completed within one day. More importantly, given its high reproducibility, the method can also be used for evaluating the eccDNA yield by mass quantification, visualization in agarose gel (FIG. 16B), or other conventional DNA quantification techniques, with or without further amplification. Moreover, the simplicity of 3SEP allows it to be easily implemented in most conventional molecular biology laboratories. Additionally, since 3SEP DNA recovery uses magnetic separation, an automated version of 3SEP can be developed for diagnostic and clinic applications, particularly for situations related to massive cell death, such as sepsis⁸², cytokine storm⁸³, etc., where rapid isolation and analysis of eccDNA is ideal.

An example 3SEP protocol is provided below, as well as lists of reagents, equipment, and buffers for use during the 3SEP protocol. Although particular reagents and reagent concentrations are provided in the protocol below, it should be understood that in certain instances there may be alternate reagents or reagent concentrations that are also sufficient. Where such alternatives are provided below, these are meant to illustrate without limitation the range of alternatives that are suitable for the described protocol.

### Reagent List:

Methanol, HPLC grade (EMD Millipore, MX0475-1)
Pyrrolidine, 99% (Sigma-Aldrich, P73803-100ML)
Sodium dodecyl sulfate (SDS), 20% solution (Fisher Chemical, BP1311200)
2-Mercaptoethanol, ≥99.0% (Sigma-Aldrich, M7522)
Cetyl trimethylammonium bromide (CTAB) (CAS number: 57090; Sigma, H5882)
Vacuum/spin silica column or magnetic silica beads.
Plasmid-Safe™ ATP-Dependent DNase (Lucigen, E3110k)
Guanidine thiocyanate (CAS number: 593840)
Phenol (CAS number: 108952)
Spermidine (CAS number: 124209)
Hexammine cobalt (III) chloride (CAS number: 10534891)
Qubit™ 1× dsDNA HS Assay Kit (Thermo Scientific, Q33231)
Phase Lock Gel™, QuantaBio, Phase Lock Gel Heavy (VWR, 10847-802)
Phenol/chloroform/isoamyl alcohol, 25:24:1 mixture, pH 8.0 (Thermo Fisher Scientific, BP1752I-400)
Ethyl alcohol, pure, 200 proof, for molecular biology (Sigma-Aldrich, E7023) 1 M Tris, pH 7.0 (Fisher Scientific, AM9851)
UltraPure™ DNase/RNase-free distilled water (Thermo Fisher Scientific, 10977-023)
SYBR™ Gold nucleic acid gel stain, 10,000× concentrate in DMSO (Fisher Scientific, S11494)
Glycogen, MB grade (Sigma-Aldrich, 10901393001)
Dynabeads™ MyOne™ Silane (Thermo Fisher Scientific, 37002D)
PacI (New England Biolabs, R0547)
UltraPure agarose (Thermo Fisher Scientific, 16-500-100)
EDTA (0.5 M), pH 8.0 (Thermo Fisher Scientific, AM9260G)
Ultra-0.5 centrifugal filter unit with Ultracel-10 membrane (Amicon, UFC5010)
5 M NaCl (Thermo Fisher Scientific, AM9760G)
Sodium acetate, 3 M pH 5.5 (Thermo Fisher Scientific, AM9740)

### Equipment List:

Thermal cycler
Benchtop centrifuge
QIAvac 24 Plus vacuum system (Qiagen)
Nanodrop (Thermo Scientific)
Tube Magnet (compatible with 1.5 ml tube)
Qubit Fluorometer (Fisher scientific)
(Optional) NA LoBind tube 1.5 mL, PCR clean (Fisher Scientific, 13-698-791)
(Optional) Mini-PROTEAN® Tetra Vertical Electrophoresis Cell for Mini Precast Gels, 4-gel (Biorad, 1658004)
(Optional) Mini-PROTEAN® Tetra Cell casting module (15-well, 1.5 mm gel) (Biorad, 1658022)

### Reagent Setup:

### Buffer 1

0-100 mM EDTA pH 8.0, preferably 0-20 mM EDTA pH 8.0, or most optimally 10 mM EDTA pH 8.0;
100-200 mM NaCl, or an alternate halide salt (e.g., KCl), most optimally 150 mM NaCl;
0-10% glycerol, preferably 0-5% glycerol, or most optimally 1% glycerol;
Lysis blue (1000×, from Qiagen), or an alternate pH indicator that indicates a change from neutral to basic pH, or vice versa (e.g., pH 7.0 to pH >10.0);
10-300 mg/mL RNase A, preferably 50-150 mg/mL, optimally 110 mg/ml (200× solution);
Optionally, in Tris-HCl, HEPES, or another suitable buffer with a concentration range from 0-500 mM, most preferably 0-20 mM; and
Stored at 4° C. and with 1/500 volume fresh 2-mercaptoethanol or an alternate reducing agent (e.g., DTT, TCEP) added before use.

### Buffer 2

0.01-3.0 M pyrrolidine, or a suitable alternative, such as piperidine or a compound with a pKa between 11.0 and 12.3 (e.g., glucose, arginine, lysine), preferably 0.1-1.5 M pyrrolidine, or most optimally 0.5 M pyrrolidine;
0-100 mM EDTA, preferably 5-30 mM EDTA, or most optimally 20 mM EDTA;
0.1-5% SDS, preferably 0.5-2% SDS, or most optimally 1% SDS
Adjust pH to pH 11.0-12.3, preferably pH 11.3-12.0, and most optimally pH 11.8, with 2 M sodium acetate, pH 4.0, or an alternate acidic solution (e.g., HCl, acetic acid); and
1/500 volume fresh 2-mercaptoethanol or an alternate reducing agent (e.g., DTT, TCEP) added before use.

### Buffer 3

0.5-5.0 M potassium acetate (KOAc) pH 5.5, or an alternate potassium salt, preferably 1.0-3.0 M KOAc pH 5.5, or most optimally 2.0 M KOAc pH 5.5.

### Buffer 4:

0.1%-3% cetyl trimethylammonium bromide (CTAB) in 0.2-3.0 M NaCl.

### Buffer 5

1-50 mM Tris pH 8.0 in 50-90% ethanol.

### Solution CS:

0.2-6.0 M guanidine thiocyanate;
1%-80% phenol;
0.1 μM-500 mM spermidine or spermine; and
0.1 μM-500 mM hexammine cobalt (III) chloride.

### Buffer 6

1.0-4.0 M NaCl with 0-50% ethanol.

### 3SEP Protocol

### Step 1: Crude Circular DNA Isolation

1. (Optional) Fix cells. Wash cells with PBS for 3 times, count cells, resuspend cells with 0.5 ml PBS, then add 9.5 ml absolute methanol, and keep on ice for 10 minutes. Fixed cells can be stored at −20° C. for several months prior to proceeding with circular DNA isolation.
2. Spin down the cells by centrifuging for 2,000×g for 10 minutes at 4° C.
3. Resuspend cells by adding 1 mL Buffer 1 for each 3.5 million cells; scale proportionally to the number of cells. The ratio of Buffer 1 volume to the cell number may vary depending on properties of the cells such as, for example, the cell type.
4. Add an equal volume of Buffer 2 as Buffer 1 used in step 3, then gently mix by inverting tube for 5-10 times. The lysate will become blue, keep 5 minutes at room temperature. As Buffer 2 contains pyrrolidine, the mixture should be handled in a fume hood or under a benchtop exhaust snorkel.
5. Add an equal volume of Buffer 3 as Buffer 1 used in step 3, then gently invert tube until the solution color turns white.
6. Centrifuge the lysate for 10 minutes at 4,500×g and clarified with a filtration cartridge.
7. Estimate the volume of filtrated lysate and add ⅓ of the estimated volume of Buffer 4. Mix by inverting the tube 4-8 times.
8. Add magnetic silica beads or pass the lysate though spin columns, DNAs are bound to either the silica beads or the spin columns.
9. Wash the silica beads or the spin columns with 800 μL Buffer 5.
10. Elute DNAs with water or another suitable low salt buffer, then measure the concentration of circular DNA by either nanodrop or Qubit™ 1× dsDNA HS Assay Kit. The eluted circular DNA can be kept at 4° C. overnight or at −20° C. for long-term storage.

### Step 2: Digest Linear DNA

11. Linearize mitochondrial DNA (mtDNA) using a suitable restriction enzyme (e.g., PacI) and digest linear DNA using ATP-dependent Plasmid Safe DNase, or a suitable alternative. The reaction mixture may contain, for example, 5.0 μL 10×ATP-dependent Plasmid Safe DNase buffer, 2.0 μL 25 mM ATP, 1.0 μL ATP-dependent Plasmid Safe DNase, 1.0 μg to 3.0 μg crude circular DNA, 0.5 μL PacI, 0.25 μL RNase A or RNase T1 (or a suitable alternative), and sterile H₂O up to 50 μL. The reaction mixture may be scaled up, according to the amount of crude circular DNA. Incubate the reaction at 37° C. for 2 hours to overnight.
12. (Optional) Concentrate the reaction mixture. Filtrate the reaction mixture from Step 11 using an Ultra-0.5 Centrifugal Filter Unit (10 kDa MWCO) to produce ˜360 μL of concentrate, according to the manufacturer's instruction.
13. Adjust the solution volume from Step 11 or Step 12 to 360 μL, then extract DNA with phenol/chloroform/isoamyl alcohol (PCI) extraction. Transfer the mixture from Step 11 or concentrate from Step 12 to a Phase Lock Gel tube, add an equal volume of phenol/chloroform/isoamyl alcohol (25:24:1 mixture, pH 8.0), shake the tube by hand thoroughly, and centrifuge at 14,000×g for 7.5 minutes. PCI should be handled in a fume hood or under a benchtop exhaust snorkel.
14. Precipitate the DNA. Transfer the aqueous phase of the PCI extraction to a new tube, add 1/10 volume of sodium acetate (3 M, pH 5.5), add 1 μL glycogen and 3 volumes of 200 proof ethanol, mix, and place the mixture at −80° C. for at least 30 minutes or optionally overnight.
15. Centrifuge the DNA for 30 minutes at 4° C., wash the pellet once with 1 mL 80% ethanol. Spin down the tube, pipet out residual ethanol, resuspend the pellet with 50 μL 2 mM Tris-HCl pH 7.0.
   Step 3: Selectively Recover eccDNA
16. After equilibrating Solution CS at room temperature for 30 minutes, add 700 μL Solution CS to the DNA, and mix the solution by pipet mixing 10 times. Let the solution incubate for 5 minutes at room temperature. Solution CS contains phenol and should be handled in a fume hood or under a benchtop exhaust snorkel.
17. Thoroughly vortex to resuspend the Dynabeads™ MyOne™ Silane beads, take 10 μl to a new tube and place it on a tube magnet for 2 minutes. Remove the liquid from the beads and resuspend in 20 μL Solution CS.
18. Combine the suspended beads with solution from step 16 and pipet mix 10 times. Incubate the mixture for 5 minutes at room temperature.
19. Place the tube on a magnet and allow the beads to settle. Remove and discard the solution from the beads.
20. Spin down the contents of the tube using benchtop centrifuge for ˜10 seconds and place the tube on the magnet again. Remove any residual liquid using a pipet fitted with a 10 μL pipet tip. The eccDNA will be purer as more liquid is removed, but care should be taken to not remove the beads, which will reduce the yield.
21. Remove the tube from the magnet and resuspend the beads in 300 μL Solution CS by pipet mixing. Incubate the mixture for 2 minutes at room temperature.
22. Repeat steps 19-21 twice.
23. With the tube on the magnet, add 800 μL Buffer 6 and incubate for 1 minute without disturbing the beads. Remove the solution and repeat once.
24. With the tube on the magnet, add 800 μL Buffer 5 and incubate for 1 minute without disturbing the beads. Remove the solution and repeat once.
25. Spin down the contents of the tube using a benchtop centrifuge for 30 seconds and place the tube on the magnet. Remove all residual liquid using a pipet fitted with a 10 μl pipet tip, taking caution not to remove beads.
26. Remove the tube from the magnet and thoroughly resuspend the beads with an appropriate volume of 0.1× EB buffer before the beads are completely dry. Rotate the tube for at least 3 minutes.
27. Place the tube on the magnet to settle the beads. Transfer the eluate to a new DNA LoBind Tube.
28. Measure the eccDNA concentration by Quibit, according to the manufacturer's instruction.

### (Optional) Vertical Agarose Gel Electrophoresis

29. Mix 1.0 g ultra-pure agarose with 100 mL 1× Tris-acetate EDTA buffer (TAE) in a microwave flask. Microwave for 3-5 minutes until the agarose is completely dissolved. The gel concentration may range from 1-2%.
30. Pour the agarose into pre-assembled glass plates immediately after the agarose is dissolved. Insert the well comb and allow the gel to cool for at least 20 minutes.
31. Remove the comb after dismounting the gel from casting chamber. Remove any residual gel slices within wells using a sharp tweezer.
32. Assemble the gel in the gel apparatus according to the manufacturer's instructions, then fill the chamber with 1×TAE.
33. Carefully pipet eccDNA samples (e.g., >1 ng eccDNA) into wells, alongside a suitable amount of DNA ladder.
34. Separate the DNA at 80 V for 35 minutes. The running time may differ depending on the gel concentration and apparatus manufacturer.
35. Turn the power off, open the glass plates and transfer the gel to a dish. Add 50-70 mL 1×TAE.
36. Add 5 μL Sybr Gold concentrate and shake the gel for at least 15 minutes in the dark.
37. Visualize eccDNA in the gel using any suitable device that emits blue light.

### (Optional) Scan Atomic Force Microscopy Imaging

38. Take 4.5 μL eccDNA with a concentration between 0.6 ng/μl and 1.0 ng/μl, add 0.5 μl ( 1/10 volume) 10× imaging buffer, and mix by pipetting.
39. Cleave a mica surface (e.g., using double side tape). Spread the DNA mixture on the mica surface and incubate for 2 min.
40. Blow off the liquid using compressed gas and rinse the mica twice with 30 μL of 2.0 mM magnesium acetate. Repeat once.
41. Image the eccDNA by using tip C of an SNL-10 probe, scanning with Air mode, and processing with Gwyddion (e.g., using a Veeco MultiMode atomic-force microscope with a Nanoscope V Controller in ‘ScanAsyst in Air mode’).

### Methods

### Cell Cultures and Apoptosis Induction

Mouse ESC-E14 cells were cultured on dishes coated with 0.1% gelatin in standard LIF/serum medium containing mouse LIF (1000 U/mL), 15% fetal bovine serum (FBS), 0.1 mM nonessential amino acids, 0.055 mM β-mercaptoethanol (BME), 2 mM GlutaMax, 1 mM sodium pyruvate and penicillin-streptomycin (PS). HeLa S3 cells were grown in Dulbecco's modified Eagle's medium (DMEM) with 10% FBS and 100 U/mL PS. CH12F3 cells were cultured in RPMI1640 with 10% heat inactivated FBS, 100 U/mL PS, 2 mM BME, 2 mM GlutaMax. L929 cells were cultured in DMEM supplemented with 10% heat inactivated FBS and 100 U/mL PS. Apoptotic cell death of mESC were induced with 0.5 μM Etoposide (Selleck), 2 μM Staurosporine (Selleck) for 24 hours, or irradiated without medium using ultraviolet (UV-C) in Stratagene Stratalinker 2400 for 3 mJ and continue culture for 16 hours. CH12F3 apoptotic cell death were treated with 2 μM Staurosporine for 16 hours. Cell viability was analyzed with BD FACSCanto II after staining with Live/Dead Fixable Far Red Dead Cell Stain Kit.

### Knockout Cell Line Generation

DNase 7 and EndoG knockout mESC cell lines were generated by CRISPR/Cas9 with transit transfection of px330-mCherry (Addgene 98750). mCherry positive cells were sorted by flow-cell cytometry. Guide RNA targeting sequences and PCR genotyping primers for DNase 7 and EndoG were listed in Table 4. Lig1−/− CH12F3 cell line was generated by CRISPR/Gas9 guide RNAs targeting to intron 17 and 19 to delete exon 18-19, which harbors the conserved ligase catalytic site. The deletion resulted in a premature stop codon. NucLig3−/−CH12F3 cell line was generated by specifically deleting sequences containing the nucLig3 start codon and the two subsequent Methionines (Met89-Met144) with CRISPR/Cas9 guide RNAs, while keeping the mtLig3 in frame and functional. Lig4 is a single exon gene, two pairs of guide RNAs were used for two rounds of targeting to obtain homozygous deletion of the entire 2.7 kb exon. Guide RNA sequences were list in Table 4. Knockout cell lines were confirmed by immunoblotting.

TABLE 4
Guide RNA targeting sequences and PCR primers
for DNase γ and Endonuclease G

| | |
|---|---|
| Sequence | SEQ ID |

mESC/E14 cells

| | | | |
|---|---|---|---|
| | Guide RNA | | |
| DNase γ | DNase γ-guide1_Fw | CACCGCAGAACATTCGTTTGAGGA | 1 |
| | DNase γ-guide1_Rv | AAACTCCTCAAACGAATGTTCTGC | 2 |
| | DNase γ-guide2_Fw | CACCGCATGGTCTCACCAGTAAAGG | 3 |
| | DNase γ-guide2_Rv | AAACCCTTTACTGGTGAGACCATGC | 4 |
| | DNase γ-guide3_Fw | CACCGTTGGGGATTGAAACGCGTGA | 5 |
| | DNase γ-guide3_Rv | AAACTCACGCGTTTCAATCCCCAAC | 6 |
| Endonuclease G | EndoG-guide1_Fw | CACCGCCGAGTGCCATTGTTGCCGG | 7 |
| | EndoG-guide1_Rv | AAACCCGGCAACAATGGCACTCGGC | 8 |
| | EndoG-guide2_Fw | CACCGTGGTAAGGCAGGTCAGACGT | 9 |
| | EndoG-guide2_Rv | AAACACGTCTGACCTGCCTTACCAC | 10 |
| | PCR genotγping | | |
| DNase γ | DNase γ-neg_Fw | GTGGGTGTGGAGGCTCTTTT | 11 |
| | DNase γ-neg_Rv | GATTCTGGCCTGTAGGCACT | 12 |
| | DNase γ-pos_Fw | AAGGGAGCTGACTCCTTCTCA | 13 |
| Endonuclease G | EndoG-neg_Fw | ACCCCACACCTTTTGTTGCT | 14 |
| | EndoG-neg_Rv | CACACCTCGGACCTTCCATT | 15 |
| | EndoG-pos_Fw | GTGGAACGACCAATGGCAAG | 16 |
| | EndoG-Pos_Rv | TAACAAGCACGAGTGGGAGG | 17 |

CH12F3 cells

| | | | |
|---|---|---|---|
| | Guide RNA | | |
| Lig1 | Lig1-In17.1_Fw | CACCGCTTCTGCTGACATAAGGGAG | 18 |
| | Lig1-In17.1_Rv | AAACCTCCCTTATGTCAGCAGAAGC | 19 |
| | Lig1-In19.1_Fw | CACCGGACCACCCTGATTCTGTGG | 20 |
| | Lig1-In19.1_Rv | AAACCCACAGAATCAGGGTGGTCC | 21 |
| NucLig3 | Lig3-3Mdel-1_Fw | CACCGCCGCTGCTCTGCCATCGCAC | 22 |
| | Lig3-3Mdel-1_Rv | AAACGTGCGATGGCAGAGCAGCGGC | 23 |
| | Lig3-3Mdel-2_Fw | CACCGTGAGAAACTGGAACGGGCTC | 24 |
| | Lig3-3Mdel-2_Rv | AAACGAGCCCGTTCCAGTTTCTCAC | 25 |
| | Round 1 | | |
| | targeting | | |
| | guide RNA | | |
| Lig4 | Lig4-1.2_Fw | CACCGTGGAACATAAGTCTGCAAA | 26 |
| | Lig4-1.2_Rv | AAACTTTGCAGACTTATGTTCCAC | 27 |
| | Lig4-2.1_Fw | CACCGGTGTCCGATTCAGTAGACA | 28 |
| | Lig4-2.1_Rv | AAACTGTCTACTGAATCGGACACC | 29 |
| | Round 2 | | |
| | targeting | | |
| | guide RNA | | |
| | Lig4-1.1_Fw | CACCGCTTTATCAGTTCAAACCGG | 30 |
| | Lig4-1.1_Rv | AAACCCGGTTTGAACTGATAAAGC | 31 |
| | Lig4-2.2_Fw | CACCGTATTTGCTTTAGAGCTTGC | 32 |
| | Lig4-2.2_Rv | AAACGCAAGCTCTAAAGCAAATAC | 33 |

### EccDNA Purification

To purify eccDNAs used in Examples 1-8, cells were first dehydrated in >90% methanol before crude extrachromosomal DNA were extracted in an alkaline lysis buffer at pH 11.8. After neutralization and precipitation, crude extrachromosomal DNA were bound in silica column (QIAGEN Plasmid Plus Midi Kit) in binding buffer (Buffer BB of QIAGEN Plasmid Plus Midi Kit). Bound DNAs were eluted and digested with PadI (NEB) and Plasmid Safe ATP-dependent DNase (Lucigen) for 4-16 hours. Undigested eccDNA is then extracted with Phenol/chloroform/isoamyl alcohol (PCI) solution (25:24:1) in a Phase Lock Gel tube (QuantaBio) to minimize DNA loss. After precipitation with carrier glycogen (Roche) and 1/10 V 3 M NaOAc (pH 5.5), the precipitated crude eccDNAs were resuspended in Solution A (One-Step Max Plasmid DNAout, TIANDZ) and the eccDNAs can selectively bound to magnetic silica beads in this solution. The eccDNAs were then eluted with 0.1× Elution buffer (1 mM Tris-HCl pH 8.0) and the concentration is measured by Qubit dsDNA HS Assay kit (Thermo Fisher).

An optimized method for eccDNA purification is described in Example 9.

### Synthetic Small DNA Circle Preparation

Synthetic small DNA circles were prepared by the procedure of Ligase Assisted Minicircle Accumulation (LAMA). Random DNA sequence were generated at (https://faculty.ucr.edu/˜mmaduro/random.htm) with 50% GC content. The isomers of single strand template as well as their amplification primer sets were synthesized from IDT and their sequences are listed in Table 5. Products with 5′end phosphate were prepared with 2×Q5 DNA polymerase mixtures (NEB). Equal amount of isomers were added to make 100 μL HiFi Taq DNA ligase reaction mixtures, and placed in thermo cyclers following cycles: 95° C. for 3 min, 60° C. for 10 min and 37° C. for 5 min for at least 10 cycles. Circularized products were recovered by PCR Purification Kit (Qiagen) and digested with Plasmid Safe ATP-dependent DNase (Lucigen) before being recovered with PCR Purification Kit.

TABLE 5
Synthetic small DNA and its PCR amplification
primer sequences

| | | Sequence: | SEQ ID: |
|---|---|---|---|
| | Template for | | |
| | artificial | | |
| | DNA circle: | | |
| Circle 2 | R2 | CATCCTCTCACAGCACCATGCAGGCCGGCGTA | 34 |
| (C2): 200 bp | | CGGGTCCCATATAAACCTGTCATAGCTTACCT | |
| synthesized | | GACTCTACTTGGAAATGTGGCTAGGTCTTTGC | |
| DNA circle | | CCACGCACCTAATCGGTCCTCGTTTGCTAGGC | |
| | | CTGACCCGATGAACTACAGAACACTGCAAGAA | |
| | | TCTCTACCTGCTTTACAAAGTGCTGGATCCTA | |
| | | TTCCAGCG | |
| | F2 | CGCACCTAATCGGTCCTCGTTTGCTAGGCCTG | 35 |
| | | ACCCGATGAACTACAGAACACTGCAAGAATCT | |
| | | CTACCTGCTTTACAAAGTGCTGGATCCTATTC | |
| | | CAGCGCATCCTCTCACAGCACCATGCAGGCCG | |
| | | GCGTACGGGTCCCATATAAACCTGTCATAGCT | |
| | | TACCTGACTCTACTTGGAAATGTGGCTAGGTC | |
| | | TTTGCCCA | |
| | PCR primer | | |
| | sets to | | |
| | amplify | | |
| | template: | | |
| | R2-Fw | p-CATCCTCTCACAGCACCATGC | 36 |
| | R2-Rv | p-CGCTGGAATAGGATCCAGCAC | 37 |
| | F2-Fw | p-TGGGCAAAGACCTAGCCACATT | 38 |
| | F2-Rv | p-CGCACCTAATCGGTCCTCG | 39 |
| | Template for | | |
| | artificial | | |
| | DNA circle: | | |
| Circle 5 | F5 | GACTCACAGGAAAGGTGCGTATAGAGCCCAGC | 40 |
| (C5): 525 bp | | GCAAAGAGGGAGAGCTCAAATGATGTTACTTA | |
| synthesized | | GAGAACCTCAGTAATGATTTCACGCGGGGAAA | |
| | | GCTCAAACAAGTGCTATACATGGATTACTCGG | |
| | | TGACTTCGATGGAAATCACCCGTGTGTGGGTA | |
| | | CCGCGTATAATCGCGTTCCTTGTAATGAAGTA | |
| | | TGTTGCCGTTTCATGTGTACAGCATTATGGTC | |
| | | AACGAAGCATTGCTCTCTTGAACAGAAGCTAT | |
| | | TGGTCCACTTCATGTGCATAAAGATAAGTATC | |
| | | ATACCCGCACTTTACATAGGATAGTTTAATCT | |
| | | TGCTTTGCAATAGAACGGTTCTTCGGCTTGGG | |
| | | AGGGTACAAAGCCGATGAGACTCACACTTCCC | |
| | | CGTACTTACCTTCGTTCGTTGGCTCGACTTAC | |
| | | CGATAAATACGCTAGAGACGTCTAGACCCAGT | |
| | | GGTGTTTACCTATAAGACCATATTCAGTGCAG | |
| | | CTTTCATAGAATCGGCCTCATATGTGCTTAGG | |
| | | TCTACCCGATTTG | |
| DNA circle | R5 | TATTGGTCCACTTCATGTGCATAAAGATAAGT | 41 |
| | | ATCATACCCGCACTTTACATAGGATAGTTTAA | |
| | | TCTTGCTTTGCAATAGAACGGTTCTTCGGCTT | |
| | | GGGAGGGTACAAAGCCGATGAGACTCACACTT | |
| | | CCCCGTACTTACCTTCGTTCGTTGGCTCGACT | |
| | | TACCGATAAATACGCTAGAGACGTCTAGACCC | |
| | | AGTGGTGTTTACCTATAAGACCATATTCAGTG | |
| | | CAGCTTTCATAGAATCGGCCTCATATGTGCTT | |
| | | AGGTCTACCCGATTTGGACTCACAGGAAAGGT | |
| | | GCGTATAGAGCCCAGCGCAAAGAGGGAGAGCT | |
| | | CAAATGATGTTACTTAGAGAACCTCAGTAATG | |
| | | ATTTCACGCGGGGAAAGCTCAAACAAGTGCTA | |
| | | TACATGGATTACTCGGTGACTTCGATGGAAAT | |
| | | CACCCGTGTGTGGGTACCGCGTATAATCGCGT | |
| | | TCCTTGTAATGAAGTATGTTGCCGTTTCATGT | |
| | | GTACAGCATTATGGTCAACGAAGCATTGCTCT | |
| | | CTTGAACAGAAGC | |
| | PCR primer | | |
| | sets to | | |
| | amplify | | |
| | template: | | |
| | F5-Fw | p-GACTCACAGGAAAGGTGCGT | 42 |
| | F5-Rv | p-CAAATCGGGTAGACCTAAGCACA | 43 |
| | R5-Fw | p-TATTGGTCCACTTCATGTGC | 44 |
| | R5-Rv | p-GCTTCTGTTCAAGAGAGCAA | 45 |

### Scanning Atomic Force Microscope (SAFM) Imaging

SAFM imaging of DNA was performed in dry mode19. Briefly, one tenth volume of 10× imaging buffer (100 mM NiCl2 and 100 mM Tris-HCl, pH8.0) was added to sample to reach final DNA concentration of 0.6-1.0 ng/μL, then 2-5 μL mixture was spread on freshly cleaved mica (Ted Pella) surface. After 2 minutes of incubation, specimen was rinsed twice with 30 μL 2 mM Mg(OAc)2, and specimen was dried before and after rinse with compressed air. Images were taken by using tip C of SNL-10 probe on a Veeco MultiMode AFM with Nanoscope V Controller in “ScanAsyst in Air mode” and processed with Gwyddion 2.57.

Library Preparation and eccDNA Sequencing

Nanopore sequencing library for eccDNA was prepared by following Ligation Sequencing Kit (Oxford Nanopore) according to manufactory's instruction after rolling cycle amplification and debranching. RCA were performed with phi29 DNA polymerase (NEB) with some modification to ensure efficient amplification from 100 μg template per reaction. Briefly, in a 20 μL reaction mixture: 2 μL 10× phi29 DNA polymerase buffer (NEB), 2 μL 25 mM dNTPs, 1 μL Exo Resistant Random Primer (Thermo Fisher), and eccDNA ≥100 μg, were added with ultra-pure H₂O to 17.6 μL, mixed and incubated at 95° C. for 5 minutes before ramping to 30° C. at 1% Ramp Rate. Then, 1 μL phi29 DNA polymerase, 0.6 μL Pyrophosphatase Inorganic (yeast, NEB), 0.4 μL 0.1M DTT (NEB), 0.4 μL 20 mg/mL BSA (NEB) were added. The reaction mixture was incubated at 30° C. for 10-16 hours. Since high branch structure of RCA products could block nanopore to abolish sequencing, RCA products of eccDNAs were further debranched with T7 Endonuclease I (NEB) before being used for sequencing library construction with Ligation Sequencing Kit (Oxford Nanopore, SQK-LSK109). The library was sequenced in Flow cell (R9.4.1, FL-MIN106D) on MinION according to manufactory's instruction.

Illumina sequencing library for eccDNA was prepared by Tn5-transposon-based tagmentation with Nextera® XT DNA Sample Preparation Kit according to manufactory's instruction. Briefly, after validating the purity of eccDNAs with SAFM imaging, 0.5 ng pure eccDNAs were directly tagmented with Tn5 transposase, followed by 12-14 cycles PCR amplification with Illumina sequencing adaptors. Barcoded libraries were pooled and sequenced with Illumina 2500 in 150 PE mode.

### EccDNA Sequencing Data Analyses

Nanopore base calling and reads mapping. The fast5 files generated by Nanopore MinION were fed to Guppy (version 3.5.2) for base calling. The parameters used for Guppy were: --flowcell FLO-MIN106 --kit SQK-LSK109 --qscore_filtering --calib_detect --trim_barcodes --trim_strategy dna --disable_pings --device auto --num_callers 16. The generated reads in fastq format were further processed by porechop (version 0.2.4) to remove adaptor sequences for each read with parameters: --extra_end_trim 0 --discard_middle. To reduce artifacts due to misalignment during reads mapping, a customized reference mouse genome (mm10combine) was compiled based on mm10 reference sequences. Then the cleaned reads were aligned to mm10combine using minimap2⁶¹ (version 2.17) with parameters: -x map-ont -c --secondary=no -t 16. The alignments for each read were stored in PAF format.

Consensus eccDNA Generation

To obtain the consensus boundary and sequence of each eccDNA from the mapped RCA long reads, a tool was developed (https://github.com/YiZhang-lab/eccDNA_RCA_nanopore) that uses the alignments in PAF file as input and outputs eccDNA fragments composition (chromosome, genomic start and end positions of each fragment), successive fragments coverage (number of passes) and consensus sequence derived from each RCA long read. The sub-reads of each RCA long read could be mapped to one genomic location or multiple locations. The sub-reads with mapping quality lower than 30 were discarded. This tool performed bootstrapping of successive sub-reads in each RCA long read to check whether the order of the mapped genomic locations for each sub-read is concordant with their order in the RCA long read. Due to the inaccuracy and gap-prone property of Nanopore reads, a maximum of 20 bp offset of the mapped genomic positions was allowed (start and end position, respectively) between two sub-reads to be considered as mapping to the same location. Reads with discordant sub-reads order, location or strand would be discarded. The exact boundaries of eccDNA fragments are determined by voting from the sub-reads' start and end positions, respectively. The boundary positions were further refined by threading the sub-reads to ensure no gaps or overlaps between any successive sub-reads. The number of passes for each eccDNA fragment was calculated as the number of concordant sub-reads mapped to that location. Only eccDNA with at least 2 passes was kept for downstream analysis. Each eccDNA sequence was derived from the reference genome sequence where it mapped to, with sequence variants incorporated. The sequence variants were called from sub-reads mapped to the corresponding location, requiring minimum depth of 4 and minimum allele frequency of 0.75.

Genomic Distribution of eccDNA

The eccDNA fragments were piled up across the genome. To remove PCR duplicates, eccDNA fragments with the same chromosome, start and end positions were treated as duplicates and only one was retained. The coverage of unique eccDNA fragments at each base of the genome was obtained using bedtools62 (version 2.29.2) and stored in bigwig file. The distribution of eccDNA across each chromosome was plotted using karyoploteR63 (version 1.14.1) with the bigwig fie as input.

### Mapping of Illumina Sequencing Reads

Raw Illumina sequence reads were first processed by Trimmomatic64 (version 0.39) to remove sequencing adaptors and low-quality reads, using parameters: ILLUMINACLIP:adapters/NexteraPE-PE.fa:2:30:10:1:true LEADING:3 TRAILING:3 SLIDINGWINDOW:4:15 MINLEN:75 TOPHRED33. BWA65 MEM (version 0.7.17) with default parameters. Then, the reads were mapped to the customized mm10combine reference genome. Duplicated reads were removed by Picard (version 2.23.4). Reads with mapping quality of at least 60 were considered as uniquely mapped and used for downstream analysis. The genomic coverage was calculated using bamCoverage from deeptools66 (version 3.5.0) with binSize 1.

### Western Blot Analysis

Equal numbers of cells were lysed in NuPAGE LDS Sample buffer (Thermo Fisher) and protein extracts were resolved on SDS-PAGE and transferred to PVDF membrane. Antibodies against Lig1 (Proteintech), Lig3 (BD Biosciences), Lig4 (a gift from David Schatz at Yale), Myd88(ProSci), Sting (Proteintech), and Gapdh (Thermo Fisher) were used.

### Bone Marrow Derived Dendritic Cells and Macrophages Preparation and Stimulation

8-12 weeks old male mice were used for preparing bone marrow cells (BMs) and all mice were from Jackson lab., including WT C57/BL6, Sting−/− (Tmem173gt, Stock No: 017537), and Myd88−/− (Myd88tm1.1Defr, Stock No: 009088). BMDC were differentiated in RPMI1640 medium supplemented with 10% heat inactivated FBS (Sigma), 10 mM HEPES, 1 mM sodium pyruvate, 100 U/mL PS, 2 mM GlutaMax and 20 ng/mL mouse GM-CSF (Peprotech). BMDM were differentiated in DMEM supplemented with 10% heat inactivated FBS, 100 U/mL PS, and 20% L929 conditioned medium. Half of the medium was replaced at day 3 and day 6. Identity of BMDCs were confirmed with CD11c+ and MHC II+ double positivity. Identity of BMDMs were confirmed with F4/80+ and CD11b+ double positivity. For cell stimulation, cells at day 7-9 were seeded in 96 wells-plates at 3.5×10⁴ per well. DNA was transfected to cells with FuGENE HD (Promega) in Opti-MEM (Gibco) according to manufactory's instructions after measuring their concentrations with Qubit dsDNA HS Assay kit (Thermo Fisher). After 12 hours' transfection, media were collected for ELISA, and cells were lysed with Trizol (Thermo Fisher) for RNA isolation.

### Transfection Efficiency Assay

To determine the transfection efficiency of linear DNA and circular DNA, a set of primers that contained 5 Phosphorothioate bonds at their 5′end was used to prepare ends protected linear DNA by PCR (see Table 6 for sequences). To balance the effects of Phosphorothioate bonds, the circular form was prepared with the same number of Phosphorothioate bonds as linear one. Then DNA concentration was determined by Qubit dsDNA HS Assay kit (Thermo Fisher), then transfected to BMDCs as described above with FuGENE HD (Promega). After transfection, cells were rinsed with PBS for 3 times, and lysed in 100 μL lysis buffer [50 mM Tris-HCl pH 8.0, 1 mM EDTA, 0.5% Tween-20, 3 units/mL Thermoliable Proteinase K (NEB, Cat #P811IS)], then incubate at 37° C. for 2 hour and followed with 15 minutes incubation at 55° C. to inactivate Proteinase K. 4 μL cell lysate were used for qPCR with a set of primers targeting to both linear and circular DNA to determine the transfected DNA level.

Incubation BMDC with Apoptotic Medium

Apoptotic medium was prepared by UV irradiation. 80% confluent wildtype and DNase 7 −/− cells in 10 cm dishes were washed 3 times with PBS. After irradiating cells with 3 mJ of ultraviolet (UV-C) in Stratagene Stratalinker 2400, 10 mL Opti-MEM (Gibico) were added and cultured for another 48 hours. Collected medium was centrifuged at 650 g for 5 minutes and supernatant was filtrated with 0.45 m filters. After treatment with enzymes at 37° C. for 2 hours, the supernatant was dialyzed (MWCO, 10 kDa) with fresh Opti-MEM at 4° C. for overnight to deplete ATP that is required for the activity of Plasmid Safe ATP-dependent DNase. Then, BMDCs in 96 wells plate were added with 100 μL of dialyzed medium per well. After 12 hours of incubation, cells were collected for RNA isolation and RT-qPCR analysis.

### RNA Isolation, RT-qPCR, RNA-Seq and ELISA Analyses

Cellular RNA was isolated with Zymo Direct-zol RNA Miniprep kit. Complementary DNA was synthesized with SuperScript III and qPCR was performed with Fast SYBR Green Master Mix (Thermo Fisher). The primer sequences for qPCR of each genes are listed in Table 6. Relative gene expression level was calculated after normalizing to Gapdh. Bulk RNA-seq libraries were prepared by following the NEBNext Ultra Directional RNA Library Prep Kit for Illumina (NEB, catalog no. E7420S). For ELISA analysis, ELISA kits of IFN-β, IL-6 and TNF-α were obtained from Biolegend, IFN-α ELISA kits were from PBL Assay Science, assays were performed according to manufactory's instructions. Appropriate volumes of culture medium were used to ensure that the readouts are within the range of the standard curve.

TABLE 6
Real time quantitative PCR (RT-qPCR) primers

| | RT-QPCR | | SEQ |
|---|---|---|---|
| Target: | primer: | Sequence: | ID: |
| GAPDH | mGAPDH-Fw | AGGTCGGTGTGAACGGATTTG | 46 |
| | mGAPDH-Rv | TGTAGACCATGTAGTTGAGGTCA | 47 |
| IFNα4 | IFNα4-Fw | TGATGGTCTTGGTGGTGAT | 48 |
| | IFNα4-Rv | TTGTGCCAGGAGTGTCAA | 49 |
| IFN-β | IFN-β-Fw | GGTGGAATGAGACTATTGTTG | 50 |
| | IFN-β-Rv | CTTCAAGTGGAGAGCAGTT | 51 |
| IL-1β | IL-1β-Fw | TGCCACCTTTTGACAGTGATG | 52 |
| | IL-1β-Rv | TGTGCTGCTGCGAGATTTGA | 53 |
| IL-6 | IL-6-Fw | CGGCCTTCCCTACTTCACAA | 54 |
| | IL-6-Rv | TTGCCATTGCACAACTCTTTTC | 55 |
| TNF-α | TNF-α-Fw | GTCCCCAAAGGGATGAGAAGT | 56 |
| | TNF-α-Rv | TTTGCTACGACGTGGGCTAC | 57 |

### RNA-seq Data Analysis

For RNA-seq data, adaptors and low-quality reads were trimmed using Trimmomatic⁶⁴ (version 0.39) with parameters: ILLUMINACLIP:adapters/TruSeq3-PE.fa:2:30:10:1:true LEADING:3 TRAILING:3 SLIDINGWINDOW:4:15 MINLEN:50 TOPHRED33. The cleaned paired-end reads were aligned to mm10 reference genome with GENCODE⁶⁷ mouse gene set M24, using STAR⁶⁸ (version 2.7.6a) with parameters: --outSAMunmapped Within --outFilterType BySJout --outSAMattributes NH HI AS NM MD --outFilterMultimapNmax 20 --outFilterMismatchNmax 999 --outFilterMismatchNoverReadLmax 0.04 --alignIntronMin 20 --alignIntronMax 1000000 --alignMatesGapMax 1000000 --alignSJoverhangMin 8 --alignSJDBoverhangMin 1 --sjdbScore 1 --outSAMtype BAM SortedByCoordinate --quantMode TranscriptomeSAM. RSEM⁶⁹ (version 1.3.3) was used to quantify gene expression levels using the reads aligned to transcriptome in bam file as input, with parameters: --alignments --estimate-rspd --calc-ci --no-bam-output --seed 12345 --ci-memory 30000 --paired-end --strandedness reverse. The differential expressed genes were identified using DESeq2 package⁷⁰.

eccDNA Linearization

EccDNA linearization was performed by sequential treatment of eccDNAs with the nickase fnCpf1³⁷ (Applied Biological Materials) and single strand DNA-specific nuclease. 50 ng eccDNAs were nicked in a 20 μL reaction that contained ⅛ volume of 8×fnCpf1 linearization buffer (160 mM HEPES pH7.5, 1.2 M KCl, 4 mM DTT, 0.8 M EDTA, 80 mM MnCl2) and 1 μL fnCpf1. After incubating at 37° C. for 1 hour, the treated eccDNAs were extracted with Phenol/chloroform/isoamyl alcohol (PCI) solution (25:24:1) in a Phase Lock Gel tube (QuantaBio) and precipitated at −80° C. with carrier glycogen (Roche) and 1/10 V 3M NaOAc (pH 5.5). Nicked eccDNAs were linearized in 10 μL reaction that include 2 μL 5× buffer (0.25 M NaOAc pH 5.2, 1.4 M NaCl, 25 mM ZnSO4), 1 μL S1 Nuclease (Thermo Fisher) at 37° C. for 5 min. The reaction was stopped by adding 40 μL 10 mM Tris-HCl (pH 8.0), and the linear eccDNA were immediately recovered by 75 μL SPRIselect beads (Beckman Coulter). Successful linearization of eccDNAs was confirmed by efficient digestion with Plasmid Safe ATP-dependent DNase (Lucigen).

### REFERENCES

1. Hotta, Y. & Bassel, A. Molecular Size and Circularity of DNA in Cells of Mammals and Higher Plants. Proc Natl Acad Sci USA 53, 356-362, doi:10.1073/pnas.53.2.356 (1965).
2. Moller, H. D. et al. Circular DNA elements of chromosomal origin are common in healthy human somatic tissue. Nat Commun 9, 1069, doi:10.1038/s41467-018-03369-8 (2018).
3. Horowitz, H. & Haber, J. E. Identification of autonomously replicating circular subtelomeric Y′ elements in Saccharomyces cerevisiae. Mol Cell Biol 5, 2369-2380, doi:10.1128/mcb.5.9.2369 (1985).
4. Sinclair, D. A. & Guarente, L. Extrachromosomal rDNA circles—a cause of aging in yeast. Cell 91, 1033-1042, doi:10.1016/s0092-8674(00)80493-6 (1997).
5. Paulsen, T., Kumar, P., Koseoglu, M. M. & Dutta, A. Discoveries of Extrachromosomal Circles of DNA in Normal and Tumor Cells. Trends Genet 34, 270-278, doi:10.1016/j.tig.2017.12.010 (2018).
6. Gaubatz, J. W. Extrachromosomal circular DNAs and genomic sequence plasticity in eukaryotic cells. Mutat Res 237, 271-292, doi:10.1016/0921-8734(90)90009-g (1990).
7. Cox, D., Yuncken, C. & Spriggs, A. I. Minute Chromatin Bodies in Malignant Tumours of Childhood. Lancet 1, 55-58, doi:10.1016/s0140-6736(65)90131-5 (1965).
8. Turner, K. M. et al. Extrachromosomal oncogene amplification drives tumour evolution and genetic heterogeneity. Nature 543, 122-125, doi:10.1038/nature21356 (2017).
9. Shibata, Y. et al. Extrachromosomal microDNAs and chromosomal microdeletions in normal tissues. Science 336, 82-86, doi:10.1126/science.1213307 (2012).
10. Dillon, L. W. et al. Production of Extrachromosomal MicroDNAs Is Linked to Mismatch Repair Pathways and Transcriptional Activity. Cell Rep 11, 1749-1759, doi:10.1016/j.celrep.2015.05.020 (2015).
11. Smith, C. A. & Vinograd, J. Small polydisperse circular DNA of HeLa cells. J Mol Biol 69, 163-178, doi:10.1016/0022-2836(72)90222-7 (1972).
12. Hull, R. M. et al. Transcription-induced formation of extrachromosomal DNA during yeast ageing. PLoS Biol 17, e3000471, doi:10.1371/journal.pbio.3000471 (2019).
13. Gresham, D. et al. Adaptation to diverse nitrogen-limited environments by deletion or extrachromosomal element formation of the GAP1 locus. Proc Natl Acad Sci USA 107, 18551-18556, doi:10.1073/pnas.1014023107 (2010).
14. Verhaak, R. G. W., Bafna, V. & Mischel, P. S. Extrachromosomal oncogene amplification in tumour pathogenesis and evolution. Nat Rev Cancer 19, 283-288, doi:10.1038/s41568-019-0128-6 (2019).
15. DeLap, R. J., Rush, M. G., Zouzias, D. & Khan, S. Isolation and preliminary characterization of the small circular DNA present in African green monkey kidney (BSC-1) cells. Plasmid 1, 508-521, doi:10.1016/0147-619x(78)90008-2 (1978).
16. Shoura, M. J. et al. Intricate and Cell Type-Specific Populations of Endogenous Circular DNA (eccDNA) in Caenorhabditis elegans and Homo sapiens. G3 (Bethesda) 7, 3295-3303, doi:10.1534/g3.117.300141 (2017).
17. Zhu, J. et al. Molecular characterization of cell-free eccDNAs in human plasma. Sci Rep 7, 10968, doi:10.1038/s41598-017-11368-w (2017).
18. Cloninger, C., Felton, M., Paul, B., Hirakawa, Y. & Metzenberg, S. Control of pH during plasmid preparation by alkaline lysis of Escherichia coli. Anal Biochem 378, 224-225, doi:10.1016/j.ab.2008.04.017 (2008).
19. Liu, D. et al. Branched kissing loops for the construction of diverse RNA homooligomeric nanostructures. Nat Chem 12, 249-259, doi:10.1038/s41557-019-0406-7 (2020).
20. Landry, J. J. et al. The genomic and transcriptomic landscape of a HeLa cell line. G3 (Bethesda) 3, 1213-1224, doi:10.1534/g3.113.005777 (2013).
21. Tichy, E. D. Mechanisms maintaining genomic integrity in embryonic stem cells and induced pluripotent stem cells. Exp Biol Med (Maywood) 236, 987-996, doi:10.1258/ebm.2011.011107 (2011).
22. Rang, F. J., Kloosterman, W. P. & de Ridder, J. From squiggle to basepair: computational approaches for improving nanopore sequencing read accuracy. Genome Biol 19, 90, doi:10.1186/s13059-018-1462-9 (2018).
23. Joffroy, B., Uca, Y. O., Presern, D., Doye, J. P. K. & Schmidt, T. L. Rolling circle amplification shows a sinusoidal template length-dependent amplification bias. Nucleic Acids Res 46, 538-545, doi:10.1093/nar/gkx1238 (2018).
24. Soh, Y. Q. et al. Sequencing the mouse Y chromosome reveals convergent gene acquisition and amplification on both sex chromosomes. Cell 159, 800-813, doi:10.1016/j.cell.2014.09.052 (2014).
25. Sakahira, H., Enari, M. & Nagata, S. Cleavage of CAD inhibitor in CAD activation and DNA degradation during apoptosis. Nature 391, 96-99, doi:10.1038/34214 (1998).
26. Li, L. Y., Luo, X. & Wang, X. Endonuclease G is an apoptotic DNase when released from mitochondria. Nature 412, 95-99, doi:10.1038/35083620 (2001).
27. Mizuta, R. et al. DNase gamma is the effector endonuclease for internucleosomal DNA fragmentation in necrosis. PLoS One 8, e80223, doi:10.1371/journal.pone.0080223 (2013).
28. Arakawa, H. et al. Functional redundancy between DNA ligases I and III in DNA replication in vertebrate cells. Nucleic Acids Res 40, 2599-2610, doi:10.1093/nar/gkr1024 (2012).
29. Lu, G. et al. Ligase I and ligase III mediate the DNA double-strand break ligation in alternative end-joining. Proc Natl Acad Sci USA 113, 1256-1260, doi:10.1073/pnas.1521597113 (2016).
30. Simsek, D. et al. Crucial role for DNA ligase III in mitochondria but not in Xrcc1-dependent repair. Nature 471, 245-248, doi:10.1038/nature09794 (2011).
31. Marichal, T. et al. DNA released from dying host cells mediates aluminum adjuvant activity. Nat Med 17, 996-1002, doi:10.1038/nm.2403 (2011).
32. Tada, R. et al. Essential Role of Host Double-Stranded DNA Released from Dying Cells by Cationic Liposomes for Mucosal Adjuvanticity. Vaccines (Basel) 8, doi:10.3390/vaccines8010008 (2019).
33. Li, Y., Berke, I. C. & Modis, Y. DNA binding to proteolytically activated TLR9 is sequence-independent and enhanced by DNA curvature. EMBO J 31, 919-931, doi:10.1038/emboj.2011.441 (2012).
34. Lange, S. S. & Vasquez, K. M. HMGB1: the jack-of-all-trades protein is a master DNA repair mechanic. Mol Carcinog 48, 571-580, doi:10.1002/mc.20544 (2009).
35. Jin, D. & Sprent, J. GM-CSF Culture Revisited: Preparation of Bulk Populations of Highly Pure Dendritic Cells from Mouse Bone Marrow. J Immunol 201, 3129-3139, doi:10.4049/jimmunol.1800031 (2018).
36. Iwasaki, A. & Medzhitov, R. Control of adaptive immunity by the innate immune system. Nat Immunol 16, 343-353, doi:10.1038/ni.3123 (2015).
37. Wang, Z. et al. Regulation of innate immune responses by DAI (DLM-1/ZBP1) and other DNA-sensing molecules. Proc Natl Acad Sci USA 105, 5477-5482, doi:10.1073/pnas.0801295105 (2008).
38. Green, D. R., Ferguson, T., Zitvogel, L. & Kroemer, G. Immunogenic and tolerogenic cell death. Nat Rev Immunol 9, 353-363, doi:10.1038/nri2545 (2009).
39. Assouvie, A., Daley-Bauer, L. P. & Rousselet, G. Growing Murine Bone Marrow-Derived Macrophages. Methods Mol Biol 1784, 29-33, doi:10.1007/978-1-4939-7837-3_3 (2018).
40. Sundaresan, R., Parameshwaran, H. P., Yogesha, S. D., Keilbarth, M. W. & Rajan, R. RNA-Independent DNA Cleavage Activities of Cas9 and Cas12a. Cell Rep 21, 3728-3739, doi:10.1016/j.celrep.2017.11.100 (2017).
41. Putney, S. D., Benkovic, S. J. & Schimmel, P. R. A DNA fragment with an alpha-phosphorothioate nucleotide at one end is asymmetrically blocked from digestion by exonuclease III and can be replicated in vivo. Proc Natl Acad Sci USA 78, 7350-7354, doi:10.1073/pnas.78.12.7350 (1981).
42. Boada-Romero, E., Martinez, J., Heckmann, B. L. & Green, D. R. The clearance of dead cells by efferocytosis. Nat Rev Mol Cell Biol 21, 398-414, doi:10.1038/s41580-020-0232-1 (2020).
43. McWhirter, S. M. & Jefferies, C. A. Nucleic Acid Sensors as Therapeutic Targets for Human Disease. Immunity 53, 78-97, doi:10.1016/j.immuni.2020.04.004 (2020).
44. Sauer, J. D. et al. The N-ethyl-N-nitrosourea-induced Goldenticket mouse mutant reveals an essential function of Sting in the in vivo interferon response to Listeria monocytogenes and cyclic dinucleotides. Infect Immun 79, 688-694, doi:10.1128/IAI.00999-10 (2011).
45. Hou, B., Reizis, B. & DeFranco, A. L. Toll-like receptors activate innate and adaptive immunity by using dendritic cell-intrinsic and -extrinsic mechanisms. Immunity 29, 272-282, doi:10.1016/j.immuni.2008.05.016 (2008).
46. Kijima, M. & Mizuta, R. Histone H1 quantity determines the efficiencies of apoptotic DNA fragmentation and chromatin condensation. Biomed Res 40, 51-56, doi:10.2220/biomedres.40.51 (2019).
47. Shiokawa, D., Kobayashi, T. & Tanuma, S. Involvement of DNase gamma in apoptosis associated with myogenic differentiation of C2C12 cells. J Biol Chem 277, 31031-31037, doi:10.1074/jbc.M204038200 (2002).
48. Shiokawa, D. & Tanuma, S. Differential DNases are selectively used in neuronal apoptosis depending on the differentiation state. Cell Death Differ 11, 1112-1120, doi:10.1038/sj.cdd.4401454 (2004).
49. Collins, J. A., Schandi, C. A., Young, K. K., Vesely, J. & Willingham, M. C. Major DNA fragmentation is a late event in apoptosis. J Histochem Cytochem 45, 923-934, doi:10.1177/002215549704500702 (1997).
50. Wang, H. et al. DNA ligase III as a candidate component of backup pathways of nonhomologous end joining. Cancer Res 65, 4020-4030, doi:10.1158/0008-5472.CAN-04-3055 (2005).
51. Soni, A. et al. Requirement for Parp-1 and DNA ligases 1 or 3 but not of Xrcc1 in chromosomal translocation formation by backup end joining. Nucleic Acids Res 42, 6380-6392, doi:10.1093/nar/gku298 (2014).
52. Simsek, D. et al. DNA ligase III promotes alternative nonhomologous end-joining during chromosomal translocation formation. PLoS Genet 7, e1002080, doi:10.1371/journal.pgen.1002080 (2011).
53. Shoshani, O. et al. Chromothripsis drives the evolution of gene amplification in cancer.

Nature, doi:10.1038/s41586-020-03064-z (2020).
54. Moller, H. D. et al. CRISPR-C: circularization of genes and chromosome by CRISPR in human cells. Nucleic Acids Res 46, e131, doi:10.1093/nar/gky767 (2018).
55. Zhang, Y. et al. The fundamental role of chromatin loop extrusion in physiological V(D)J recombination. Nature 573, 600-604, doi:10.1038/s41586-019-1547-y (2019).
56. Riley, J. S. & Tait, S. W. Mitochondrial DNA in inflammation and immunity. EMBO Rep 21, e49799, doi:10.15252/embr.201949799 (2020).
57. Li, X. D. et al. Pivotal roles of cGAS-cGAMP signaling in antiviral defense and immune adjuvant effects. Science 341, 1390-1394, doi:10.1126/science.1244040 (2013).
58. Andreeva, L. et al. cGAS senses long and HMGB/TFAM-bound U-turn DNA by forming protein-DNA ladders. Nature 549, 394-398, doi:10.1038/nature23890 (2017).
59. Tovey, M. G., Lallemand, C. & Thyphronitis, G. Adjuvant activity of type I interferons.

Biol Chem 389, 541-545, doi:10.1515/bc.2008.051 (2008).
60. Moore, J. B. & June, C. H. Cytokine release syndrome in severe COVID-19. Science 368, 473-474, doi:10.1126/science.abb8925 (2020).
61. Li, H. Minimap2: pairwise alignment for nucleotide sequences. Bioinformatics 34, 3094-3100, doi:10.1093/bioinformatics/bty191 (2018).
62. Quinlan, A. R. & Hall, I. M. BEDTools: a flexible suite of utilities for comparing genomic features. Bioinformatics 26, 841-842, doi:10.1093/bioinformatics/btq033 (2010).
63. Gel, B. & Serra, E. karyoploteR: an R/Bioconductor package to plot customizable genomes displaying arbitrary data. Bioinformatics 33, 3088-3090, doi:10.1093/bioinformatics/btx346 (2017).
64. Bolger, A. M., Lohse, M. & Usadel, B. Trimmomatic: a flexible trimmer for Illumina sequence data. Bioinformatics 30, 2114-2120, doi:10.1093/bioinformatics/btu170 (2014).
65. Li, H. & Durbin, R. Fast and accurate short read alignment with Burrows-Wheeler transform. Bioinformatics 25, 1754-1760, doi:10.1093/bioinformatics/btp324 (2009).
66. Ramirez, F. et al. deepTools2: a next generation web server for deep-sequencing data analysis. Nucleic acids research 44, W160-165, doi:10.1093/nar/gkw257 (2016).
67. Frankish, A. et al. GENCODE reference annotation for the human and mouse genomes. Nucleic acids research 47, D766-D773, doi:10.1093/nar/gky955 (2019).
68. Dobin, A. et al. STAR: ultrafast universal RNA-seq aligner. Bioinformatics 29, 15-21, doi:10.1093/bioinformatics/bts635 (2013).
69. Li, B. & Dewey, C. N. RSEM: accurate transcript quantification from RNA-Seq data with or without a reference genome. BMC bioinformatics 12, 323, doi:10.1186/1471-2105-12-323 (2011).
70. Love, M. I., Huber, W. & Anders, S. Moderated estimation of fold change and dispersion for RNA-seq data with DESeq2. Genome biology 15, 550, doi:10.1186/s13059-014-0550-8 (2014).
71. Hirt, B. Selective extraction of polyoma DNA from infected mouse cell cultures. J Mol Biol 26, 365-369, doi:10.1016/0022-2836(67)90307-5 (1967).
72. Goebel, W. & Schrempf, H. Isolation and characterization of supercoiled circular deoxyribonucleic acid from beta-hemolytic strains of Escherichia coli. J Bacteriol 106, 311-317, doi:10.1128/jb.106.2.311-317.1971 (1971).
73. Mukai, T., Matsubara, K. & Takagi, Y. Isolation of circular DNA molecules from whole cellular DNA by use of ATP-dependent deoxyribonuclease. Proc Natl Acad Sci USA 70, 2884-2887, doi:10.1073/pnas.70.10.2884 (1973).
74. Yamagishi, H. et al. Purification of small polydisperse circular DNA of eukaryotic cells by use of ATP-dependent deoxyribonuclease. Gene 26, 317-321, doi:10.1016/0378-1119(83)90205-6 (1983).
75. Radloff, R., Bauer, W. & Vinograd, J. A dye-buoyant-density method for the detection and isolation of closed circular duplex DNA: the closed circular DNA in HeLa cells.

Proc Natl Acad Sci USA 57, 1514-1521, doi:10.1073/pnas.57.5.1514 (1967).
76. Hardman, N. Formation of rings from segments of HeLa-cell nuclear deoxyribonucleic acid. Biochem J 143, 521-534, doi:10.1042/bj1430521 (1974).
77. Machwe, A., Ganunis, R., Bohr, V. A. & Orren, D. K. Selective blockage of the 3′→5′ exonuclease activity of WRN protein by certain oxidative modifications and bulky lesions in DNA. Nucleic Acids Res 28, 2762-2770, doi:10.1093/nar/28.14.2762 (2000).
78. Pizzolato, J., Mukherjee, S., Scharer, O. D. & Jiricny, J. FANCD2-associated nuclease 1, but not exonuclease 1 or flap endonuclease 1, is able to unhook DNA interstrand cross-links in vitro. J Biol Chem 290, 22602-22611, doi:10.1074/jbc.M115.663666 (2015).
79. Cote, A. G. In vivo analysis of cruciform extrusion and resolution of DNA palindromes in eukaryotes. Doctoral thesis, University of Toronto, Ontario, CA (2009).
80. Ali, N., Rampazzo, R. C. P., Costa, A. D. T. & Krieger, M. A. Current Nucleic Acid Extraction Methods and Their Implications to Point-of-Care Diagnostics. Biomed Res Int 2017, 9306564, doi:10.1155/2017/9306564 (2017).
81. Moller, H. D., Parsons, L., Jorgensen, T. S., Botstein, D. & Regenberg, B. Extrachromosomal circular DNA is common in yeast. Proc Natl Acad Sci USA 112, E3114-3122, doi:10.1073/pnas.1508825112 (2015).
82. Hotchkiss, R. S. et al. Apoptotic cell death in patients with sepsis, shock, and multiple organ dysfunction. Crit Care Med 27, 1230-1251, doi:10.1097/00003246-199907000-00002 (1999).
83. Leisman, D. E. et al. Cytokine elevation in severe and critical COVID-19: a rapid systematic review, meta-analysis, and comparison with other inflammatory syndromes. Lancet Respir Med 8, 1233-1244, doi:10.1016/S2213-2600(20)30404-5 (2020).

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method for enriching extrachromosomal circular DNA (eccDNA) from a sample, comprising:
(a) obtaining a sample comprising unenriched eccDNA;
(b) treating the sample comprising unenriched eccDNA obtained in (a) with an enzyme that linearizes mitochondrial DNA (mtDNA), under conditions under which mtDNA is linearized, thereby producing a mixture of linearized DNA and eccDNA;
(c) treating the mixture produced in (b) with an enzyme that digests linear DNA, thereby producing a mixture of digested linear DNA and eccDNA; and
(d) separating eccDNA from the digested linear DNA in the mixture produced in (c), thereby producing enriched eccDNA.

2. The method of claim 1, wherein in (a) the sample is a biological sample collected from an individual.

3. The method of claim 2, wherein the biological sample is a blood or plasma sample.

4. The method of claim 2 or 3, wherein the individual is a mammal.

5. The method of claim 4, wherein the mammal is a human.

6. The method of claim 1, wherein in (a) the sample comprises a population of cells.

7. The method of claim 6, wherein in (a) the sample comprises a population of cultured cells.

8. The method of claim 6, wherein the population of cultured cells is a population of mammalian cells.

9. The method of any one of claims 6-8, wherein prior to (b) the population of cells is fixed.

10. The method of any one of claims 6-9, wherein prior to (b) the population of cells is lysed.

11. The method of claim 10, wherein the population of cells is lysed by buffered alkaline lysis conducted at a pH between 11.0 and 12.3.

12. The method of claim 11, wherein buffered alkaline lysis is conducted at a pH of about 11.8.

13. The method of claim 11 or 12, wherein buffered alkaline lysis is conducted in the presence of a compound that has an acid dissociation pH (pKa) between 11.0 and 12.3.

14. The method of claim 13, wherein the compound is pyrrolidine.

15. The method of any one of claims 10-14, wherein prior to (b) the unenriched eccDNA is bound to magnetic silica beads to separate eccDNA from lysed cells in the sample.

16. The method of any one of claims 1-15, wherein in (b) the enzyme that linearizes mtDNA is PacI.

17. The method of any one of claims 1-16, wherein in (c) the enzyme that digests linear DNA is a plasmid safe DNase or Exonuclease V.

18. The method of any one of claims 1-17, wherein (b) and (c) are conducted concurrently.

19. The method of any one of claims 1-18, wherein in (d) the eccDNA is separated from the digested linear mtDNA by phenol/chloroform/isoamyl alcohol (PCI) extraction.

20. The method of any one of claims 1-19, wherein in (d) the eccDNA is separated from the digested linear mtDNA by contacting the product of (c) with magnetic silica beads.

21. The method of any one of claims 1-20, further comprising amplifying the enriched eccDNA by rolling circle amplification.

22. A method for enriching extrachromosomal circular DNA (eccDNA) from a mixture comprising circular DNA and linear DNA, comprising:
(a) obtaining the mixture comprising circular DNA and linear DNA;
(b) treating the mixture obtained in (a) with an enzyme that linearizes circular DNA that is not eccDNA, under conditions under which such DNA is linearized, thereby producing a mixture of linearized DNA and eccDNA;
(c) treating the mixture produced in (b) with an enzyme that digests linear DNA, thereby producing a mixture of digested linear DNA and eccDNA; and
(d) separating eccDNA from the digested linear DNA in the mixture produced in (c), thereby producing enriched eccDNA.

23. A composition comprising immunostimulatory extrachromosomal circular DNA (eccDNA) and a pharmaceutically acceptable excipient.

24. The composition of claim 23, wherein the eccDNA is produced by any one of the methods of claims A1-A13.

25. The composition of claim 23 or 24, wherein the eccDNA is derived from chromosomal DNA of a mammal.

26. The composition of claim 25, wherein the mammal is a human.

27. The composition of any one of claims 23-26, wherein the eccDNA is essentially free of bacterial, bacteriophage, and plasmid sequences.

28. The composition of any one of claims 23-27, wherein the eccDNA is essentially free of sequences encoding site-specific recombination sites.

29. The composition of any one of claims 23-28, wherein the eccDNA is non-replicating.

30. The composition of any one of claims 23-29, wherein the eccDNA has a size range of about 70 nucleotides to about 2000 nucleotides.

31. The composition of any one of claims 23-30, further comprising an antigen.

32. The composition of claim 31, wherein the antigen is a peptide, protein, or nucleic acid.

33. The composition of claim 31 or 32, wherein the antigen is an antigen from a pathogen.

34. The composition of claim 33, wherein the pathogen is a bacterium, a virus, or a parasite.

35. A composition comprising immunostimulatory circular DNA and a pharmaceutically acceptable excipient, wherein the circular DNA is non-replicating and does not comprise a gene that is capable of being expressed.

36. The composition of claim 35, wherein the circular DNA comprises a random DNA sequence.

37. The composition of claim 36, wherein the circular DNA comprises an entirely random DNA sequence.

38. The composition of any one of claims 35-37, wherein the circular DNA has a size range of about 70 nucleotides to about 2000 nucleotides.

39. The composition of any one of claims 35-38, wherein the circular DNA is synthesized in vitro.

40. The composition of any one of claims 35-39, further comprising an antigen.

41. The composition of claim 40, wherein the antigen is a peptide, protein, or nucleic acid.

42. The composition of claim 40 or 41, wherein the antigen is an antigen from a pathogen.

43. The composition of claim 42, wherein the pathogen is a bacterium, a virus, or a parasite.

44. A method for eliciting an immune response in an individual, the method comprising administering to the individual an effective amount of a composition comprising immunostimulatory circular DNA and a pharmaceutically acceptable excipient, wherein the circular DNA is non-replicating and does not comprise a gene that is capable of being expressed.

45. The method of claim 44, wherein the circular DNA has been obtained from the individual.

46. The method of claim 44 or 45, wherein the circular DNA has been obtained from a second individual.

47. The method of claims 44 or 45, wherein the circular DNA has been obtained from a population of cultured cells.

48. The method of any one of claims 44-47, wherein the circular DNA comprises eccDNA derived from chromosomal DNA of a mammal.

49. The method of claim 48, wherein the circular DNA comprises eccDNA derived from chromosomal DNA of a human.

50. The method of claim 44, wherein the circular DNA has been synthesized in vitro.

51. The method of claim 50, wherein the circular DNA comprises a random sequence.

52. The method of claim 51, wherein the circular DNA comprises an entirely random sequence.

53. The method of any one of claims 44-52, wherein the circular DNA has been amplified by rolling circle amplification.

54. The method of any one of claims 44-53, wherein the circular DNA is essentially free of bacterial, bacteriophage, and plasmid sequences.

55. The method of any one of claims 44-54, wherein the circular DNA is essentially free of sequences encoding site-specific recombination sites.

56. The method of any one of claims 44-55, wherein the circular DNA has a size range of about 70 nucleotides to about 2000 nucleotides.

57. The method of any one of claims 44-56, wherein the composition administered to the individual further comprises an antigen.

58. The method of claim 57, wherein the antigen is a peptide, protein, or nucleic acid.

59. The method of claim 57 or 58, wherein the antigen is an antigen from a pathogen.

60. The method of claim 59, wherein the pathogen is a bacterium, virus, or parasite.

61. The method of any one of claims 44-60, wherein administration of the composition to the individual activates cGAS-STING signaling in cells of the individual.

62. The method of any one of claims 44-61, wherein administration of the composition to the individual elicits an innate immune response in the individual.

63. The method of any one of claims 44-62, wherein administration of the composition to the individual elicits a cell-mediated immune response in the individual.

64. The method of claim 63, wherein administration of the composition to the individual elicits proliferation of mature T helper type 1 (Th1) cells.

65. The method of any one of claims 44-64, wherein administration of the composition to the individual elicits an increase in inflammatory cytokines.

66. The method of claim 65, wherein the one or more of the inflammatory cytokines are selected from: interferon alpha (IFNα), interferon beta (IFNβ), interferon gamma (IFNγ), interleukin 6 (IL-6), and tumor necrosis factor alpha (TNFα).

67. The method of any one of claims 44-66, wherein the immune response is elicited in the individual to treat a disease or condition.

68. The method any one of claims 44-66, wherein the immune response is elicited in the individual to prevent a disease or condition or reduce the extent to which the disease or condition occurs.

69. The method of claim 67 or 68, wherein the disease or condition is cancer.

70. The method of claim 67 or 68, wherein the disease or condition is caused by a pathogen.

71. The method of claim 70, wherein the disease or condition is caused by a bacterium, a virus, or a parasite.

72. The method of any one of claims 44-71, wherein the administration is intravenous, intramuscular, intradermal, intranasal, topical, or oral.

73. The method of any one of claims 44-72, wherein the individual is a mammal.

74. The method of claim 73, wherein the mammal is a human.

75. An immunostimulatory composition comprising circular DNA and a pharmaceutically acceptable excipient,
wherein the immunostimulatory composition elicits an immune response in an individual when administered to the individual, and
wherein the circular DNA is non-replicating and does not comprise a gene that is capable of being expressed.

76. The immunostimulatory composition of claim 75, wherein the circular DNA comprises eccDNA derived from chromosomal DNA of a mammal.

77. The immunostimulatory composition of claim 76, wherein the circular DNA comprises eccDNA derived from chromosomal DNA of a human.

78. The immunostimulatory composition of claim 75, wherein the circular DNA is synthesized in vitro.

79. The immunostimulatory composition of claim 78, wherein the circular DNA comprises a random sequence.

80. The immunostimulatory composition of claim 79, wherein the circular DNA comprises an entirely random sequence.

81. The immunostimulatory composition of any one of claims 75-80, wherein the circular DNA is essentially free of bacterial, bacteriophage, and plasmid sequences.

82. The immunostimulatory composition of any one of claims 75-81, wherein the circular DNA is essentially free of sequences encoding site-specific recombination sites.

83. The immunostimulatory composition of any one of claims 75-82, wherein the circular DNA has a size range of about 70 nucleotides to about 2000 nucleotides.

84. The immunostimulatory composition of any one of claims 75-83, wherein the composition further comprises an antigen.

85. The immunostimulatory composition of claim 84, wherein the antigen is a peptide, protein, or nucleic acid.

86. The immunostimulatory composition of claim 84 or 85, wherein the antigen is an antigen from a pathogen.

87. The immunostimulatory composition of claim 86, wherein the antigen is a bacterial, viral, or parasite antigen.

88. The immunostimulatory composition of any one of claims 75-87, wherein the circular DNA enhances the immunogenicity of the antigen when co-administered to an individual.

89. The immunostimulatory composition of any one of claims 75-88, wherein administration of the composition to the individual activates cGAS-STING signaling in cells of the individual.

90. The immunostimulatory composition of any one of claims 75-89, wherein administration of the composition to the individual elicits an innate immune response in the individual.

91. The immunostimulatory composition of any one of claims 75-90, wherein administration of the composition to the individual elicits a cell-mediated immune response in the individual.

92. The immunostimulatory composition of claim 91, wherein administration of the composition to the individual elicits proliferation of mature T helper type 1 (Th1) cells in the individual.

93. The immunostimulatory composition of any one of claims 75-92, wherein administration of the composition to the individual elicits an increase in inflammatory cytokines in the individual.

94. The immunostimulatory composition of any one of claims 75-93, wherein the individual is a mammal.

95. The immunostimulatory composition of claim 94, wherein the mammal is a human.

96. A method for assessing a disease in an individual known, suspected to have, or at risk for a disease associated with an increase in the level of extrachromosomal circular DNA (eccDNA), comprising:
(a) obtaining a sample comprising eccDNA from the individual;
(b) measuring the level of eccDNA in the sample; and
(c) comparing the level of eccDNA measured in the sample to a reference level of eccDNA for the disease,
wherein a statistical similarity between the level of eccDNA measured in the sample and the reference level is indicative of the disease.

97. The method of claim 96, wherein the sample is a blood or plasma sample.

98. The method of claim 96 or 97, wherein prior to (b) the eccDNA is enriched by the method of any one of claims 1-21.

99. The method of any one of claims 96-98, wherein the individual is a human.

100. The method of any one of claims 96-99, wherein the disease is caused by a pathogen.

101. The method of claim 100, wherein the disease is sepsis, acute respiratory distress syndrome (ARDS), CAR T cell-induced cytokine release syndrome (CRS), or coronavirus disease 2019 (COVID-19).


SEQUENCE LISTING (57 sequences)

SEQ ID NO: 1 - DNA, 24 bp, synthetic construct
  source 1..24: mol_type = other DNA
caccgcagaacattcgtttgagga

SEQ ID NO: 2 - DNA, 24 bp, synthetic construct
  source 1..24: mol_type = other DNA
aaactcctcaaacgaatgttctgc

SEQ ID NO: 3 - DNA, 25 bp, synthetic construct
  source 1..25: mol_type = other DNA
caccgcatggtctcaccagtaaagg

SEQ ID NO: 4 - DNA, 25 bp, synthetic construct
  source 1..25: mol_type = other DNA
aaaccctttactggtgagaccatgc

SEQ ID NO: 5 - DNA, 25 bp, synthetic construct
  source 1..25: mol_type = other DNA
caccgttggggattgaaacgcgtga

SEQ ID NO: 6 - DNA, 25 bp, synthetic construct
  source 1..25: mol_type = other DNA
aaactcacgcgtttcaatccccaac

SEQ ID NO: 7 - DNA, 25 bp, synthetic construct
  source 1..25: mol_type = other DNA
caccgccgagtgccattgttgccgg

SEQ ID NO: 8 - DNA, 25 bp, synthetic construct
  source 1..25: mol_type = other DNA
aaacccggcaacaatggcactcggc

SEQ ID NO: 9 - DNA, 25 bp, synthetic construct
  source 1..25: mol_type = other DNA
caccgtggtaaggcaggtcagacgt

SEQ ID NO: 10 - DNA, 25 bp, synthetic construct
  source 1..25: mol_type = other DNA
aaacacgtctgacctgccttaccac

SEQ ID NO: 11 - DNA, 20 bp, synthetic construct
  source 1..20: mol_type = other DNA
gtgggtgtggaggctctttt

SEQ ID NO: 12 - DNA, 20 bp, synthetic construct
  source 1..20: mol_type = other DNA
gattctggcctgtaggcact

SEQ ID NO: 13 - DNA, 21 bp, synthetic construct
  source 1..21: mol_type = other DNA
aagggagctgactccttctca

SEQ ID NO: 14 - DNA, 20 bp, synthetic construct
  source 1..20: mol_type = other DNA
accccacaccttttgttgct

SEQ ID NO: 15 - DNA, 20 bp, synthetic construct
  source 1..20: mol_type = other DNA
cacacctcggaccttccatt

SEQ ID NO: 16 - DNA, 20 bp, synthetic construct
  source 1..20: mol_type = other DNA
gtggaacgaccaatggcaag

SEQ ID NO: 17 - DNA, 20 bp, synthetic construct
  source 1..20: mol_type = other DNA
taacaagcacgagtgggagg

SEQ ID NO: 18 - DNA, 25 bp, synthetic construct
  source 1..25: mol_type = other DNA
caccgcttctgctgacataagggag

SEQ ID NO: 19 - DNA, 25 bp, synthetic construct
  source 1..25: mol_type = other DNA
aaacctcccttatgtcagcagaagc

SEQ ID NO: 20 - DNA, 24 bp, synthetic construct
  source 1..24: mol_type = other DNA
caccggaccaccctgattctgtgg

SEQ ID NO: 21 - DNA, 24 bp, synthetic construct
  source 1..24: mol_type = other DNA
aaacccacagaatcagggtggtcc

SEQ ID NO: 22 - DNA, 25 bp, synthetic construct
  source 1..25: mol_type = other DNA
caccgccgctgctctgccatcgcac

SEQ ID NO: 23 - DNA, 25 bp, synthetic construct
  source 1..25: mol_type = other DNA
aaacgtgcgatggcagagcagcggc

SEQ ID NO: 24 - DNA, 25 bp, synthetic construct
  source 1..25: mol_type = other DNA
caccgtgagaaactggaacgggctc

SEQ ID NO: 25 - DNA, 25 bp, synthetic construct
  source 1..25: mol_type = other DNA
aaacgagcccgttccagtttctcac

SEQ ID NO: 26 - DNA, 24 bp, synthetic construct
  source 1..24: mol_type = other DNA
caccgtggaacataagtctgcaaa

SEQ ID NO: 27 - DNA, 24 bp, synthetic construct
  source 1..24: mol_type = other DNA
aaactttgcagacttatgttccac

SEQ ID NO: 28 - DNA, 24 bp, synthetic construct
  source 1..24: mol_type = other DNA
caccggtgtccgattcagtagaca

SEQ ID NO: 29 - DNA, 24 bp, synthetic construct
  source 1..24: mol_type = other DNA
aaactgtctactgaatcggacacc

SEQ ID NO: 30 - DNA, 24 bp, synthetic construct
  source 1..24: mol_type = other DNA
caccgctttatcagttcaaaccgg

SEQ ID NO: 31 - DNA, 24 bp, synthetic construct
  source 1..24: mol_type = other DNA
aaacccggtttgaactgataaagc

SEQ ID NO: 32 - DNA, 24 bp, synthetic construct
  source 1..24: mol_type = other DNA
caccgtatttgctttagagcttgc

SEQ ID NO: 33 - DNA, 24 bp, synthetic construct
  source 1..24: mol_type = other DNA
aaacgcaagctctaaagcaaatac

SEQ ID NO: 34 - DNA, 200 bp, synthetic construct
  source 1..200: mol_type = other DNA
catcctctcacagcaccatgcaggccggcgtacgggtcccatataaacctgtcatagcttacctgactctacttggaaatgtggctaggtctttgcccacgcacctaatcggtcctcgtttgctaggcctgacccgatgaactacagaacactgcaagaatctctacctgctttacaaagtgctggatcctattccagcg

SEQ ID NO: 35 - DNA, 200 bp, synthetic construct
  source 1..200: mol_type = other DNA
cgcacctaatcggtcctcgtttgctaggcctgacccgatgaactacagaacactgcaagaatctctacctgctttacaaagtgctggatcctattccagcgcatcctctcacagcaccatgcaggccggcgtacgggtcccatataaacctgtcatagcttacctgactctacttggaaatgtggctaggtctttgccca

SEQ ID NO: 36 - DNA, 21 bp, synthetic construct
  source 1..21: mol_type = other DNA
catcctctcacagcaccatgc

SEQ ID NO: 37 - DNA, 21 bp, synthetic construct
  source 1..21: mol_type = other DNA
cgctggaataggatccagcac

SEQ ID NO: 38 - DNA, 22 bp, synthetic construct
  source 1..22: mol_type = other DNA
tgggcaaagacctagccacatt

SEQ ID NO: 39 - DNA, 19 bp, synthetic construct
  source 1..19: mol_type = other DNA
cgcacctaatcggtcctcg

SEQ ID NO: 40 - DNA, 525 bp, synthetic construct
  source 1..525: mol_type = other DNA
gactcacaggaaaggtgcgtatagagcccagcgcaaagagggagagctcaaatgatgttacttagagaacctcagtaatgatttcacgcggggaaagctcaaacaagtgctatacatggattactcggtgacttcgatggaaatcacccgtgtgtgggtaccgcgtataatcgcgttccttgtaatgaagtatgttgccgtttcatgtgtacagcattatggtcaacgaagcattgctctcttgaacagaagctattggtccacttcatgtgcataaagataagtatcatacccgcactttacataggatagtttaatcttgctttgcaatagaacggttcttcggcttgggagggtacaaagccgatgagactcacacttccccgtacttaccttcgttcgttggctcgacttaccgataaatacgctagagacgtctagacccagtggtgtttacctataagaccatattcagtgcagctttcatagaatcggcctcatatgtgcttaggtctacccgatttg

SEQ ID NO: 41 - DNA, 525 bp, synthetic construct
  source 1..525: mol_type = other DNA
tattggtccacttcatgtgcataaagataagtatcatacccgcactttacataggatagtttaatcttgctttgcaatagaacggttcttcggcttgggagggtacaaagccgatgagactcacacttccccgtacttaccttcgttcgttggctcgacttaccgataaatacgctagagacgtctagacccagtggtgtttacctataagaccatattcagtgcagctttcatagaatcggcctcatatgtgcttaggtctacccgatttggactcacaggaaaggtgcgtatagagcccagcgcaaagagggagagctcaaatgatgttacttagagaacctcagtaatgatttcacgcggggaaagctcaaacaagtgctatacatggattactcggtgacttcgatggaaatcacccgtgtgtgggtaccgcgtataatcgcgttccttgtaatgaagtatgttgccgtttcatgtgtacagcattatggtcaacgaagcattgctctcttgaacagaagc

SEQ ID NO: 42 - DNA, 20 bp, synthetic construct
  source 1..20: mol_type = other DNA
gactcacaggaaaggtgcgt

SEQ ID NO: 43 - DNA, 23 bp, synthetic construct
  source 1..23: mol_type = other DNA
caaatcgggtagacctaagcaca

SEQ ID NO: 44 - DNA, 20 bp, synthetic construct
  source 1..20: mol_type = other DNA
tattggtccacttcatgtgc

SEQ ID NO: 45 - DNA, 20 bp, synthetic construct
  source 1..20: mol_type = other DNA
gcttctgttcaagagagcaa

SEQ ID NO: 46 - DNA, 21 bp, synthetic construct
  source 1..21: mol_type = other DNA
aggtcggtgtgaacggatttg

SEQ ID NO: 47 - DNA, 23 bp, synthetic construct
  source 1..23: mol_type = other DNA
tgtagaccatgtagttgaggtca

SEQ ID NO: 48 - DNA, 19 bp, synthetic construct
  source 1..19: mol_type = other DNA
tgatggtcttggtggtgat

SEQ ID NO: 49 - DNA, 18 bp, synthetic construct
  source 1..18: mol_type = other DNA
ttgtgccaggagtgtcaa

SEQ ID NO: 50 - DNA, 21 bp, synthetic construct
  source 1..21: mol_type = other DNA
ggtggaatgagactattgttg

SEQ ID NO: 51 - DNA, 19 bp, synthetic construct
  source 1..19: mol_type = other DNA
cttcaagtggagagcagtt

SEQ ID NO: 52 - DNA, 21 bp, synthetic construct
  source 1..21: mol_type = other DNA
tgccaccttttgacagtgatg

SEQ ID NO: 53 - DNA, 20 bp, synthetic construct
  source 1..20: mol_type = other DNA
tgtgctgctgcgagatttga

SEQ ID NO: 54 - DNA, 20 bp, synthetic construct
  source 1..20: mol_type = other DNA
cggccttccctacttcacaa

SEQ ID NO: 55 - DNA, 22 bp, synthetic construct
  source 1..22: mol_type = other DNA
ttgccattgcacaactcttttc

SEQ ID NO: 56 - DNA, 21 bp, synthetic construct
  source 1..21: mol_type = other DNA
gtccccaaagggatgagaagt

SEQ ID NO: 57 - DNA, 20 bp, synthetic construct
  source 1..20: mol_type = other DNA
tttgctacgacgtgggctac